(19) United States
(12) Patent Application Publication
(10) Pub. No.: US 2023/0093782 A9
(43) Pub. Date: Mar. 23, 2023

(54) SUBUNIT VACCINE CONSTRUCTS FOR FLAVIVIRUSES

(71) Applicants: REGENTS OF THE UNIVERSITY OF MINNESOTA, Minneapolis, MN (US); KANPRO RESEARCH, INC., Lawrence, KS (US)

(72) Inventors: Sunil A. David, Minneapolis, MN (US); Michael J.H. Brush, Minneapolis, MN (US); Fei Philip Gao, Lawrence, KS (US)

(21) Appl. No.: 17/312,534

(22) PCT Filed: Dec. 12, 2019
(86) PCT No.: PCT/US2019/065886

§ 371 Date: Jun. 10, 2021

Related U.S. Application Data
(60) Provisional application No. 62/778,347, filed on Dec. 12, 2018.

(51) Int. Cl. A61K 39/12; C07K 14/005; C12N 7/00

(52) U.S. Cl. CPC A61K 39/12; C07K 14/005; C12N 7/00; A61K 2039/55511

(57) ABSTRACT

This disclosure describes a subunit vaccine for a flavivirus, methods of making the vaccine, and methods of using the vaccine. The flavivirus may include, is a mosquito-borne flavivims, for example, Zika virus (ZIKV), dengue virus (DENV), Yellow Fever (YF) virus, and West Nile Virus (WNV). The subunit vaccine may be administered with an adjuvant.

### RELATED APPLICATION

This application claims the benefit of U.S. Provisional Application No. 62/778,347, filed Dec. 12, 2018, the disclosure of which is incorporated by reference herein in its entirety.

### GOVERNMENT FUNDING

This invention was made with government support under 272201400056C awarded by the National Institutes of Health. The government has certain rights in the invention.

### SEQUENCE LISTING

This application contains a Sequence Listing electronically submitted via EFS-Web to the United States Patent and Trademark Office as an ASCII text file entitled “0110_000581WO01_SL.txt” having a size of 74,966 bytes and created on Dec. 4, 2019. The information contained in the Sequence Listing is incorporated by reference herein.

### BACKGROUND

The mosquito-borne flavivirus Zika virus (ZIKV), first discovered in Uganda in 1947,¹⁷ was consigned to obscurity for nearly 70 years, with sporadic cases occurring in Africa and Asia. In March 2015, however, ZIKV was identified in association with an outbreak of exanthematous illness in Brazil;^{19, 20} and by December 2015, the virus had spread widely in Brazil, resulting in an estimated 1.3 million cases with a presumptive diagnosis of ZIKV infection.²¹ ZIKV is now known to be a cause of congenital neurologic birth defects, notably microcephaly²²⁻²⁸ and has also been associated with potentially fatal complications.²⁹⁻³¹

The incidence of dengue virus (DENV) infections, a widespread mosquito-borne flaviviral disease, is much higher than the incidence of ZIKV infections, causing approximately 100 million symptomatic infections per year in more than 125 countries.³² The incidence of dengue increased from 1.2 million in 2008 to more than 3.2 million in 2015.³³ The threat of a possible epidemic of dengue fever now exists in Europe, given that autochthonous transmission has been reported in France³⁴ and in other European countries.^{35, 36} Dengue has been a long-standing problem in South Asia. India's first dengue fever epidemic was reported in 1964, spreading in a northward march from the southern peninsula to the foothills of the Himalayas. Extensive epidemics have followed, interspersed with endemic/hyper-endemic years,^{37, 38} and in 2015, New Delhi recorded its worst outbreak since 2006 with over 15,000 cases. Sri Lanka has also been particularly affected; Pro-Med Mail has reported more than 17,600 cases in the first two months of 2017, with 24 deaths.

The four dengue virus serotypes (DENV-1, DENV-2, DENV-3, and DENV-4) exhibit essentially identical tropism (for example, monocytes, macrophages and dendritic cells),^{61, 62} and elicit indistinguishable clinical manifestations. An individual infected with one of the four DENV serotypes usually develops long-lived, protective immunity against the primary strain (homotypic immunity); however, the individual can later be exposed to serotypes other than the one eliciting protective immunity. The low affinity and avidity characteristics of the antibodies elicited by the infection with the first strain can facilitate Antibody Dependent Enhancement (ADE) during subsequent DENV infections by enhancing the targeting of DENV-antibody complexes to Fcγ receptor (FcγR) bearing cells, and subsequent internalization of the virion.⁶⁷⁻⁷³

Yellow fever (YF) virus is also a mosquito-borne flavivirus, causing an acute infection with clinical manifestations ranging from mild non-specific illness to severe disease leading to multiple system organ failure, which is associated with mortality rates up to 50%. Several epidemics of YF have devastated populations in Africa and the Americas since the 17^{th} century.³⁹⁻⁴² An outbreak in the mid-1980s centered in Nigeria developed into a series of epidemics between 1986 and 1991, with 16,230 cases and 3633 deaths.^{→}On much of the African continent—with a population of 1.2 billion—YF is now considered endemic. An outbreak in Brazil is currently ongoing, with 1345 suspected cases (from December 2016 to Feb. 22, 2017), and 215 deaths;⁴⁴ thousands of non-human primates have also succumbed to the disease, raising the specter of extinctions of endangered species.

### SUMMARY OF THE INVENTION

This disclosure describes a subunit vaccine for a flavivirus, methods of making the vaccine, and methods of using the vaccine.

The term “dengue virus” refers to a group of four genetically and antigenically related viruses (DENV-1, DENV-2, DENV-3, and DENV-4).

The term “antibody-dependent enhancement” or “ADE” as used herein refers to phenomena characterized by non-neutralizing (or sub-optimally neutralizing) antibodies that facilitate virus entry into host cells, leading to increased infectivity in the cells. In some embodiments, ADE refers to a significant a detectable increase in viral infection in the presence of an antibody, relative to a preimmune sample or an unrelated antibody.

The term “subunit vaccine” refers to a vaccine that is capable of presenting an antigen from a microbe (for example, a viral particle) to the immune system without introducing the complete microbe.

As used herein “sequence identity” between two polypeptides is determined by comparing the amino acid sequence of one polypeptide to the sequence of a second polypeptide. When discussed herein, whether any particular polypeptide is at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identical to another polypeptide can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman, Advances in Applied Mathematics 2:482-489 (1981), to find the best segment of homology between two sequences. When using BESTFIT or any other sequence alignment program to determine whether a particular sequence is, for example, 95% identical to a reference sequence according to the present invention, the parameters are set, of course, such that the percentage of identity is calculated over the full length of the reference polypeptide sequence and that gaps in homology of up to 5% of the total number of amino acids in the reference sequence are allowed.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (for example, 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

Reference throughout this specification to “one embodiment,” “an embodiment,” “certain embodiments,” or “some embodiments,” etc., means that a particular feature, configuration, composition, or characteristic described regarding the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are provided for the convenience of the reader and should not be used to limit the meaning of the text that follows the heading, unless so specified.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1A shows structures of the component parts of an exemplary adjuvant including the covalent conjugate of an imidazoquinoline TLR7/TLR8 dual agonist [5-(3-(aminomethyl)benzyl)-3-pentylquinolin-2-amine] with hyaluronic acid (also referred to herein as EY-4-143). FIG. 1B shows a schematic of the synthesis of EY-4-143.

FIG. 2A shows the sequence (SEQ ID NO: 23) and crystal structure of the three domains of dengue soluble envelope protein (PDB: 1OK8). Domain III (bold and italicized text; receptor binding, major site for neutralizing Ab, and neutralization escape mutants cluster largely in Domain III). Sequences constituting Domain I (bold and underlined, gray shaded and underlined, and underlined text) and Domain II (plain text; membrane fusion domain) are discontinuous. FIG. 2B shows a model of dengue soluble envelope protein from Dengue-2 (post-fusion conformation). The membrane fusion domain (Domain II) of DENV-Env has been ‘excised,’ and the residual sequences ligated with appropriate spacer glycines. FIG. 2C shows a Raptor X model of the homologous sequences in ZIKV-Env, identified by BLASTP and ligated with appropriate spacer glycines, shows beta-barrel topology.

FIG. 3A shows the sequence of a an MBP-ZIKV E-glycoprotein fusion construct (SEQ ID NO: 24) including the 6His-leader sequence (plain text), MBP sequence (italicized), TEV Protease cleavage site (bold and underlined), and ZIKV E-glycoprotein sequence (shaded in gray). FIG. 3B-FIG. 3F show exemplary results of immunizing rabbits with the MBP-ZIKV E-glycoprotein fusion construct; immune sera neutralized all ZIKV strains in a cytopathic effect (CPE)/cell death assay.

FIG. 4A-FIG. 4D show the absence of antibody-dependent enhancement (ADE) or heterologous protection against DENV-1, DENV-2, DENV-3, or DENV-4 immune sera from rabbits immunized with MBP-ZIKV.

FIG. 5 shows immunodominance of the MBP fragment (SEQ ID NO: 25) (relative to the ZIKV fragment (SEQ ID NO: 1)). The MBP-ZIKV fusion protease was cleaved with TEV protease and probed with preimmune and immune sera from animals immunized with the MBP-ZIKV antigen. A WES instrument (Protein Simple, Bio-Techne, Minneapolis, Minn.) was used for acquiring and analyzing Western blot data.

FIG. 6 shows exemplary in vitro neutralization of ZIKV NR50221 in Immune-2 (and Immune-1) rabbit sera, as described in Example 3, using a cytopathic effect (CPE)/cell death assay plate.

FIG. 7A-FIG. 7B shows exemplary in vitro neutralization of ZIKV NR50221 in Immune-2 (and Immune-1) rabbit sera using a 4G2 immunostain assay plate.

FIG. 8A-FIG. 8E show mass-spectrometric characterization of DENV-1 (FIG. 8A, SEQ ID NO: 2), DENV-2 (FIG. 8B, SEQ ID NO: 3), DENV-3 (FIG. 8C, SEQ ID NO: 4), DENV-4 (FIG. 8 D, SEQ ID NO: 5) and WNV (FIG. 8E, SEQ ID NO: 6) expressed in 10 mg scale. Deconvoluted masses are shown. A Quadrupole Time-of-flight (QTOF) mass spectrometry system (mass accuracy of 20 ppm) was used. Total ion current (TIC) and absorbance profiles indicated a purity of >92%.

FIG. 9A-FIG. 9F show exemplary homotypic neutralizing titers in rabbits immunized with DENV-1, DENV-2, and DENV-4 antigens. Results of cytopathic effect (CPE)/cell death (FIG. 9A-FIG. 9C) and 4G2 immunostain (FIG. 9D-FIG. 9F), are shown.

FIG. 10A shows sequence alignment of the ZIKV, DENV, and WNV antigens (SEQ ID NOs 1-6, respectively, in order of appearance), showing regions of strong homology (gray shading). FIG. 10B-FIG. 10C show exemplary results of a serological cross reactivity matrix. Immune-2 sera from rabbits immunized with the 6 antigens were screened by ELISA for homologous titers. Samples with highest titers were examined for immunoreactivity with all antigens. Significant cross-reactivity was observed. DENV-1 Ag is recognized by anti-DENV-3 and anti-ZIKV antisera. Conversely, anti-DENV-3 antisera recognizes ZIKV antigen. Anti-ZIKV antisera recognizes WNV antigen very strongly.

FIG. 11A-FIG. 11D show the effect of sera from rabbits immunized with DENV-1, DENV-2, DENV-3, DENV-4 antigens on ZIKV replication (non-filled symbols in all panels). Also included as controls were ZIKV antisera (filled symbols). Low level sporadic inhibition of ZIKV (protection) and no antibody dependent enhancement (ADE) was observed. No significant differences between pre-immune and immune sera were noted.

FIG. 12A-FIG. 12D shows heterotypic protection using the 4G2 immunostain assay method, and no ADE was observed in DENV/WNV antisera. Cohorts of rabbits were immunized separately with ZIKV, DENV-1, DENV-2, DENV-3, DENV-4, and WNV antigens. In vitro challenge with DENV-1 or DENV-2 strains show prominent heterologous protection in DENV-3 antisera to DENV-1 challenge (FIG. 12A). Means of duplicates are shown.

FIG. 13A-FIG. 13D show heterotypic protection using the 4G2 immunostain assay method, and no ADE is observed in DENV/WNV antisera. Cohorts of rabbits were immunized separately with ZIKV, DENV-1, DENV-2, DENV-3, DENV-4, and WNV antigens. Means of duplicates are shown.

FIG. 14. Left panel. The addition of the adjuvant, EY-4-143 to a mixture of DENV-1, -2, -3, and -4 antigens results in a shift from a retention time corresponding to ˜12 kDa to the void volume in size exclusion chromatography (SEC) (Sephacryl Hi-Prep S200). Right panel. The addition of EY-4-143 to 15N-labeled ZIKV antigen results in specific chemical shift perturbations in heteronuclear single quantum correlation (HSQC) spectra (arrows).

FIG. 15A-FIG. 15E show alignments of the other antigenic sequences of Table 4 with the sequence of the ZIKV antigen sequence of Table 4. “Identities” indicates the percentage (%) of identical residues; “Positives” indicates the percentage of residues of similar property (including the identical residues); “Gaps” indicate missing or addition residues. FIG. 15A discloses SEQ ID NOs 26 and 2, respectively, in order of appearance. FIG. 15B discloses SEQ ID NOs 1 and 3 respectively, in order of appearance. FIG. 15C discloses SEQ ID NOs 26 and 27, respectively, in order of appearance. FIG. 15D discloses SEQ ID NOs 26 and 5, respectively, in order of appearance. FIG. 15E discloses SEQ ID NOs 1 and 6, respectively, in order of appearance.

FIG. 16A-FIG. 16C show alignments of DENV-2, DENV-3, and DENV-4 antigens of Table 4 with the sequence of the DENV-1 antigen of Table 4. “Identities” indicates the percentage (%) of identical residues; “Positives” indicates the percentage of residues of similar property (including the identical residues); “Gaps” indicate missing or addition residues. FIG. 16A discloses SEQ ID NOs 2 and 28, respectively, in order of appearance. FIG. 16B discloses SEQ ID NOs 2 and 27, respectively, in order of appearance. FIG. 16C discloses SEQ ID NOs 2 and 5, respectively, in order of appearance.

FIG. 17A-FIG. 17B show ZIKV neutralizing titers in animals vaccinated with MBP-2-ZIKV (FIG. 17A) or Hexavalent MBP2-ZIKV/WNV/DENV-(1-4) (FIG. 17B), measured in a CPE assay using ZIKV-Thai as a representative clinical isolate.

FIG. 18A-FIG. 18C show DENV-1 neutralizing titers in animals vaccinated with MBP-2-DENV-1 (FIG. 18A), Tetravalent MBP2-DENV-(1-4) (FIG. 18B), or Hexavalent MBP2-ZIKV/WNV/DENV-(1-4) (FIG. 18C), as measured using GFP-expressing recombinant viral particles (DENV-1 GFP-RVP (WestPac).

FIG. 19A-FIG. 19C show DENV-2 neutralizing titers in animals vaccinated with MBP-2-DENV-2 (FIG. 19A), Tetravalent MBP2-DENV-(1-4) (FIG. 19B), or Hexavalent MBP2-ZIKV/WNV/DENV-(1-4) (FIG. 19C), as measured using DENV-2 (NR43280, DENV-2/US/BID-V594/2006, Puerto Rico), in a CPE assay.

FIG. 20A-FIG. 20C show DENV-3 neutralizing titers in animals vaccinated with MBP-2-DENV-3 (FIG. 20A), Tetravalent MBP2-DENV-(1-4) (FIG. 20B), or Hexavalent MBP2-ZIKV/WNV/DENV-(1-4) (FIG. 20C) as measured using GFP-expressing recombinant viral particles (DENV-3 GFP-RVP (CH3489).

FIG. 21A-FIG. 21C show DENV-4 neutralizing titers in animals vaccinated with MBP-2-DENV-4 (FIG. 21A), Tetravalent MBP2-DENV-(1-4) (FIG. 21B), or Hexavalent MBP2-ZIKV/WNV/DENV-(1-4) (FIG. 21C), as measured using GFP-expressing recombinant viral particles (DENV-4 GFP-RVP (TVP360).

FIG. 22A-FIG. 22B show WNV neutralizing titers in animals vaccinated with MBP-2-WNV (FIG. 22A) or Hexavalent MBP2-ZIKV/WNV/DENV-(1-4) (FIG. 22B), as measured using GFP-expressing recombinant viral particles (WNV GFP-RVP).

FIG. 23 shows seroconversion (ZIKV-specific IgG titers) with three different immunization regimes (as shown in Table 8) including SGp(L)-ZIKV. SGp(L) fusion constructs of ZIKV, DENV-1, DENV-2, DENV-3, DENV-4, and WNV were used in conjunction with the adjuvant (HA-conjugate). Animals received all six antigens in one site (Cohort A), or DENV (1-3) antigens on one flank, and DENV-4, WNV, and ZIKV on the other flank (Cohort B). Cohort C received DENV-1, DENV-2, DENV-3, WNV and ZIKV antigens on one flank, and DENV-4 antigen on the other flank. These results show equivalence between all three immunization regimes.

FIG. 24 shows seroconversion (WNV-specific IgG titers) with three different immunization regimes (as shown in Table 8) including SGp(L)-WNV. SGp(L) fusion constructs of ZIKV, DENV-1, DENV-2, DENV-3, DENV-4, and WNV were used in conjunction with the adjuvant (HA-conjugate). Animals received all six antigens in one site (Cohort A), or DENV (1-3) antigens on one flank, and DENV-4, WNV, and ZIKV on the other flank (Cohort B). Cohort C received DENV-1, DENV-2, DENV-3, WNV and ZIKV antigens on one flank, and DENV-4 antigen on the other flank. These results show equivalence between all three immunization regimes.

FIG. 25 shows seroconversion (DENV-1-specific IgG titers) with three different immunization regimes (as shown in Table 8) including SGp(L)-DENV-1 antigen. SGp(L) fusion constructs of ZIKV, DENV-1, DENV-2, DENV-3, DENV-4, and WNV were used in conjunction with the adjuvant (HA-conjugate). Animals received all six antigens in one site (Cohort A), or DENV (1-3) antigens on one flank, and DENV-4, WNV, and ZIKV on the other flank (Cohort B). Cohort C received DENV-1, DENV-2, DENV-3, WNV and ZIKV antigens on one flank, and DENV-4 antigen on the other flank. These results show equivalence between all three immunization regimes.

FIG. 26 shows seroconversion (DENV-2-specific IgG titers) with three different immunization regimes (as shown in Table 8) including SGp(L)-DENV-2 antigen. SGp(L) fusion constructs of ZIKV, DENV-1, DENV-2, DENV-3, DENV-4, and WNV were used in conjunction with the adjuvant (HA-conjugate). Animals received all six antigens in one site (Cohort A), or DENV (1-3) antigens on one flank, and DENV-4, WNV, and ZIKV on the other flank (Cohort B). Cohort C received DENV-1, DENV-2, DENV-3, WNV and ZIKV antigens on one flank, and DENV-4 antigen on the other flank. These results show equivalence between all three immunization regimes.

FIG. 27 shows seroconversion (DENV-3-specific IgG titers) with three different immunization regimes (as shown in Table 8) including SGp(L)-DENV-3 antigen. SGp(L) fusion constructs of ZIKV, DENV-1, DENV-2, DENV-3, DENV-4, and WNV were used in conjunction with the adjuvant (HA-conjugate). Animals received all six antigens in one site (Cohort A), or DENV (1-3) antigens on one flank, and DENV-4, WNV, and ZIKV on the other flank (Cohort B). Cohort C received DENV-1, DENV-2, DENV-3, WNV and ZIKV antigens on one flank, and DENV-4 antigen on the other flank. These results show equivalence between all three immunization regimes.

FIG. 28 shows seroconversion (DENV-4-specific IgG titers) with three different immunization regimes (as shown in Table 8) including SGp(L)-DENV-4 antigen. SGp(L) fusion constructs of ZIKV, DENV-1, DENV-2, DENV-3, DENV-4, and WNV were used in conjunction with the adjuvant (HA-conjugate). Animals received all six antigens in one site (Cohort A), or DENV (1-3) antigens on one flank, and DENV-4, WNV, and ZIKV on the other flank (Cohort B). Cohort C received DENV-1, DENV-2, DENV-3, WNV and ZIKV antigens on one flank, and DENV-4 antigen on the other flank. These results show equivalence between all three immunization regimes.

FIG. 29 shows neutralizing titers for ZIKV in animals vaccinated as described in Example 7 SGp(L) fusion constructs of ZIKV, DENV-1, DENV-2, DENV-3, DENV-4, and WNV were used in conjunction with the adjuvant (HA-conjugate). Animals received all six antigens in one site (Cohort A), or DENV (1-3) antigens on one flank, and DENV-4, WNV, and ZIKV on the other flank (Cohort B). Cohort C received DENV-1, DENV-2, DENV-3, WNV and ZIKV antigens on one flank, and DENV-4 antigen on the other flank.

FIG. 30 shows neutralizing titers for WNV in animals vaccinated as described in Example 7. SGp(L) fusion constructs of ZIKV, DENV-1, DENV-2, DENV-3, DENV-4, and WNV were used in conjunction with the adjuvant (HA-conjugate). Animals received all six antigens in one site (Cohort A), or DENV (1-3) antigens on one flank, and DENV-4, WNV, and ZIKV on the other flank (Cohort B). Cohort C received DENV-1, DENV-2, DENV-3, WNV and ZIKV antigens on one flank, and DENV-4 antigen on the other flank.

FIG. 31 shows neutralizing titers for DENV-1 in animals vaccinated as described in Example 7. SGp(L) fusion constructs of ZIKV, DENV-1, DENV-2, DENV-3, DENV-4, and WNV were used in conjunction with the adjuvant (HA-conjugate). Animals received all six antigens in one site (Cohort A), or DENV (1-3) antigens on one flank, and DENV-4, WNV, and ZIKV on the other flank (Cohort B). Cohort C received DENV-1, DENV-2, DENV-3, WNV and ZIKV antigens on one flank, and DENV-4 antigen on the other flank.

FIG. 32 shows neutralizing titers for DENV-2 in animals vaccinated as described in Example 7. SGp(L) fusion constructs of ZIKV, DENV-1, DENV-2, DENV-3, DENV-4, and WNV were used in conjunction with the adjuvant (HA-conjugate). Animals received all six antigens in one site (Cohort A), or DENV (1-3) antigens on one flank, and DENV-4, WNV, and ZIKV on the other flank (Cohort B). Cohort C received DENV-1, DENV-2, DENV-3, WNV and ZIKV antigens on one flank, and DENV-4 antigen on the other flank.

FIG. 33 shows neutralizing titers for DENV-3 in animals vaccinated as described in Example 7. SGp(L) fusion constructs of ZIKV, DENV-1, DENV-2, DENV-3, DENV-4, and WNV were used in conjunction with the adjuvant (HA-conjugate). Animals received all six antigens in one site (Cohort A), or DENV (1-3) antigens on one flank, and DENV-4, WNV, and ZIKV on the other flank (Cohort B). Cohort C received DENV-1, DENV-2, DENV-3, WNV and ZIKV antigens on one flank, and DENV-4 antigen on the other flank.

FIG. 34 shows neutralizing titers for DENV-4 in animals vaccinated as described in Example 7. SGp(L) fusion constructs of ZIKV, DENV-1, DENV-2, DENV-3, DENV-4, and WNV were used in conjunction with the adjuvant (HA-conjugate). Animals received all six antigens in one site (Cohort A), or DENV (1-3) antigens on one flank, and DENV-4, WNV, and ZIKV on the other flank (Cohort B). Cohort C received DENV-1, DENV-2, DENV-3, WNV and ZIKV antigens on one flank, and DENV-4 antigen on the other flank. These results show equivalence between all three immunization regimes.

### DETAILED DESCRIPTION OF ILLUSTRATIVE EMBODIMENTS

This disclosure describes a subunit vaccine for a flavivirus, methods of making the vaccine, and methods of using the vaccine. In some embodiments, the flavivirus is a mosquito-borne flavivirus. In some embodiments, the flavivirus includes at least one of Zika virus (ZIKV), dengue virus (DENV), Yellow Fever (YF) virus, and West Nile Virus (WNV). DENV can include any one of the DENV serotypes (for example, DENV-1, DENV-2, DENV-3, and DENV-4).

### Vaccine

In preferred embodiments, the vaccine described herein is preferably a subunit vaccine—that is, a vaccine that includes a viral antigen that is capable of being presented to the immune system without introducing an entire viral particle.

### Antigen

The vaccines described herein include at least one antigen. Preferably, when the antigen is presented to the immune system, a response is elicited against at least one flavivirus. In some embodiments, the flavivirus is a mosquito-borne flavivirus. In some embodiments, the flavivirus includes at least one of Zika virus (ZIKV), dengue virus (DENV), Yellow Fever (YF) virus, and West Nile Virus (WNV). DENV can include any one of the DENV serotypes (for example, DENV-1, DENV-2, DENV-3, and DENV-4).

In some embodiments, the antigen may include a sequence set forth in Table 4 (SEQ ID NOs 1-6), a sequence set forth in Table 5A (SEQ ID NOs 7-12), or a sequence having at least 50%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to at least one of the sequences of Table 4 (SEQ ID NOs 1-6) or Table 5A (SEQ ID NOs 7-12).

In some embodiments, the vaccine includes more than one antigen including, for example, more than one flavivirus antigen. For example, the vaccine may include a mixture of DENV-1/-2/-3/-4 antigens. In some embodiments, a vaccine may include a mixture of DENV-1/-2/-3/-4 antigens, ZIKV antigen, and WNV antigen. In some embodiments, one or more of the antigens included in the vaccine has a sequence as shown in Table 4 or Table 5A. In some embodiments, one or more of the antigens included in the vaccine has at least 50%, 60%, 65%, 70%, 75%, 80%, 81%, 82%, 83%, 84%, 85%, 86%, 87%, 88%, 89%,90%, 91%, 92%, 93%, 94%, 95%, 96%, 97%, 98%, or 99% sequence identity to at least one of the sequences of Table 4 (SEQ ID NOs 1-6) or Table 5A (SEQ ID NOs 7-12).

In some embodiments, the vaccine preferably includes more than one antigen (including, for example, an antigen from ZIKV, WNV, and/or any one of the DENV serotypes) and elicits a response against at least one flavivirus, at least two flaviviruses, or at least three flaviviruses. In some embodiments, the vaccine does not elicit antibody-dependent enhancement. In some embodiments, the vaccine does not elicit immune interference.

For example, in some embodiments, the vaccine may include two of the sequences of Table 4 (SEQ ID NOs 1-6) and Table 5A (SEQ ID NOs 7-12), three of the sequences of Table 4 (SEQ ID NOs 1-6) and Table 5A (SEQ ID NOs 7-12), four of the sequences of Table 4 (SEQ ID NOs 1-6) and Table 5A (SEQ ID NOs 7-12), five of the sequences of Table 4 (SEQ ID NOs 1-6) and Table 5A (SEQ ID NOs 7-12), all of the sequences of Table 4 (SEQ ID NOs 1-6), or all of the sequences of Table 5A (SEQ ID NOs 7-12).

In some embodiments, the vaccine may further include a tag, and, in some embodiments, the antigen may be operably linked to the tag. A tag may include a maltose binding protein (MBP), a small ubiquitin-like modifier (SUMO) (Panavas et al. Methods Mol Biol. 2009;497:303-17), a Glutathione S-transferase (GST), a Streptococcal G protein (SGp), etc., or combinations and/or portions thereof.

As used herein, the term “operably linked” refers to direct or indirect covalent linking. Thus, two domains that are operably linked may be directly covalently coupled to one another.

Conversely, the two operably linked domains may be connected by mutual covalent linking to an intervening moiety (for example, and flanking sequence). Two domains may be considered operably linked if, for example, they are separated by the third domain, with or without one or more intervening flanking sequences.

For example, in some embodiments, the tag may include at least one of the sequences of Table 5B (SEQ ID NOs 13-14). In some embodiments, the antigen may include a sequence having at least 50%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to at least one of the sequences of Table 5B (SEQ ID NOs 13-14).

In some embodiments, the tag may be selected for its binding to albumin (human, non-human primate, pig, rabbit, rat, mouse). In some embodiments, the tag may be selected for its lack of binding to immunoglobulins. Without wishing to be bound by theory, it is believed that the binding to albumin facilitates delivery of the antigen to the lymph nodes while the abrogation of binding to immunoglobulins may obviate problems with affinity maturation of the resultant antibody response. For example, as described in Example 7, a portion of SGp may be used that binds to albumin but not to immunoglobulins.

### Methods of Making the Vaccine

In another aspect this disclosure describes methods of making the vaccine including, for example, making a vaccine including a tag and/or a protease cleavage site.

### Tag

In some embodiments, the sequence encoding the antigen may be linked to a sequence (for example, a gene) encoding a tag such that the resulting antigen is operably linked to a tag. In some embodiments, the sequence encoding a tag may include a maltose binding protein (MBP) sequence, a small ubiquitin-like modifier (SUMO) sequence (Panavas et al. Methods Mol Biol. 2009;497:303-17), a Glutathione S-transferase (GST) sequence, a Streptococcal G protein (SGp) sequence, etc., or combinations and/or portions thereof.

For example, in some embodiments, the tag may include a sequence that encodes a protein having at least one of the sequences of Table 5B (SEQ ID NOs 13-14). In some embodiments, the tag may include a sequence that encodes a protein having at least 50%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to at least one of the sequences of Table 5B (SEQ ID NOs 13-14).

### Protease Cleavage Site

In some embodiments, a protease cleavage site may be included between the sequence encoding the antigen and the sequence encoding the tag. Any suitable protease cleavage site may be used. In some embodiments, the protease cleavage site can include, for example, at least one of a TEV Protease cleavage site, an Enteropeptidase cleavage site, a thrombin cleavage site, a Factor Xa cleavage site, and a Rhinovirus 3C Protease cleavage site.

### Methods of Using the Vaccine

### Adjuvant

In some embodiments, the subunit vaccine described herein is preferably administered with an adjuvant. Any suitable adjuvant may be used. An adjuvant may include, for example, a suspensions of mineral (alum, aluminum hydroxide, aluminum phosphate) onto which antigen is adsorbed; an emulsion, including water-in-oil, and oil-in-water (and variants thereof, including double emulsions and reversible emulsions); a liposaccharides; a lipopolysaccharide; an immunostimulatory nucleic acid (such as a CpG oligonucleotide); a liposome; a Toll-like Receptor (TLR) agonist (for example, TLR2, TLR4, TLR7/8 and TLR9 agonists); and various combinations thereof.

In some embodiments, the adjuvant is preferably a TLR agonist.

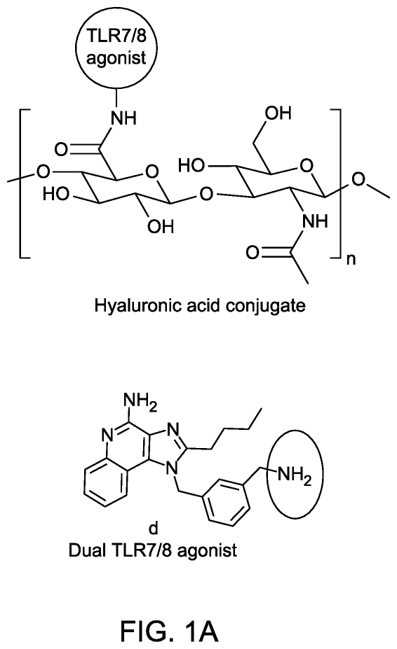

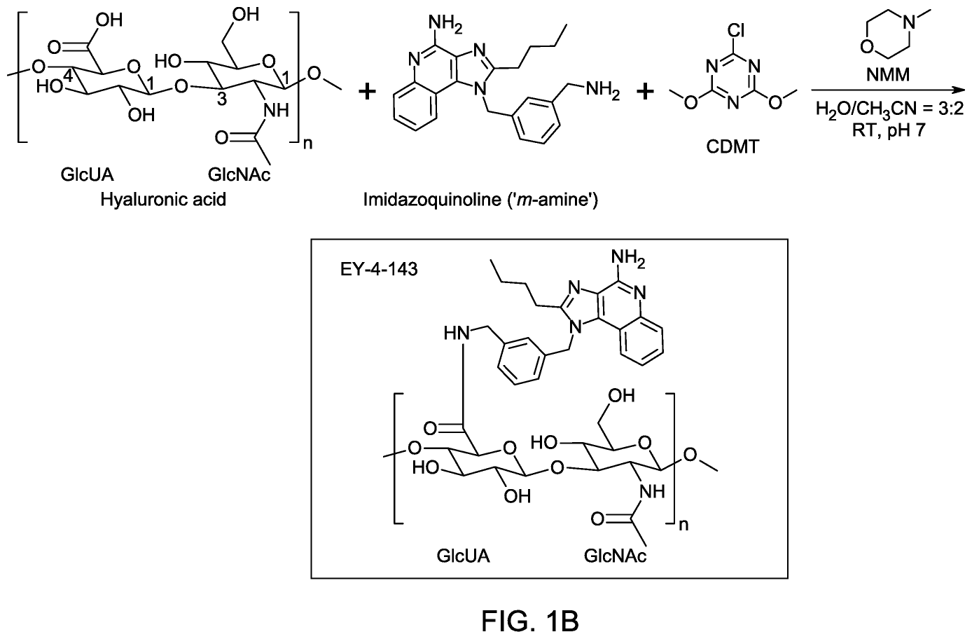

In some embodiments, the adjuvant is preferably a covalent conjugate of an imidazoquinoline TLR7/TLR8 dual agonist [5-(3-(aminomethyl)benzyl)-3-pentylquinolin-2-amine] with hyaluronic acid (also referred to herein as EY-4-143) (FIG. 1). In some embodiments, a triazine-activated amidation strategy using 2-chloro-4,6-dimethoxy-1,3,5-triazine (CDMT)^{180, 181} may be used for formation of the conjugate.

EY-4-143 is inert in vitro. It is inactive in TLR-7 and -8 primary assays, and is also silent in cytokine induction assays using either human whole blood or PBMCs; EY-4-143, however, is a potent adjuvant. A depot effect for EY-4-143 has been observed: at the site of injection as well as in the draining lymph node, there is a low-level, but sustained release of the TLR7/8 agonist. A comparison of equivalent doses of the free unconjugated imidazoquinoline with EY-4-143 in a reporter mouse model indicates that the systemic exposure of the EY-4-143 is very small relative to the unconjugated imidazoquinolines, signifying very low reactogenic potential and a high margin of safety. Without wishing to be bound by theory, the superior adjuvantic effects of EY-4-143 in combination with the vaccines disclosed herein are believed to be attributable, at least in part, to this depot effect.

### Pharmaceutical Compositions

The present disclosure provides a pharmaceutical composition that includes a vaccine as described herein, and a pharmaceutically acceptable carrier. The vaccine is formulated in a pharmaceutical composition and then, in accordance with the method of the invention, administered to a vertebrate, particularly mammal, such as a human patient, primate, research animal, or domesticated animal, in a variety of forms adapted to the chosen route of administration. The formulations include those suitable for oral, rectal, vaginal, topical, nasal, ophthalmic, or parenteral (including subcutaneous, intramuscular, intraperitoneal, and intravenous) administration.

The pharmaceutically acceptable carrier can include, for example, an excipient, a diluent, a solvent, an accessory ingredient, a stabilizer, a protein carrier, or a biological compound. Non-limiting examples of a protein carrier includes keyhole limpet hemocyanin (KLH), bovine serum albumin (BSA), ovalbumin, or the like. Non-limiting examples of a biological compound which can serve as a carrier include a glycosaminoglycan, a proteoglycan, and albumin. The carrier can be a synthetic compound, such as dimethyl sulfoxide or a synthetic polymer, such as a polyalkyleneglycol. Ovalbumin, human serum albumin, other proteins, polyethylene glycol, or the like can be employed as the carrier. In some embodiments, the pharmaceutically acceptable carrier includes at least one compound that is not naturally occurring or a product of nature.

The formulations can be conveniently presented in unit dosage form and can be prepared by any of the methods well-known in the art of pharmacy. In some embodiments, a method includes the step of bringing the vaccine into association with a pharmaceutical carrier. In general, the formulations are prepared by uniformly and intimately bringing the active compound into association with a liquid carrier, a finely divided solid carrier, or both, and then, if necessary, shaping the product into the desired formulations.

Formulations of the present disclosure suitable for oral administration can be presented as discrete units such as tablets, troches, capsules, lozenges, wafers, or cachets, each containing a predetermined amount of the vaccine as a powder or granules, as liposomes, or as a solution or suspension in an aqueous liquor or non-aqueous liquid such as a syrup, an elixir, an emulsion, or a draught. The tablets, troches, pills, capsules, and the like can also contain one or more of the following: a binder such as gum tragacanth, acacia, corn starch or gelatin; an excipient such as dicalcium phosphate; a disintegrating agent such as corn starch, potato starch, alginic acid, and the like; a lubricant such as magnesium stearate; a sweetening agent such as sucrose, fructose, lactose, or aspartame; and a natural or artificial flavoring agent. When the unit dosage form is a capsule, it can further contain a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, sugar, and the like. A syrup or elixir can contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The vaccine can be incorporated into preparations and devices in formulations that may or may not be designed for sustained release.

Formulations suitable for parenteral administration conveniently include a sterile aqueous preparation of the vaccine, or dispersions of sterile powders of the vaccine, which are preferably isotonic with the blood of the recipient. Parenteral administration a vaccine (e. g., through an I. V. drip) is one form of administration. Isotonic agents that can be included in the liquid preparation include sugars, buffers, and sodium chloride. Solutions of the vaccine can be prepared in water, optionally mixed with a nontoxic surfactant. Dispersions of the vaccine can be prepared in water, ethanol, a polyol (such as glycerol, propylene glycol, liquid polyethylene glycols, and the like), vegetable oils, glycerol esters, and mixtures thereof. The ultimate dosage form is sterile, fluid, and stable under the conditions of manufacture and storage. In some embodiments, the necessary fluidity can be achieved, for example, by using liposomes, by employing the appropriate particle size in the case of dispersions, or by using surfactants. Sterilization of a liquid preparation can be achieved by any convenient method that preserves the bioactivity of the vaccine, including, for example, by filter sterilization. Preferred methods for preparing powders include vacuum drying and freeze drying of the sterile injectable solutions. Subsequent microbial contamination can be prevented using various antimicrobial agents, for example, antibacterial, antiviral and antifungal agents including parabens, chlorobutanol, phenol, sorbic acid, thimerosal, and the like. Absorption of the vaccine over a prolonged period may be achieved by including agents for delaying, for example, aluminum monostearate and gelatin.

Nasal spray formulations include purified aqueous solutions of the vaccine with preservative agents and isotonic agents. Such formulations may preferably be adjusted to a pH and isotonic state compatible with the nasal mucous membranes. Formulations for rectal or vaginal administration can be presented as a suppository with a suitable carrier such as cocoa butter, or hydrogenated fats or hydrogenated fatty carboxylic acids. Ophthalmic formulations may prepared by a similar method to the nasal spray, except that the pH and isotonic factors are preferably adjusted to match that of the eye. Topical formulations include the vaccine dissolved or suspended in one or more media such as mineral oil, petroleum, polyhydroxy alcohols, or other bases used for topical pharmaceutical formulations. Topical formulations may be provided in the form of a bandage, wherein the formulation is incorporated into a gauze or other structure and brought into contact with the skin.

### Administration

A subunit vaccine, as described herein, can be administered to a subject alone or in a pharmaceutical composition that includes the vaccine and a pharmaceutically acceptable carrier. The vaccine may be administered to a vertebrate, more preferably a mammal, such as a human patient, a companion animal, or a domesticated animal, in an amount effective to produce the desired effect. The vaccine may be administered in a variety of routes, including orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form.

The formulations may be administered as a single dose or in multiple doses. Useful dosages of the vaccine may be determined by comparing their in vitro activity and the in vivo activity in animal models. Methods for extrapolation of effective dosages in rabbits, mice, primates, and other animals, to humans are known in the art.

Dosage levels of the vaccine in the pharmaceutical compositions of this disclosure can be varied so as to obtain an amount of the vaccine which is effective to achieve the desired therapeutic response for a particular subject, composition, and mode of administration, without being toxic to the subject. The selected dosage level will depend upon a variety of factors including the route of administration, the time of administration, the adjuvant being employed, the duration of the treatment, other drugs, compounds and/or materials used in combination with the vaccine, the age, sex, weight, condition, general health and prior medical history of the subject being treated, and like factors well known in the medical arts.

Dosages and dosing regimens that are suitable for other vaccines may likewise be suitable for therapeutic or prophylactic administration of the vaccines described herein.

A physician or veterinarian having ordinary skill in the art can readily determine and prescribe the effective amount of the vaccine and/or pharmaceutical composition required.

### Exemplary Embodiments

1. A subunit vaccine for a flavivirus, the vaccine comprising an antigen, the antigen comprising a sequence having at least 80% sequence identity to one of SEQ ID NO:1 to SEQ ID NO:12.
2. The subunit vaccine of Embodiment 1, wherein the antigen comprises a sequence having at least 90% sequence identity to one of SEQ ID NO:1 to SEQ ID NO:12.
3. The subunit vaccine of either of Embodiments 1 or 2, wherein the antigen comprises a sequence comprising one of SEQ ID NO:1 to SEQ ID NO:12.
4. A subunit vaccine for a flavivirus, the vaccine comprising an antigen consisting of a sequence having at least 80% sequence identity to one of SEQ ID NO:1 to SEQ ID NO:12.
5. The subunit vaccine of Embodiment 4, the antigen consisting of a sequence having at least 90% sequence identity to one of SEQ ID NO:1 to SEQ ID NO:12.
6The subunit vaccine of Embodiments 4 or 5, the antigen consisting of a sequence selected from the group consisting of one of SEQ ID NO:1 to SEQ ID NO:12.
7. The subunit vaccine of any one of Embodiments 1 to 6, wherein the flavivirus comprises at least one of Zika virus (ZIKV), dengue virus (DENV), Yellow Fever (YF) virus, and West Nile Virus (WNV).
8. The subunit vaccine of Embodiment 7, wherein DENV comprises at least one of DENV-1, DENV-2, DENV-3, and DENV-4.
9. The subunit vaccine of any one of Embodiments 1 to 6, wherein the vaccine further comprises a tag, wherein the tag is fused to the antigen.
10. The subunit vaccine of Embodiment 9, wherein the tag comprises a maltose binding protein (MBP), a small ubiquitin-like modifier (SUMO), a Glutathione S-transferase (GST), a Streptococcal G protein (SGp), or combinations and/or portions thereof
11. The subunit vaccine of Embodiment 9 or 10, wherein the tag comprises SEQ ID NO:13.
12. The subunit vaccine of any of Embodiments 9 to 11, wherein the tag comprises SEQ ID NO:14.
13. A pharmaceutical composition comprising:

the subunit vaccine of any one of Embodiments 1 to 12, and

a pharmaceutically acceptable carrier.

14. The composition of Embodiment 13, the composition further comprising an adjuvant.
15. The composition of Embodiment 14, the adjuvant comprising hyaluronic acid.
16. The composition of Embodiments 14 or 15, the adjuvant comprising a TLR agonist.
17. The composition of Embodiment 16, the TLR agonist comprising at least one of a TLR 7 and a TLR 8 agonist.
18. The composition of Embodiment 16, the TLR agonist comprising [5-(3-(aminomethyl)benzyl)-3-pentylquinolin-2-amine].
19. The composition of any one of Embodiments 13 to 18, the adjuvant comprising a covalent conjugate of hyaluronic acid and a TLR agonist.
20. The composition of any one of Embodiments 13 to 19, the adjuvant comprising

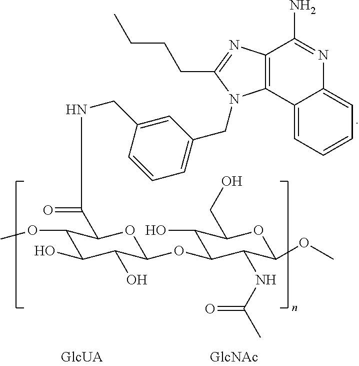

21. A method of making the subunit vaccine of any one of Embodiments 1 to 12.
22. The method of Embodiment 21, wherein the method comprises expressing a construct comprising a sequence encoding the antigen, wherein the resulting antigen is operably linked to a tag.
23. The method of Embodiment 21 or 22, wherein the the method comprises expressing a construct comprising the antigen and a sequence encoding a tag, wherein the construct further comprises a protease cleavage site between the sequence encoding the antigen and the sequence encoding the tag.
24. The method of Embodiment 23, wherein the protease cleavage site comprises a TEV protease cleavage site.
25. The method of any of Embodiments 21 to 24, wherein the tag comprises a maltose binding protein (MBP), a small ubiquitin-like modifier (SUMO), a Glutathione S-transferase (GST), a Streptococcal G protein (SGp), or combinations and/or portions thereof.
26. The method of any of Embodiments 21 to 25, wherein the tag comprises SEQ ID NO:13.
27. The method of any of Embodiments 21 to 26, wherein the tag comprises SEQ ID NO:14.
28. The method of any one of Embodiments 21 to 27, the method further comprising cleaving the tag from the antigen.
29. The method of Embodiment 28, wherein the tag is cleaved from the antigen by TEV protease.
30. A method comprising administering the subunit vaccine of any one of Embodiments 1 to 12.
31. A method comprising administering the composition of any one of Embodiments 13 to 20.
32. The method of Embodiment 30 or 31, wherein the subunit vaccine does not elicit antibody-dependent enhancement.
33. The method of Embodiment 30 or 31, wherein the subunit vaccine does not elicit immune interference.

The present invention is illustrated by the following examples. It is to be understood that the particular examples, materials, amounts, and procedures are to be interpreted broadly in accordance with the scope and spirit of the invention as set forth herein.

### EXAMPLES

### Materials & Methods

### Expression Vectors

The vectors used were modified from the ligation independent cloning (LIC) vector pTBSG described in Qin et al. (BMC Biotechnol. 2008, 8:51). The maltose binding protein (MBP) gene was amplified by PCR using the sense primer 5′-GGACTAGTAAAATCGAAGAAGGTAAACTG-3′ (SEQ ID NO: 16) and anti-sense primer 5′-CGGGGTACCAGTCTG CGCGTCTTTCAG-3′ (SEQ ID NO: 17). The PCR products were digested with restriction enzymes SpeI and KpnI, and then ligated into the pTBSG vector digested with the same enzymes.

### Cloning

Target genes were codon optimized, synthesized (Integrated Gene Technologies, Coralville, Iowa), and amplified by PCR using a pair of primers in which the sense primer began with the sequence 5′-TACTTCCAATCCAATGCA-3′ (SEQ ID NO: 18) followed by the target gene and the anti-sense primer began with the sequence 5′-TTATCCACTTCCAATG-3′ (SEQ ID NO: 19) followed by the complement of a stop codon and the C-terminus of the target gene. The volume of a typical reaction mixture was 50 μL, and the product was purified using a YM-30 spin column (Microcon, Inc.) and recovered in 50 μL of buffer including 50 mM Tris (pH 8.0) and 1 mM EDTA. The vector (with or without fusion partners) was digested with Ssp1 for two hours and applied to DNA agarose electrophoresis. The band corresponding to the cleaved vector was carefully sliced and recovered from the gel using the QIAGEL extraction Kit (Qiagen, Hilden, Germany) and then treated with T4 DNA polymerase (Novagen, LIC quality, EMD Millipore, Billerica, Mass.) in the presence of dGTP. The insert was treated with dCTP and T4 DNA polymerase at room temperature for 30 minutes then heated at 75° C. for 20 minutes to stop the reaction. Annealing was carried out simply by mixing 1 μL of the digested vector, 2 μL of the insert, and 1 μL of EDTA (25 mM, pH 8.0) and incubated at room temperature for 5 minutes. The annealed plasmid was transformed into DH5α competent cells. Positive clones were screened by PCR and then sequenced. Cloned genes were transformed into the expression host, BL21(DE3)-pRARE (KanPro, Inc., Lawrence, Kans.).

### Protein Expression Screening

E. coli cells harboring the expression vector were grown on LB agar plates containing 100 μg/mL ampicillin and 34 μg/mL chloramphenicol. A single clone was picked and inoculated into 3 mL LB media for overnight growth. 100 μL of the overnight culture was then inoculated into 10 mL LB media, and the expression was induced with the addition of 0.4 mM IPTG when OD₆₀₀ reached 0.6. The culture was grown for an additional 4 hours at 37° C., or overnight at 17° C. Cells were harvested by centrifugation at 4500 g for 15 min at 4° C., resuspended in 1 mL lysis buffer (10 mM Tris-HCl, pH 8.0, 0.5 M NaCl) and lysed by sonication (Sonic Dismembrator, Model 100, Fischer Scientific, Inc.) three times (15 sec each). The lysate was fractionated by centrifugation for 20 min at 10,000 g. The supernatant normally contained soluble proteins and fragmented membranes, while the pellet consisted of insoluble proteins (inclusion body fraction). The supernatant was subjected to ultracentrifugation at 100,000 g for 45 min at 8° C. to separate the membrane and soluble protein fractions. The soluble, insoluble and membrane fractions were adjusted to the same volume with lysis buffer, and then 20 μL of each was mixed with 20 μL sample buffer (2×) and 10 μL was loaded on 12% MOPS SDS-PAGE gels followed by either Coomassie staining. Control experiments were performed under the same experimental conditions without IPTG induction.

### Small Scale Purification of Soluble Fractions of the Expressed Antigens

Small-scale purification of each of the target subunit immunogen proteins was studied using a 10 mL culture described as above. Each construct was expressed in 10 mL cultures as described above. The soluble fraction was collected in 50 mM Tris-HCl, pH 8, 500 mM NaCl at 4° C. After centrifugation at 195,000 rpm for 30 min at 4° C., the supernatant was mixed with 100 μL Ni²⁺-NTA resin (Sigma) which was pre-equilibrated with the wash buffer (50 mM Tris-HCl, pH 8, 500 mM NaCl). After incubation at 4° C. overnight with gentle shaking, the resin was extensively washed with the wash buffer and then eluted with the elution buffer (50 mM Tris-HCl, pH 8, 500 mM NaCl, and 250 mM imidazole). Insoluble protein was dissolved in the buffer dissolving buffer (50 mM Tris-HCl, pH 8, 500 mM NaCl, 6 M urea), incubate with NiNTA resin, washed and eluted in 50 mM Tris-HCl, pH 8, 500 mM NaCl, 250 mM imidazole.

### Adjuvant

The adjuvant used herein is a covalent conjugate of an imidazoquinoline TLR7/TLR8 dual agonist [5-(3-(aminomethyl)benzyl)-3-pentylquinolin-2-amine] with hyaluronic acid (also referred to herein as EY-4-143) (FIG. 1). The triazine-activated amidation strategy using 2-chloro-4,6-dimethoxy-1,3,5-triazine (CDMT)^{180, 181} was used for formation of the conjugate.

### Standardized Rabbit Model of Immunogenicity

Pre-immune test-bleeds are first obtained from adult New Zealand White rabbits via venipuncture of the marginal vein of the ear on Day 1. The rabbits are immunized intramuscularly in the flank region with (a) 10 μg of antigen in 0.2 mL saline (n=4 for non-adjuvanted, antigen+saline control cohort), or (b) 10 μg of antigen plus 100 μg of adjuvant in 0.2 mL saline (n=4 for antigen+adjuvant test cohorts). An aqueous formulation for all adjuvants and antigens is ensured. Animals are immunized on Days 1, 15, and 28. A final test-bleed is performed via the marginal vein of the ear on Day 38. Sera may be banked at −80° C. for further analysis.

### Test Methods

### Inhibition of Cytopathic Effect (CPE) and Cell Death

A homogeneous assay was developed to quantify neutralization of ZIKV, and the consequent inhibition of cell death. The assay was adapted and standardized to 384-well plate formats, and the assay allows either near-real-time kinetic acquisition for six 384-well plates per experiment, or endpoint acquisition at the zenith of viral-induced cell death (3 days for ZIKV) for more than fifty 384-well plates.

Briefly, Vero cells were plated at a density of 10⁵ cells/mL in a 384-well plate (Plate 1). Separately, paired pre-immune/immune rabbit sera were serially diluted in 384-well plates in DMEM cell culture media containing propidium iodide (PI) at a concentration of 20 μg/mL (Plate 2). A stock of Zika virus was diluted so as to achieve a final multiplicity of infection (MOI) of 1, and added to the diluted pre-immune/immune sera wells. The plate was pre-incubated for 3 hours at 37° C. Following pre-incubation, samples from each well (Plate 1) containing serially diluted sera and virus were transferred onto the Vero cells (Plate 2). Cell death (indicated by PI staining of nuclei) was monitored in real-time using an IncuCyte imaging instrument (Essen BioScience, Inc., Ann Arbor, Mich.).

DENV-infected Vero cells also exhibit cytopathic effect leading to cell death, with the kinetics of CPE and cell death being not only time-dependent, but also a function of the multiplicity of infection and the strain of DENV. Analyses of the cell killing curves indicated that a significantly longer acquisition (up to 6 days) was necessary to capture DENV-induced CPE.

### Quantitation of Intracellular ZIKV E-Glycoprotein by Immunofluorescence

The pan-flavivirus 4G2 monoclonal antibody recognizes flavivirus group specific antigens (ZIKV, DENV, WNV, Japanese encephalitis virus (JEV), and YF) by binding to the fusion loop at the terminus of domain II of the E glycoprotein.¹³⁰⁻¹³² Intracellular E-glycoprotein content is a function of internalization (immediate post-infection), as well as replication.

An indirect immunofluorescence method was developed to quantify in vitro neutralization titers in antisera in Vero cells infected with ZIKV/DENV/YF17D. Vero cells are plated at a density of 10⁵ cells/mL in 384-well plates and infected at an MOI of 5 in the presence of serially diluted preimmune/immune rabbit sera. After an appropriate incubation period (for example, 3 days for ZIKV and YF17D, 4.5 days for DENV), cells are washed, permeabilized and fixed with 4% paraformaldehyde. A 1:500 dilution of 4G2, followed by anti-mouse IgG-AlexaFluor488 conjugate at a dilution of 1:200 (and PI for nuclear counterstain) is used for direct interrogation and quantitation of infected cells in a high-throughput manner (>20 plates/experiment).

Quantitation of ZIKV Copy Number Using qRT-PCR

Total RNA from 96 samples are extracted concurrently using an Aurum Total RNA 96 Kit (Bio-Rad, Hercules, Calif.). cDNA synthesis of the ZIKV strand is carried out with the reverse primer (5′-CTGTTCCACACCA CAAGCAT-3′ (SEQ ID NO: 20)) and an initial hybridization at 65° C.

Standard Superscript II Reverse Transcriptase/RNase H protocols. qPCR is performed on a CFX-96 Real Time System (Bio-Rad, Hercules, Calif.) with the following primers for ZIKV NS5: Forward primer: 5′-AGGCTGAGGAAGTGCTAGAG-3′ (SEQ ID NO: 21); Reverse primer: 5′-TGAGGGCATGTGCAAACCTA-3′ (SEQ ID NO: 22). The resultant amplicon length is 164 nucleotides with a melting temperature of 82.5° C. Detection limit: 5 copies/mL.

### Assessment of Neutralizing Antibodies Using GFP-Expressing Recombinant Viral Particles

Neutralizing antibodies were assessed using GFP-expressing recombinant viral particles (ZIKV, WNV, DENV-1, DENV-2, DENV-3, DENV-4); cellular infection was assayed using longitudinal intravital epifluorescence microscopy and automated image analysis on an INCUCYTE instrument (Essen BioScience, Inc., Ann Arbor, Mich.).

### Example 1

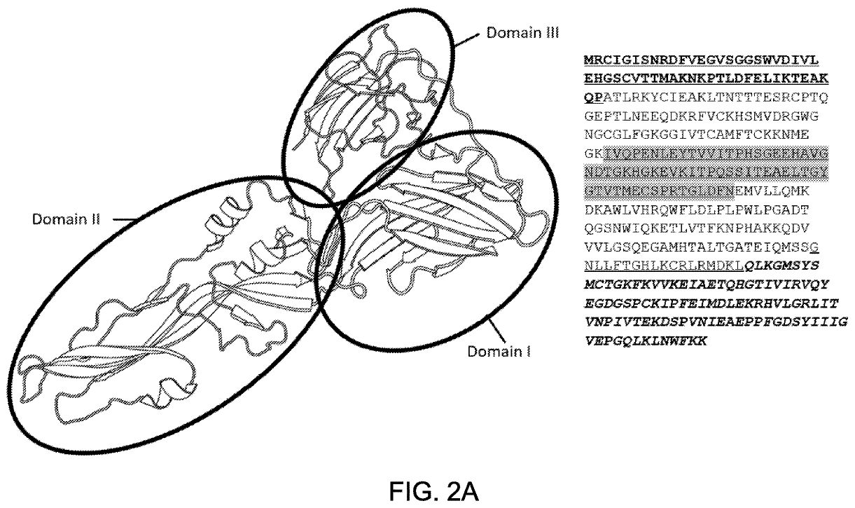

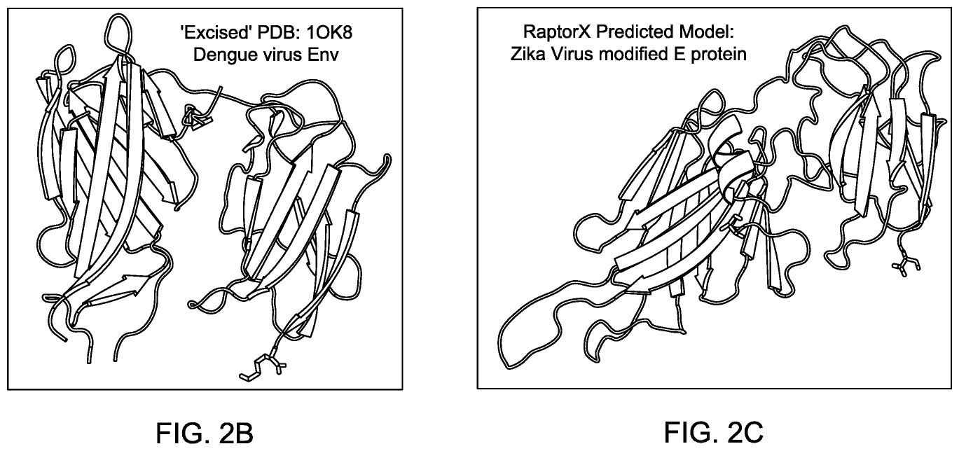

Because neutralizing antibodies against DENV and ZIKV may be directed to its surface-expressed E (ENV) glycoprotein, and given the high degree of sequence similarity between DENV and ZIKV, the crystal structure of DENV ENV protein (post-fusion conformation, soluble fragment, PDB coordinates: 1OK8) was used as a point of departure. Domain III (involved in receptor binding, and a major site for neutralizing antibodies) is discontinuous with Domain II (membrane fusion domain) and Domain I (FIG. 2).

In silico modeling of a re-engineered DENV-E having and amphipathic membrane fusion Domain II excised and the resultant discontinuous sequences ligated sequences with appropriate spacer peptides yielded a sequence which, on template-based protein structure modeling using RaptorX,¹⁰¹⁻¹⁰³ predicted a tertiary structure that faithfully reproduced the overall β-barrel fold topology of Domains I and III (FIG. 2). The homologous sequences in ZV-Env were identified by blastp, (see Table 1). The ZV-Env construct was expressed in E. coli as inclusion bodies, refolded, and purified.

The ZIKV antigen of Table 1 was fused to MBP and was expressed as described in the MATERIALS section. The antigen was tested using the Standardized Rabbit Model of Immunogenicity. Very high titers of anti-ZIKV antibodies were elicited in rabbits, but no neutralization was observed in vitro.

TABLE 1

| | | |
|---|---|---|
| DENV- | MRCIGISNRDFVEGVSGGSWVDIVLEHGSCVTTMAKNKPTLDFELIKTEA | SEQ |
| derived | KQPGIVQPENLEYTVVITPHSGEEHAVGNDTGKHGKEVKITPQSSITEAELT | ID |
| antigen | GYGTVTMECSPRTGLDFNGSSGNLLFTGHLKCRLRMDKLQLKGMSYSMCT | NO: |
| | GKFKVVKEIAETQHGTIVIRVQYEGDGSPCKIPFEIMDLEKRHVLGRLITVNP | 29 |
| | IVTEKDSPVNIEAEPPFGDSYIIIGVEPGQLKLNWFKK | |
| ZENV- | IRCIGVSNRDFVEGMSGGTWVDVVLEHGGCVTVMAQDKPTVDIELVTTTVSN | SEQ |
| derived | MGSIQPENLEYRIMLSVHGSQHSGMIVNDTGYETDENRAKVEVTPNSPRAEAT | ID |
| antigen | LGGFGSLGLDCEPRTGLDFSGGAKGKLFSGHLKCRLKMDKLRLKGVSYSLCTA | NO: |
| | AFTFTKVPAETLHGTVTVEVQYAGTDGPCKIPVQMAVDMQTLTPVGRLITANP | 30 |
| | VITESTENSKMMLELDPPFGDSYIVIGVGDKKITHHWHRS | |

### Example 2

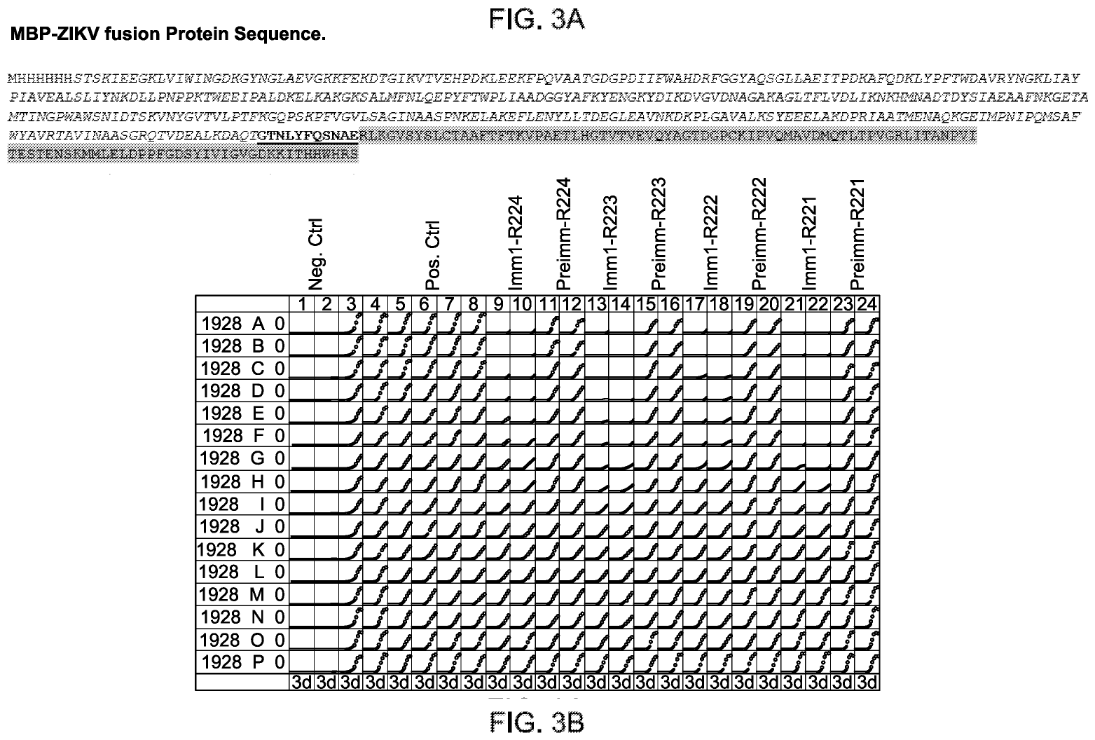

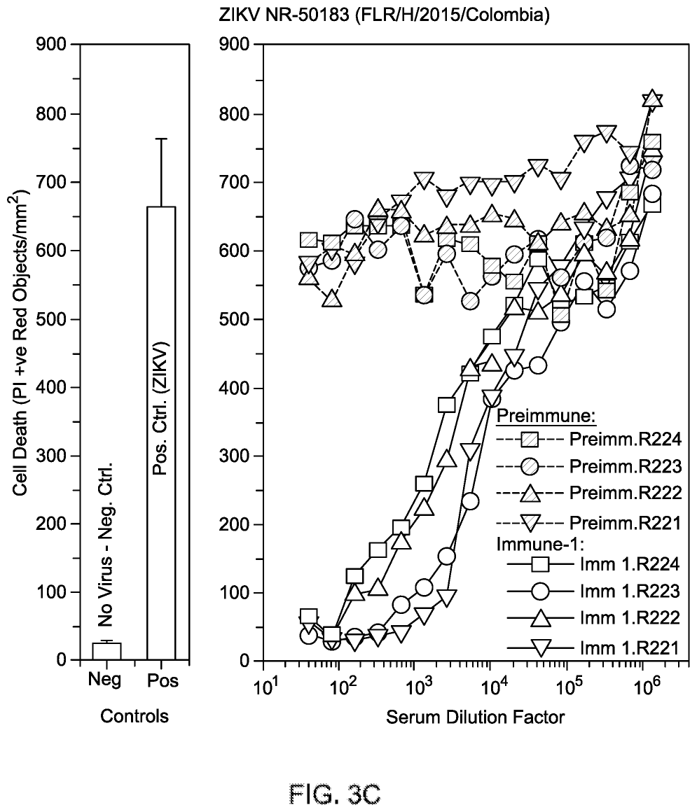

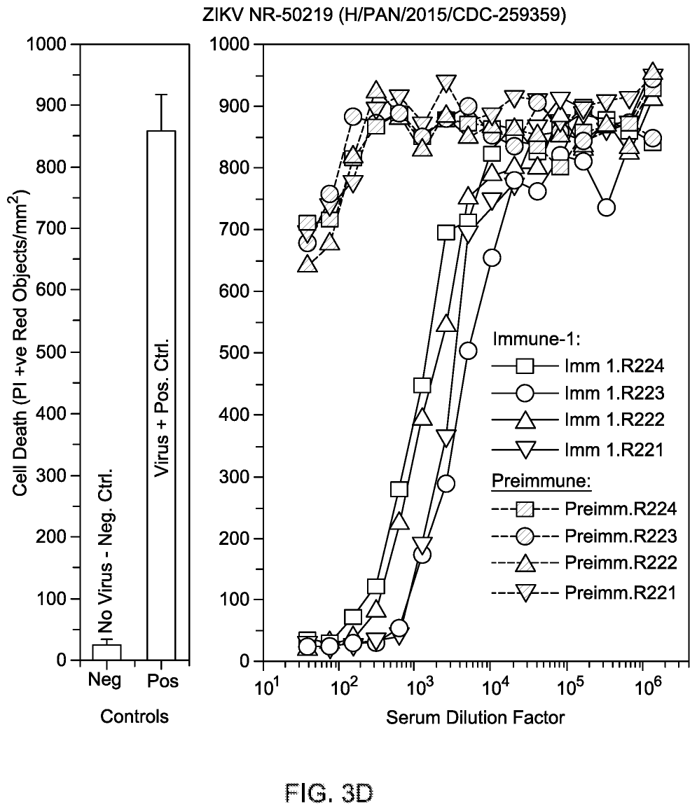

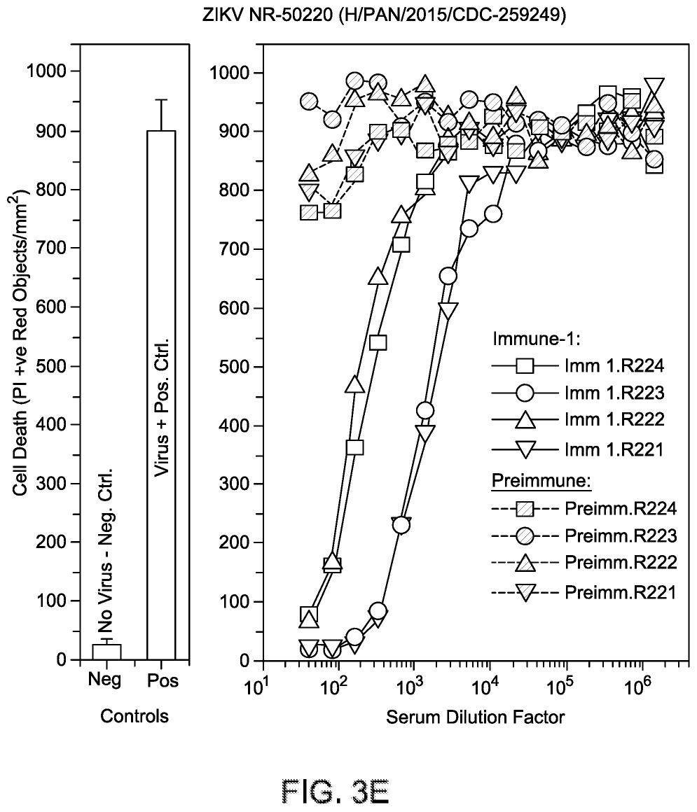

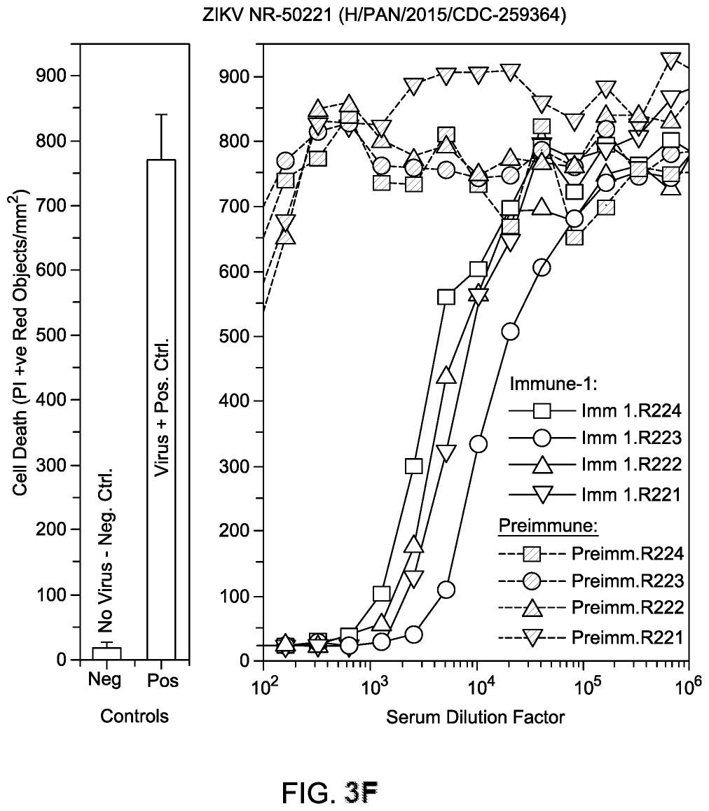

Because no neutralization was observed using the antigen of Example 1, several modifications of the ZIKV sequence used in Example 1 were tested. After several iterations, sera from rabbits immunized with 10 μg/dose of a maltose binding protein (MBP)-ZIKV E-glycoprotein fusion construct having the sequence shown in Table 2 (which is much shorter than the fusion construct of Example 1) was obtained, and ZIKV-neutralizing activity was observed in the sera (FIG. 3).

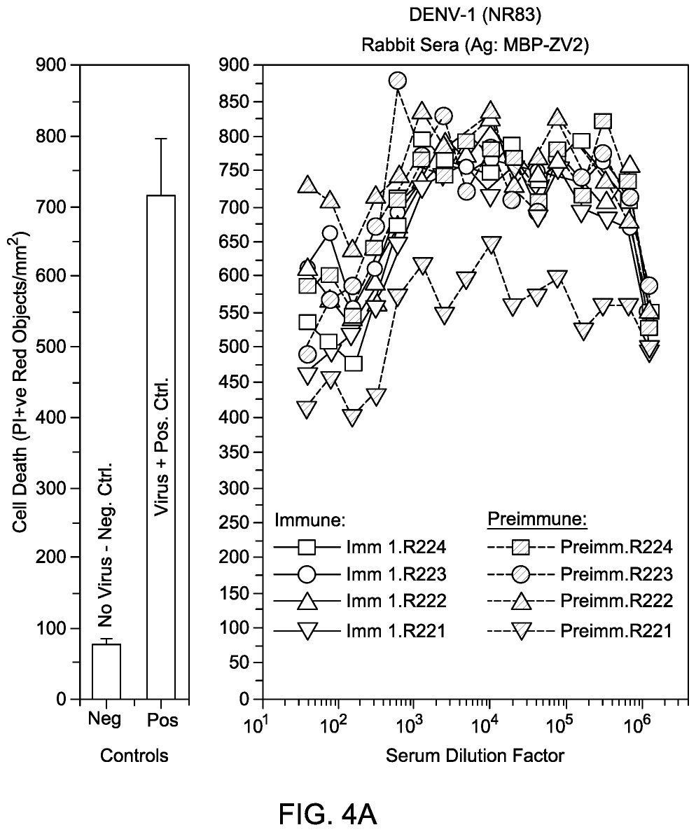

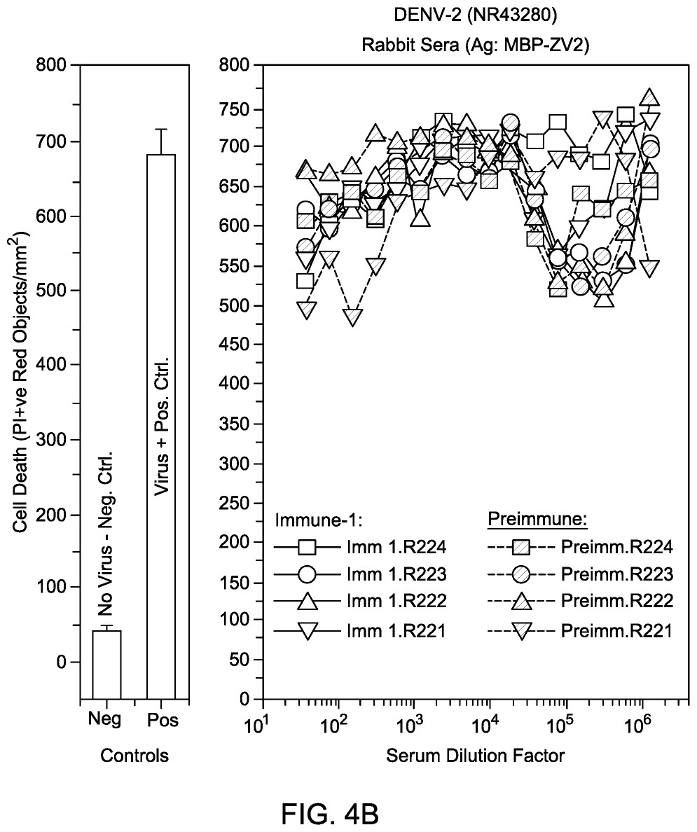

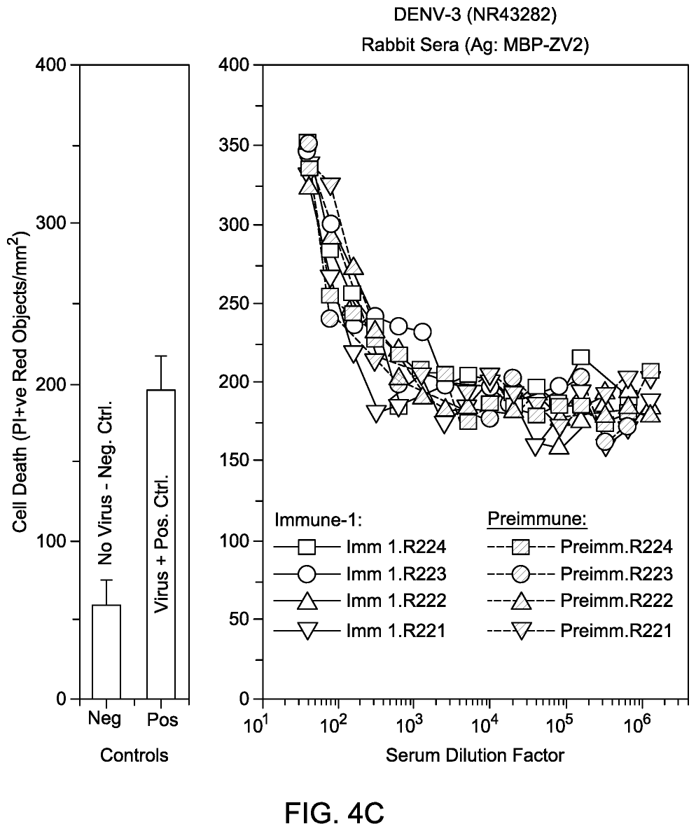

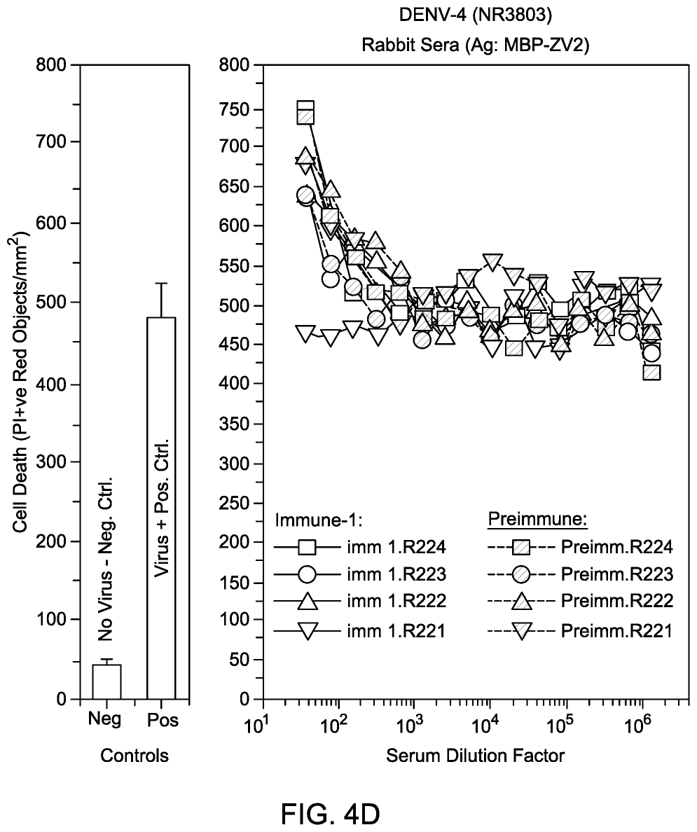

Because of concerns of the Original Antigenic Sin⁵⁹, and Antibody Dependent Enhancement (ADE) documented in flaviviral infections⁶⁷⁻⁷³, whether antisera raised against MBP-ZIKV would exhibit ADE (or heterologous protection) against DENV-1, DENV-2, DENV-3, DENV-4 or YF17F was examined. Neither ADE, nor heterologous protection was observed. FIG. 4 shows the absence of antibody-dependent enhancement (ADE) or heterologous protection against DENV-1, DENV-2, DENV-3, or DENV-4 immune sera from rabbits immunized with MBP-ZIKV using the CPE assay. Essentially identical results were obtained with both PI/cell death and 4G2 immunostain methods. No ADE/heterologous protection was observed with YF17D, as well.

TABLE 2

| | | |
|---|---|---|
| MBP- | MHHHHHHSTSKIEEGKLVIWINGDKGYNGLAEVGKKFEKDTGIKVTVEHPD | SEQ ID |
| ZIKV | KLEEKFPQVAATGDGPDIIFWAHDRFGGYAQSGLLAEITPDKAFQDKLYPFT | NO: 31 |
| | WDAVRYNGKLIAYPIAVEALSLIYNKDLLPNPPKTWEEIPALDKELKAKGKS | |
| | ALMFNLQEPYFTWPLIAADGGYAFKYENGKYDIKDVGVDNAGAKAGLTFLV | |
| | DLIKNKHMNADTDYSIAEAAFNKGETAMTINGPWAWSNIDTSKVNYGVTVLP | |
| | TFKGQPSKPFVGVLSAGINAASPNKELAKEFLENYLLTDEGLEAVNKDKPLGA | |
| | VALKSYEEELAKDPRIAATMENAQKGEIMPNIPQMSAFWYAVRTAVINAASG | |
| | RQTVDEALKDAQTGTENLYFQSNARLKGVSYSLCTAAFTFTKVPAETLHGT | |
| | VTVEVQYAGTDGPCKIPVQMAVDMQTLTPVGRLITANPVITESTENSKMM | |
| | LELDPPFGDSYIVIGVGDKKITHHWHRS | |
| ZIKV | SNARLKGVSYSLCTAAFTFTKVPAETLHGTVTVEVQYAGTDGPCKIPVQM | SEQ ID |
| | AVDMQTLTPVGRLITANPVITESTENSKMMLELDPPFGDSYIVIGVGDKKIT | NO: 1 |
| | HHWHRS | |

### Example 3

### Immunodominance of the MBP Fragment

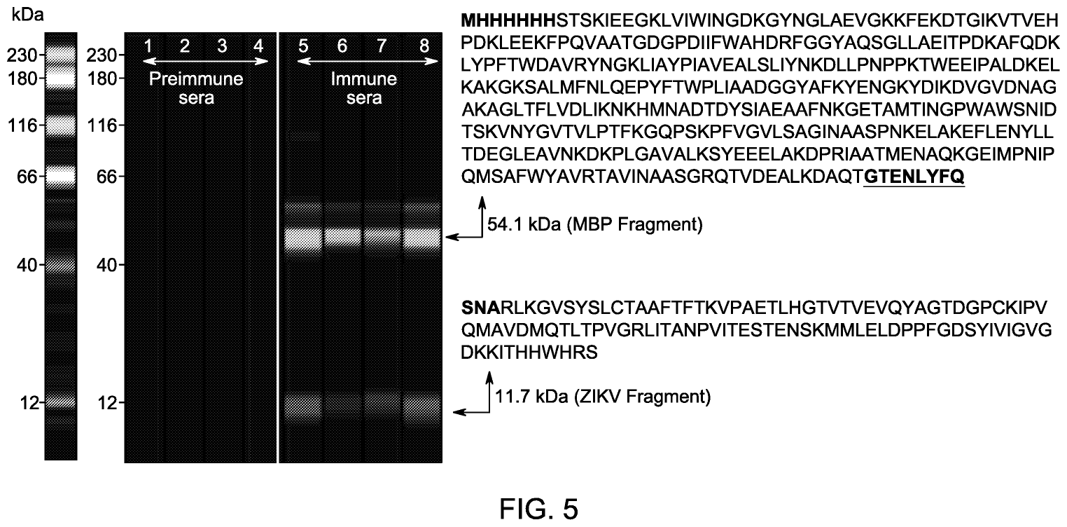

A comparison of the relative mases of the His-tag/MBP fragment (376 residues, 41,437.9 Daltons) versus the ZIKV fragment (108 residues, 11,717.4 Daltons) indicated that the MBP fragment, while allowing correct folding of the protein and greatly enhancing yields of soluble protein, was quite large and could be immunodominant. The Tobacco Etch Virus (TEV) protease^{136, 137} cut site that was engineered in the protein (see FIG. 3) allowed testing of whether the MBP fragment was immunodominant. The MBP-ZIKV fusion protein was cleaved with TEV protease and probed with the immune sera using Western blots (FIG. 5). As evidenced by the enhanced recognition of the MBP fragment compared to the ZIKV fragment, immunodominance of the MBP fragment was observed.

### Large-Scale Purification of the ZIKV Fragment

To isolate the ZIKV fragment from the fusion protein, a large-scale batch of the fusion protein was expressed, purified, and cleaved using TEV protease (also having a His-tag). The cleavage reaction was performed at 4° C. overnight. Before re-loading on the Ni²⁺⁻NTA resin (approximate 4 mg of cleaved fusion protein/mL resin) to remove the MBP fragment and the TEV (both have an N-terminal His-tag), the sample was dialyzed against the dialysis buffer (PBS plus 88 mM mannitol) for 4 hours to remove imidazole. Re-loading was performed at 4° C. and flow-through fractions containing the ZIKV fragment was collected and characterized in detail using a range of techniques not only to assess purity, but also to verify disulfide formation, monodisperisty, and conformational homogeneity as described below. ¹⁵N-labeled ZIKV fragment was also isolated for ¹⁵N/¹H heteronuclear single quantum coherence spectroscopy ¹³⁸ (HSQC) NMR experiments.

### Characterization of the ZIKV Fragment

Size exclusion chromatography showed a monomeric species eluting at a volume corresponding to 12 kDa, and LC-MS on an accurate mass QTOF instrument showed the expected mass of 11,717.95 Daltons, indicating that the two cysteines were oxidized (intramolecular disulfide). A melting temperature of 54.2° C. in thermal shift assays with SYPRO Orange^{139, 140}, indicated a stable, well-folded structure, which was confirmed by ¹⁵N-¹H heteronuclear single quantum coherence (HSQC) NMR experiments.

This fragment, in conjunction with EY-4-143 as an adjuvant, was evaluated using the standardized rabbit immunogenicity modified as follows: an antigen concentration of 50 μg/dose was used.

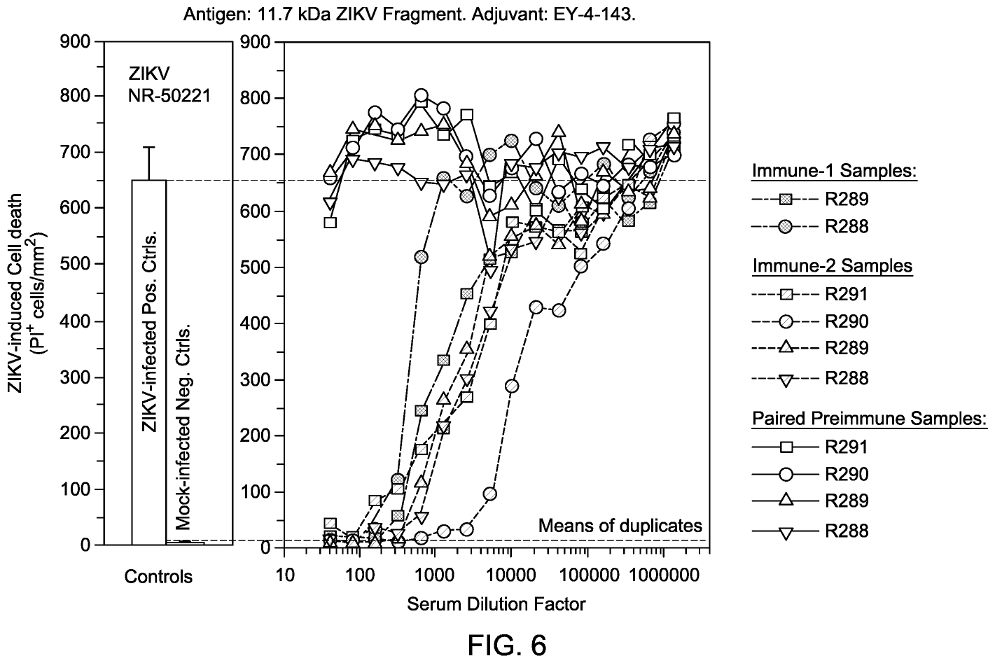

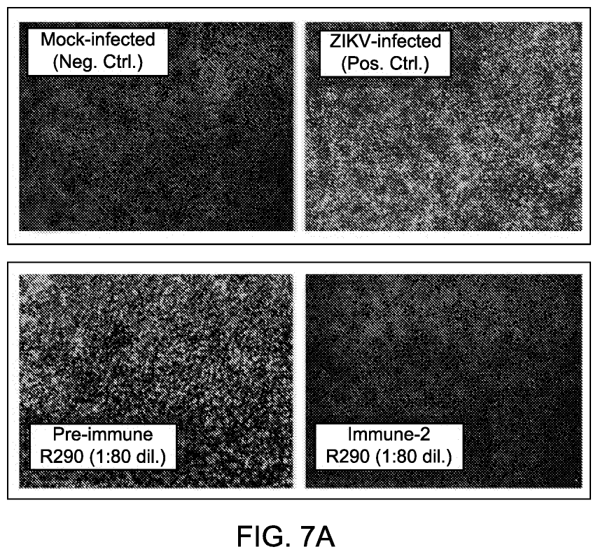

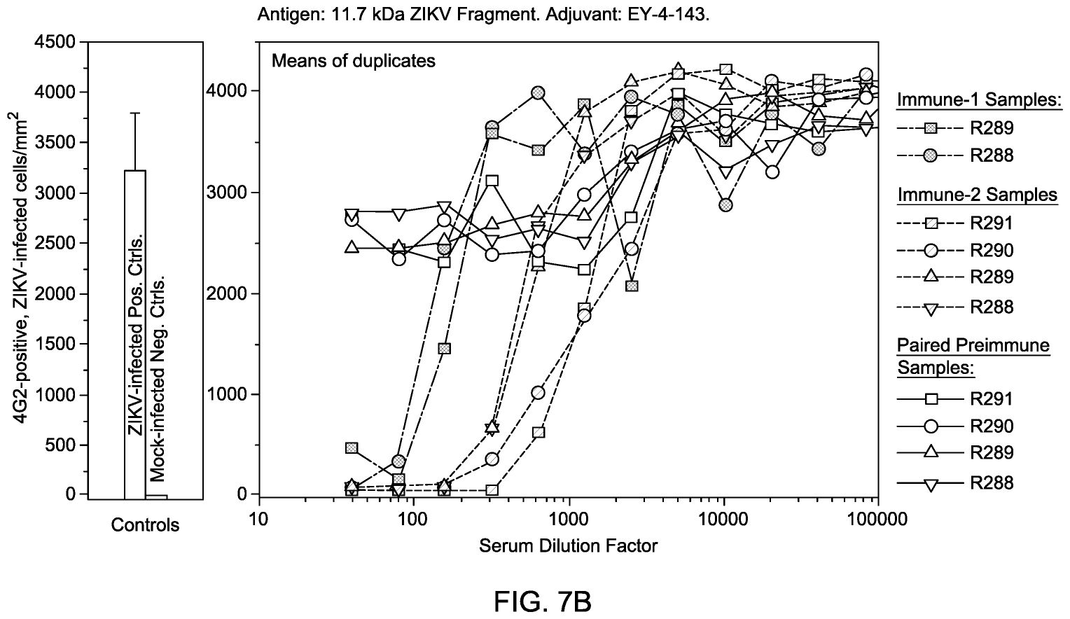

Immune-1 sera (obtained a week following the 1^{st} boost), Immune-2 sera (obtained after the 2^{nd} boost), as well as pre-immune sera were examined for anti-ZIKV IgG in standard ELISAs. In vitro neutralization of ZIKV was quantified using both the CPE/cell-death assay and 4G2 immunostaining which interrogates intracellular ZIKV E-glycoprotein content. When adjuvanted with EY-4-143, the ZIKV fragment at 50 mg/dose consistently elicited high neutralizing titers in all animals (n=4), even after a single boost. All six strains of ZIKV tested (Table 3) were neutralized. Representative data showing one of six plates from the CPE/cell death assay and 4G2 immunostain assay, obtained with ZIKV NR50221 (H/PAN/2015/CDC-259364), are shown in FIG. 6 and FIG. 7, respectively.

TABLE 3

| Strain of ZIKV | Source |
|---|---|
| ZIKV/NR-50183: FLR/H/2015/Colombia | BEI Resources |
| ZIKV/NR-50219: H/PAN/2015/CDC-259359 | BEI Resources |
| ZIKV/NR-50220: H/PAN/2015/CDC-259249 | BEI Resources |
| ZIKV/NR-50221: H/PAN/2015/CDC-259364 | BEI Resources |
| ZIKV/NR-50240: PRVABC59 H/2015/Puerto Rico | BEI Resources |
| ZIKV/Thai: Accession # KF998678 | Dr. James Whitney, Harvard Medical School |

### Example 4

This Example describes the immunogenicity, protective efficacy, and cross reactivity (in vitro) of DENV-1, DENV-2, DENV-3, DENV-4, and WNV antigens developed using the principles of antigen design described above for ZIKV.

### Expression and Characterization of DENV-1, DENV-2, DENV-3, DENV-4 and WNV Immunogens

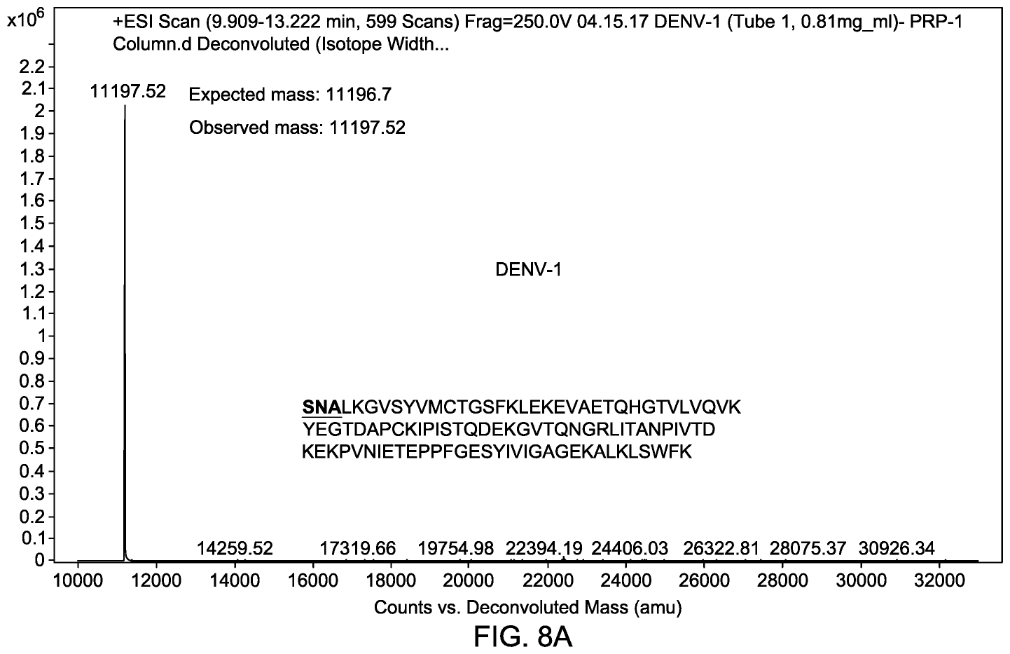

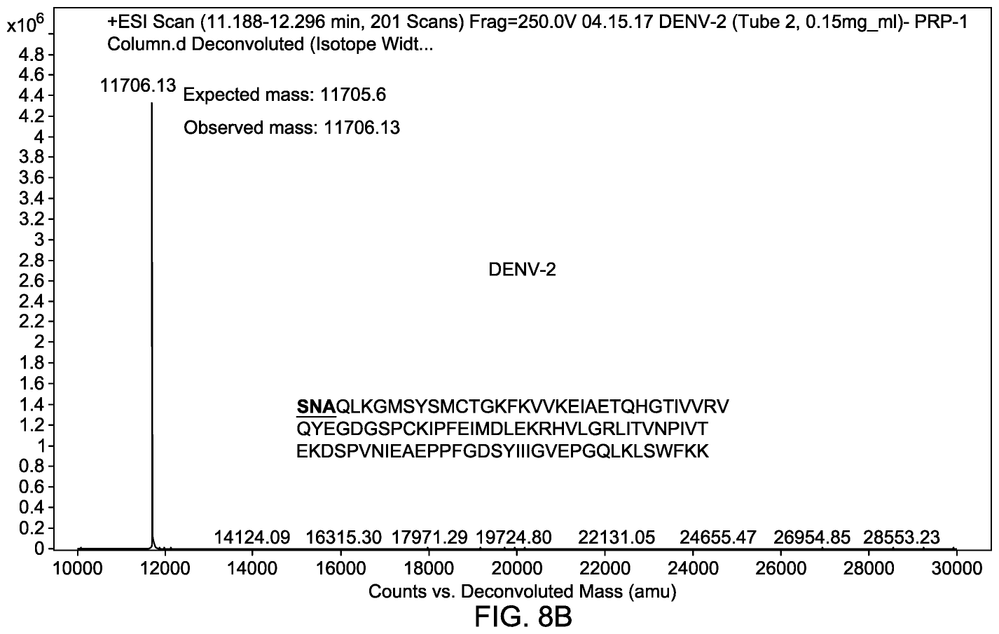

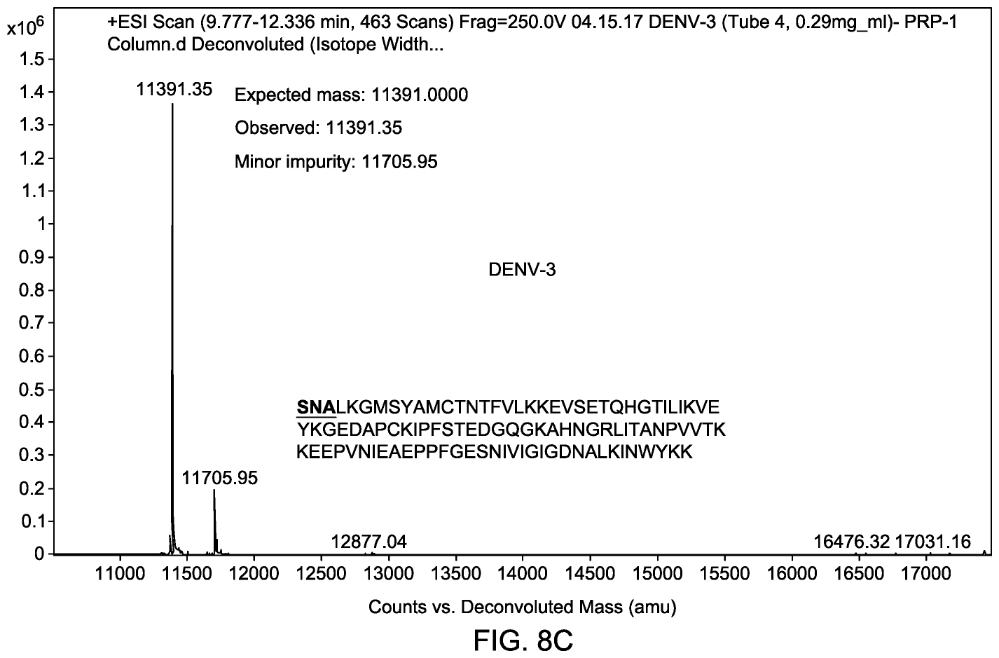

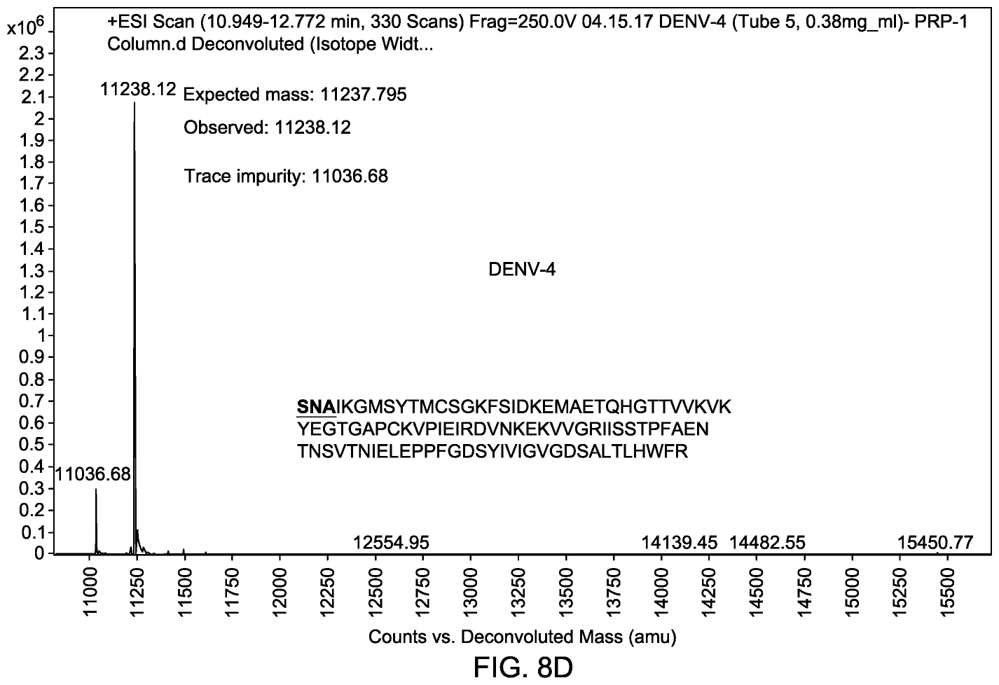

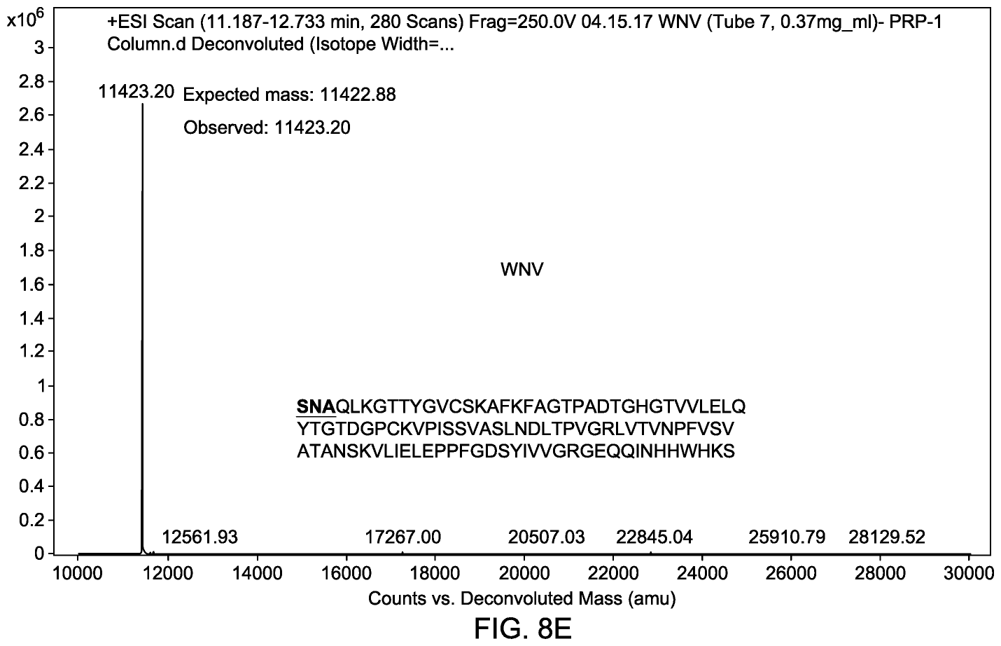

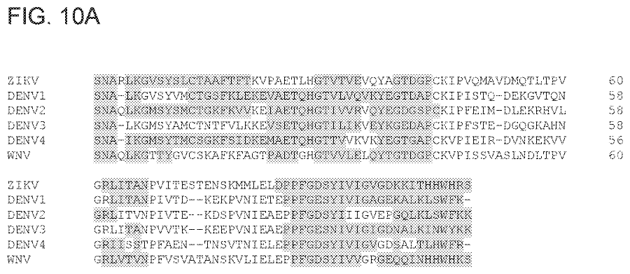

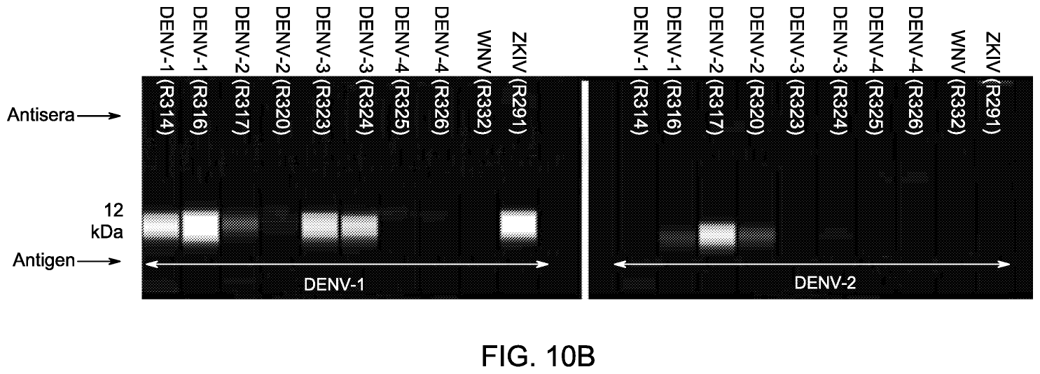

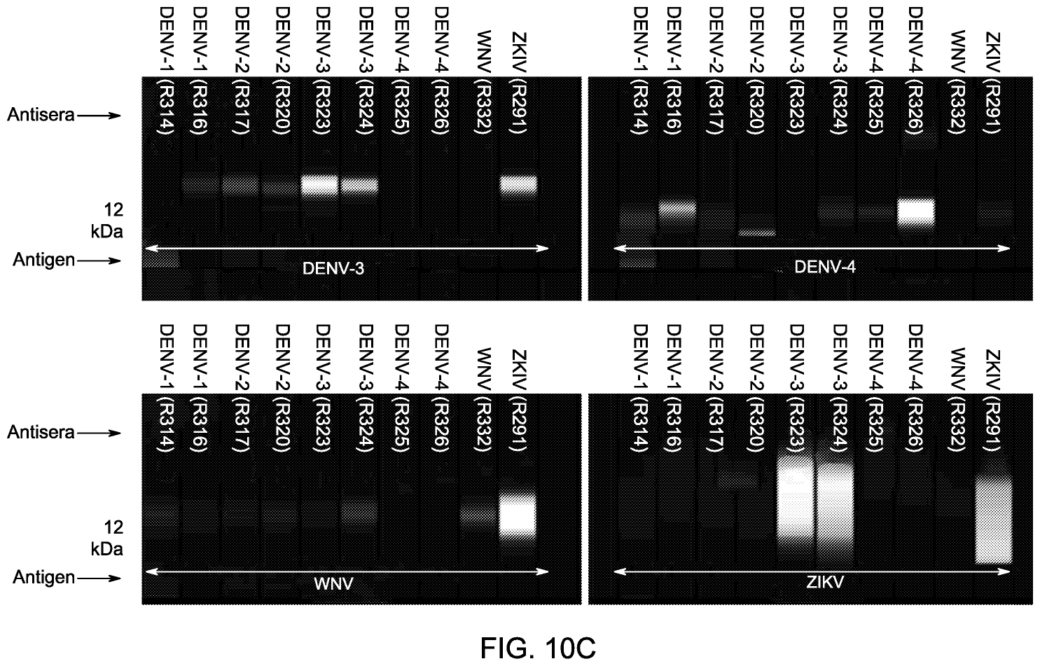

Homology mapping of the ZIKV sequence to DENV-1, DENV-2, DENV-3, DENV-4 and WNV E-glycoprotein sequences yielded target sequences (see Table 4). An alignment of these sequences is provided in FIG. 10A. The sequences were cloned (as fusion proteins), expressed, purified, and cleaved by TEV protease to obtain the desired antigens (FIG. 8).

TABLE 4

| | | SEQ | % identical | % identical |
|---|---|---|---|---|
| | | ID | residues vs | resides vs |
| Flavivirus | Antigen Sequence | NO: | ZIKV | DENV-1 |
| ZIKV | SNARLKGVSYSLCTAAFTFTKVPAETLHGTVTV | 1 | 100 | |
| | EVQYAGTDGPCKIPVQMAVDMQTLTPVGRLITANP | | | |
| | VITESTENSKMMLELDPPFGDSYIVIGVGDKKIT | | | |
| | HHWHRS | | | |
| DENV-1 | SNALKGVSYVMCTGSFKLEKEVAETQHGTVLV | 2 | 52 | 100 |
| | QVKYEGTDAPCKIPISTQDEKGVTQNGRLITANP | | | |
| | IVTDKEKPVNIETEPPFGESYIVIGAGEKALKLSWFK | | | |
| DENV-2 | SNAQLKGMSYSMCTGKFKVVKEIAETQHGTIVV | 3 | 49 | 64 |
| | RVQYEGDGSPCKIPFEIMDLEKRHVLGRLITVNP | | | |
| | IVTEKDSPVNIEAEPPFGDSYIIIGVEPGQLKLSWFKK | | | |
| DENV-3 | SNALKGMSYAMCTNTFVLKKEVSETQHGTILIKV | 4 | 49 | 70 |
| | EYKGEDAPCKIPFSTEDGQGKAHNGRLITANP | | | |
| | VVTKKEEPVNIEAEPPFGESNIVIGIGDNALKINWYKK | | | |
| DENV-4 | SNAIKGMSYTMCSGKFSIDKEMAETQHGTTVVKV | 5 | 51 | 56 |
| | KYEGTGAPCKVPIEIRDVNKEKVVGRIISSTPFAENT | | | |
| | NSVTNIELEPPFGDSYIVIGVGDSALTLHWFR | | | |
| WNV | SNAQLKGTTYGVCSKAFKFAGTPADTGHGTVVLE | 6 | 57 | |
| | LQYTGTDGPCKVPISSVASLNDLTPVGRLVTVNP | | | |
| | FVSVATANSKVLIELEPPFGDSYIVVGRGEQQINHHWHKS | | | |

### Homotypic Neutralizing Titers (DENV Strains)

Rabbits (n=4/cohort) were immunized with 10 μg DENV-1, DENV-2, DENV-3, DENV-4, or WNV antigens, as shown in Table 4, each adjuvanted with EY-4-143 (100 μg), as described in the Standardized Rabbit Model of Immunogenicity, to determine whether the antigens successfully induce neutralizing antibodies to the cognate strain (that is, homotypic protection in vitro).

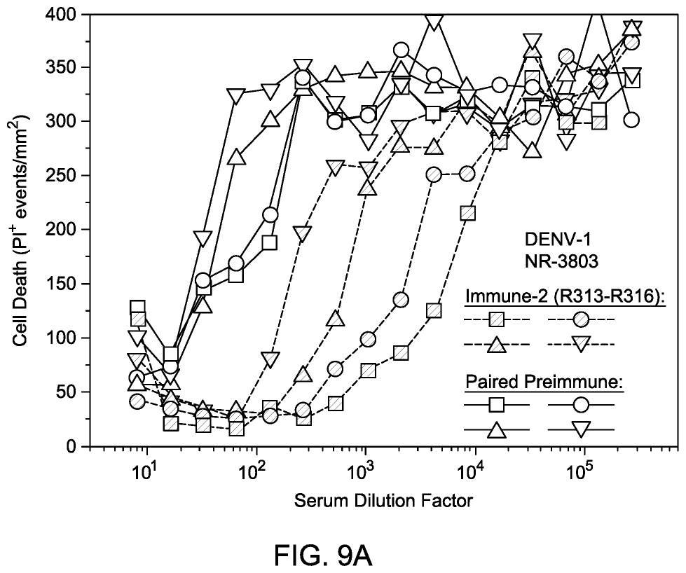

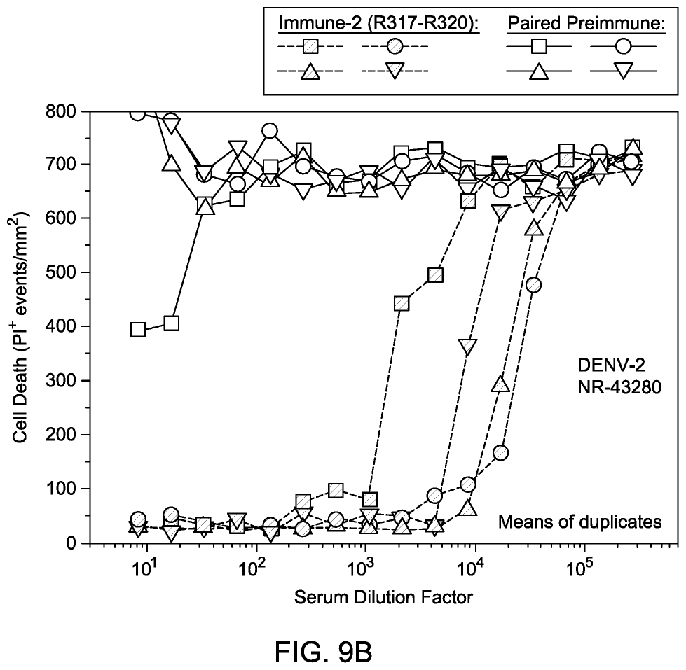

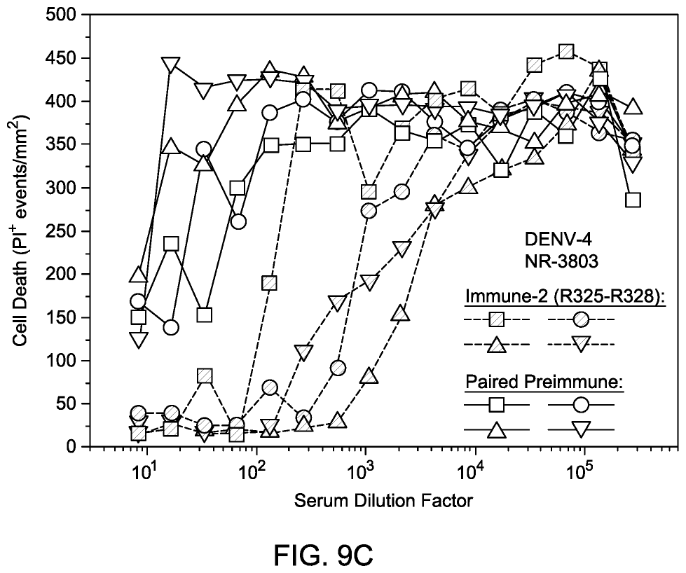

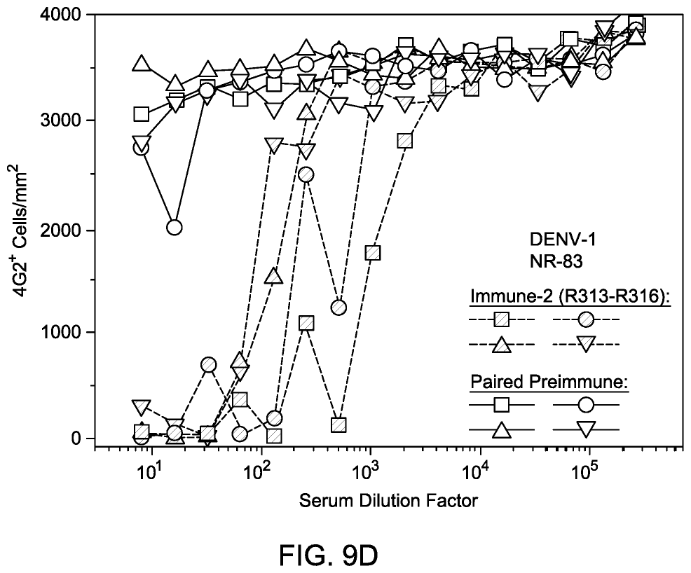

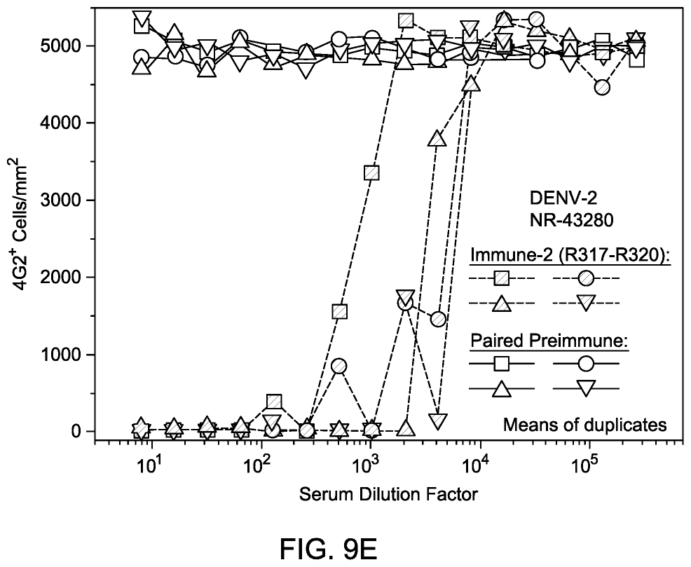

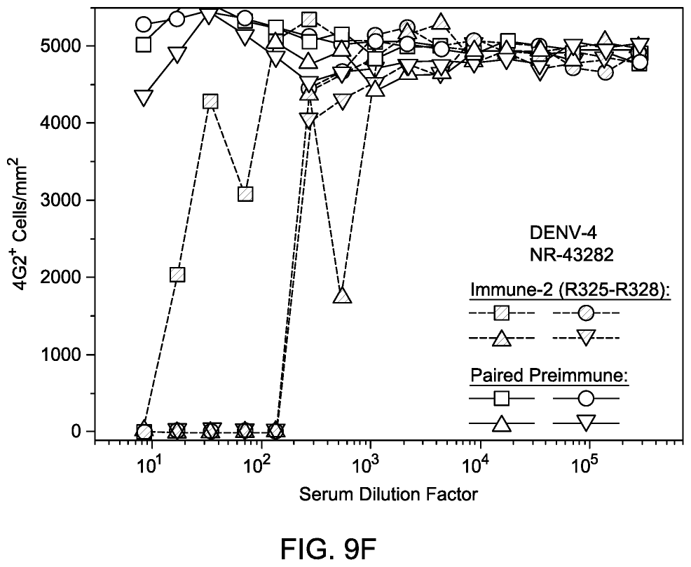

All animals with the exception of the cohort immunized with DENV-3 showed in vitro neutralization. (FIG. 9).

Additional rabbits will be immunized with 50 μg DENV-3 antigen, adjuvanted with EY-4-143, as described in the Standardized Rabbit Model of Immunogenicity. The increased dose of the antigen is expected to successfully induce neutralizing antibodies to the cognate strain.

### Heterologous Neutralization in DENV and WNV Antisera

A high degree of sequence similarity is observed even in the approximately 12 kDa C-terminal domain between ZIKV, the DENV strains, and WNV (FIG. 10A), and serological cross reactivity was detected. Significant cross-reactivity between DENV-3 antisera and DENV-1 antigen was noted; reciprocal cross-reactivity was evident between ZIKV and DENV-3; there was also strong cross-reactivity between ZIKV antisera and WNV antigen (FIG. 10B-FIG. 10C).

To examine the consequences of such cross-reactivity, noting that it could range from ADE to cross-protection (or no effect at all), the effect of sera from rabbits immunized with each antigen on viral replication of the other antigens was examined.

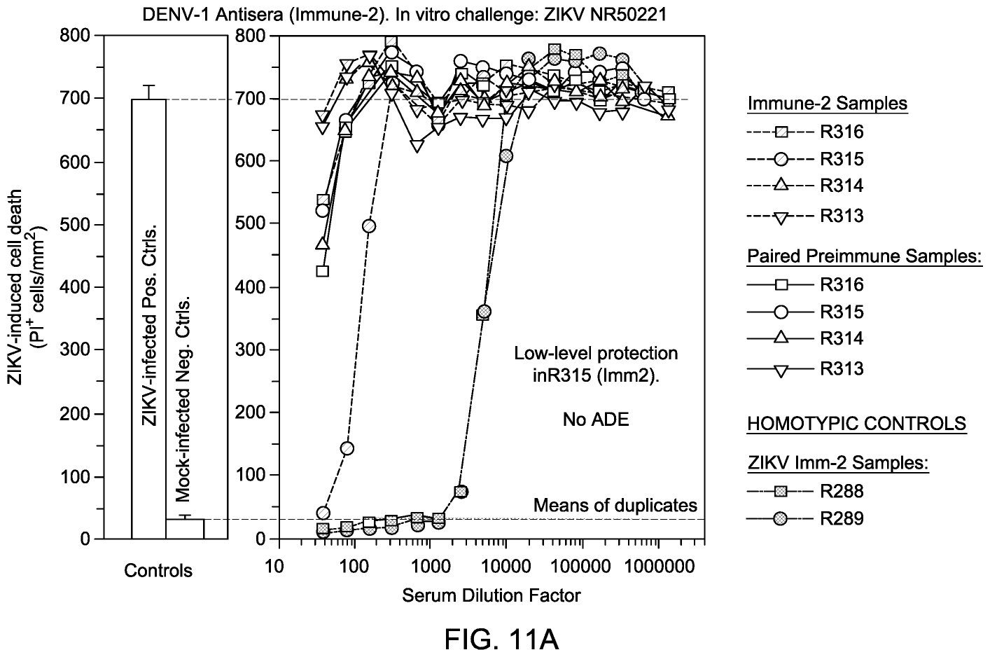

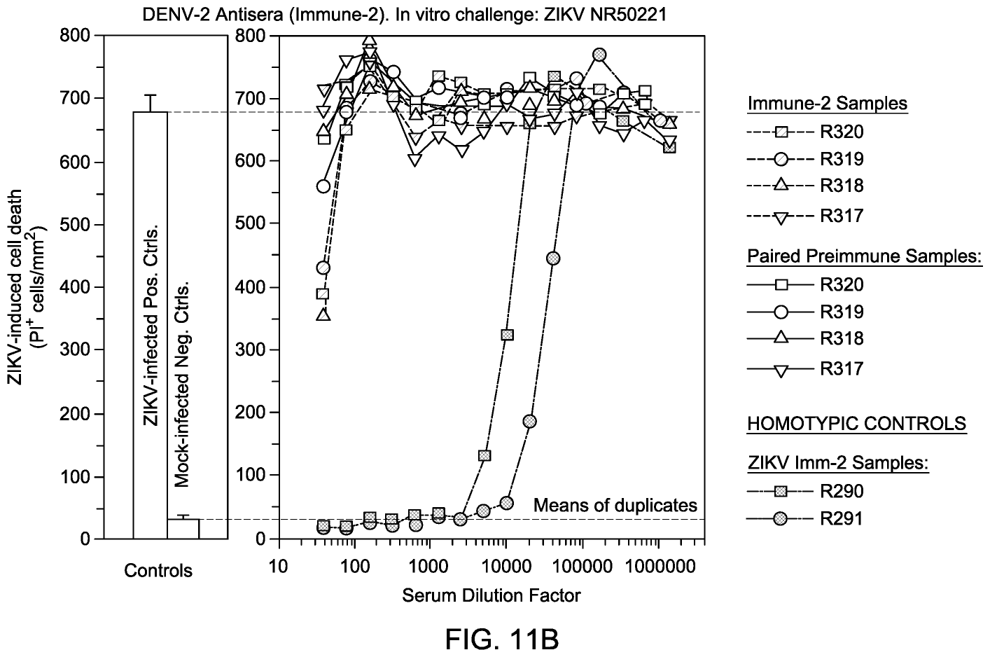

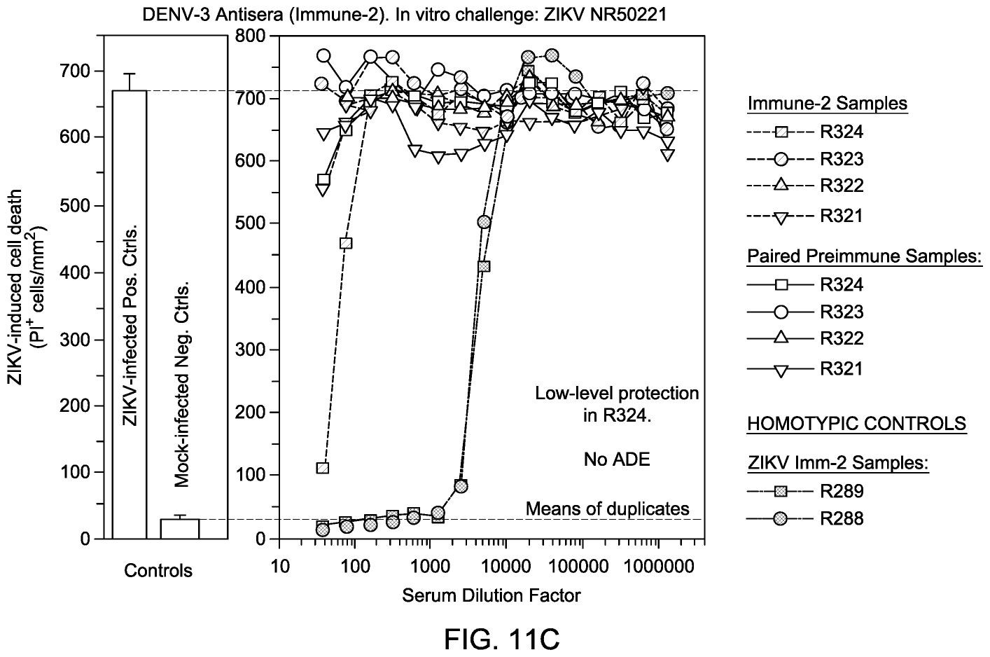

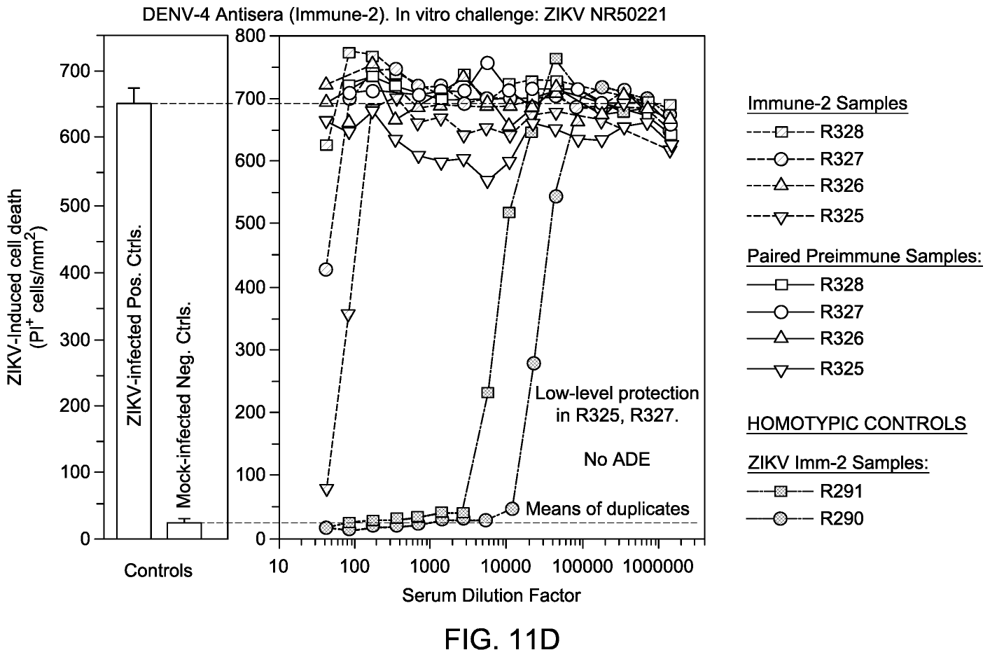

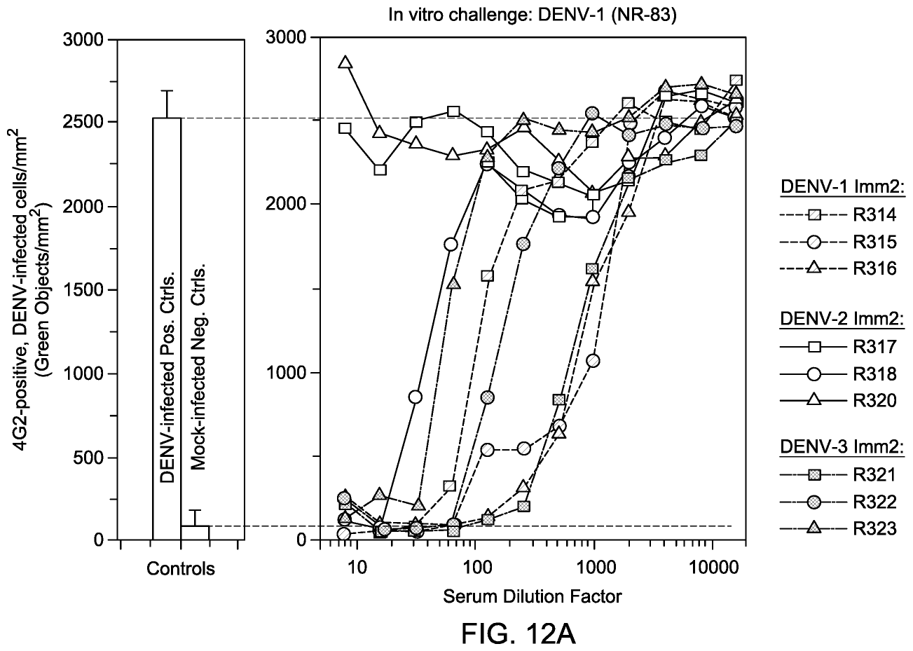

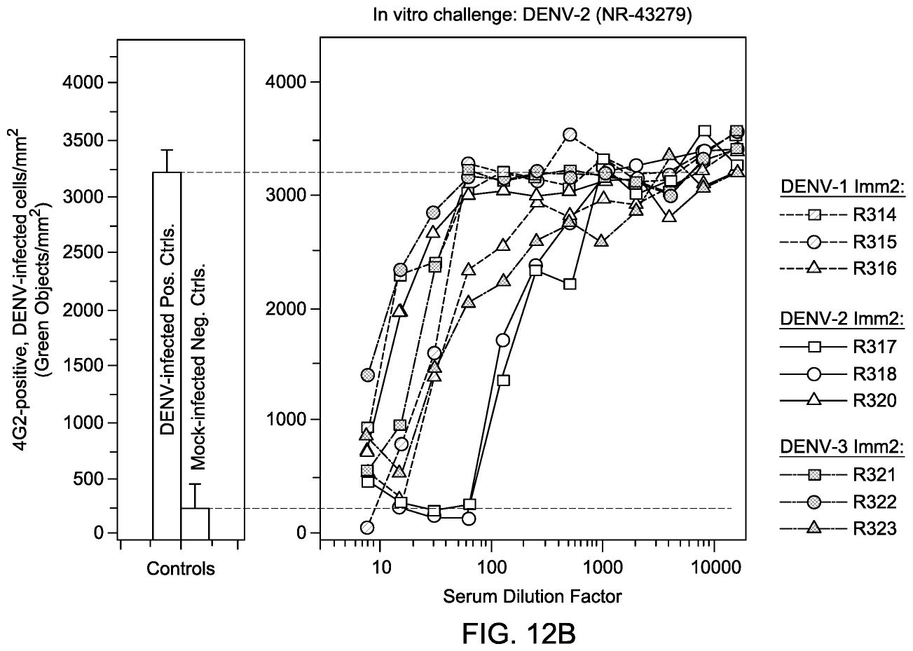

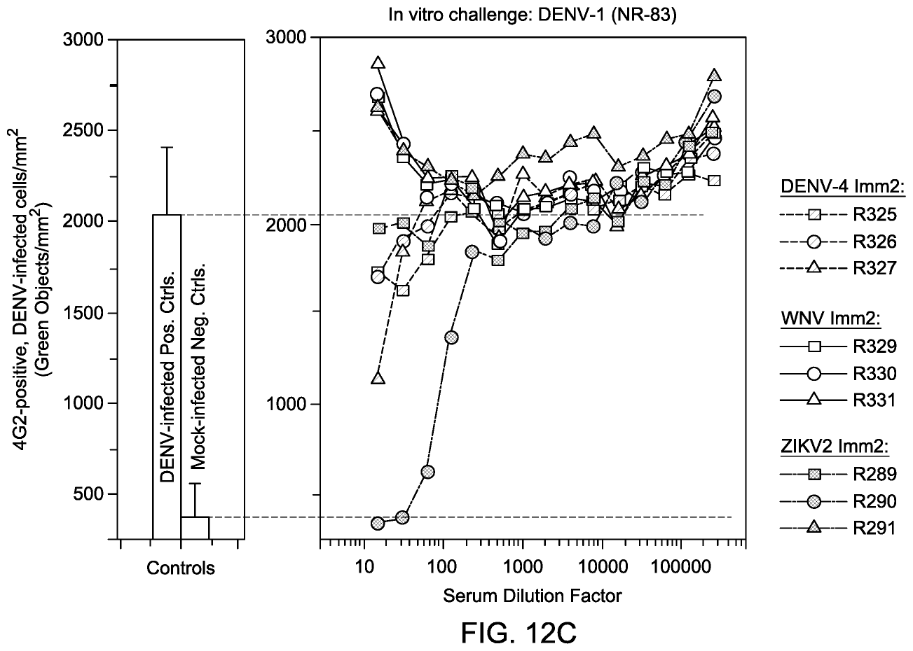

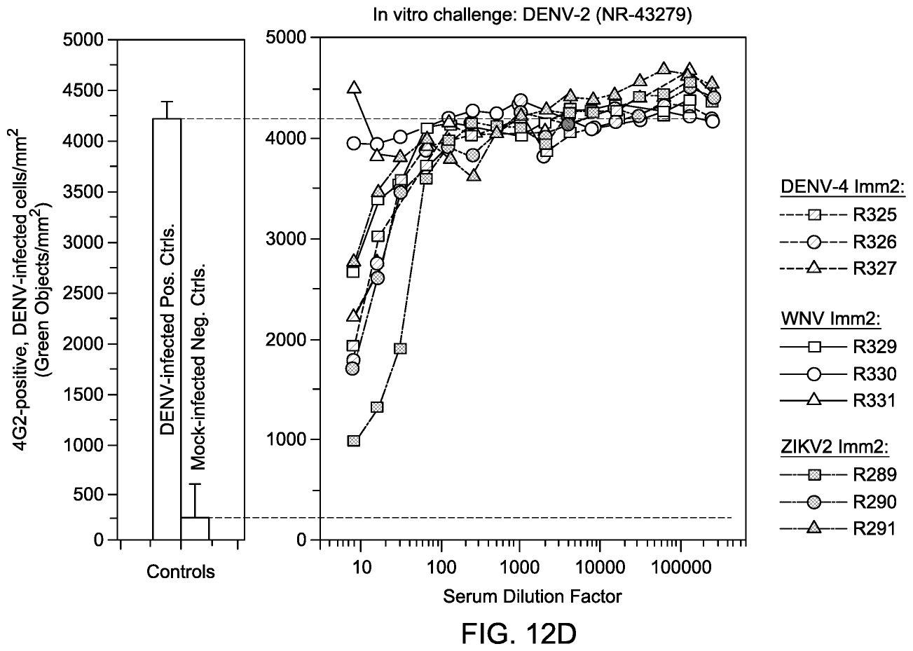

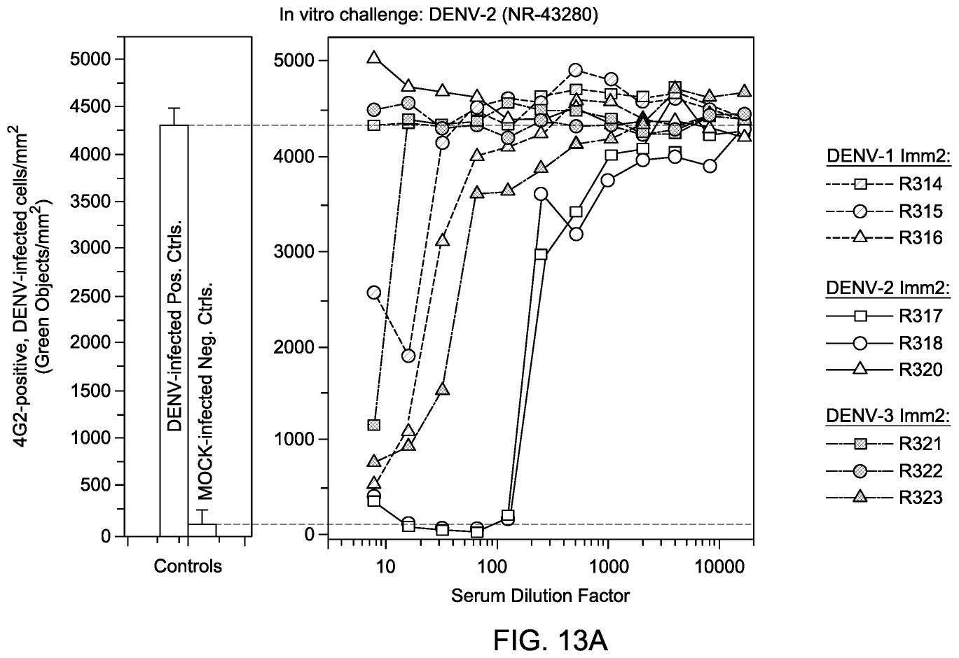

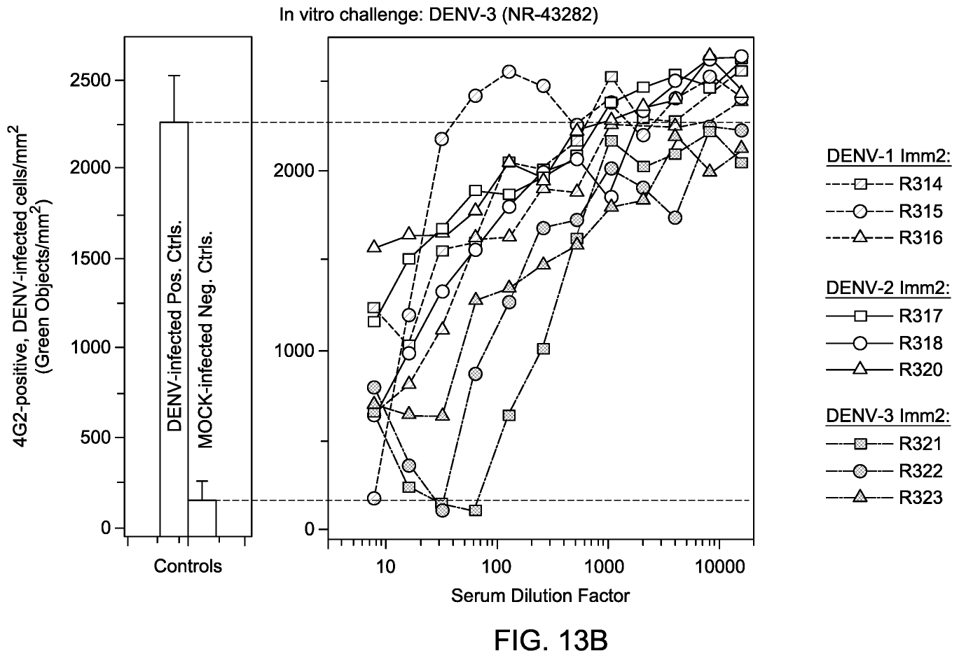

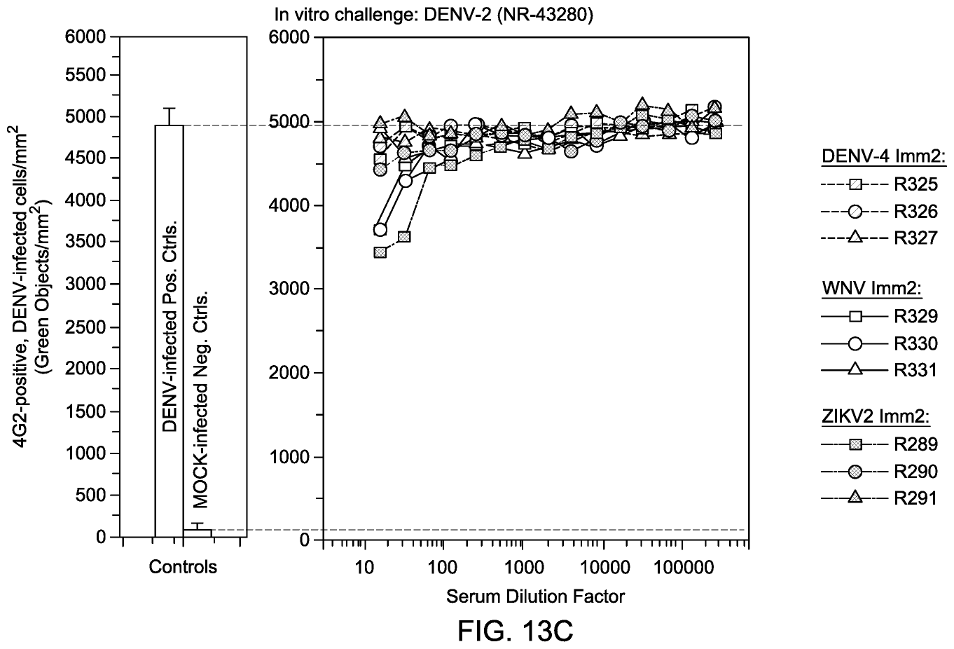

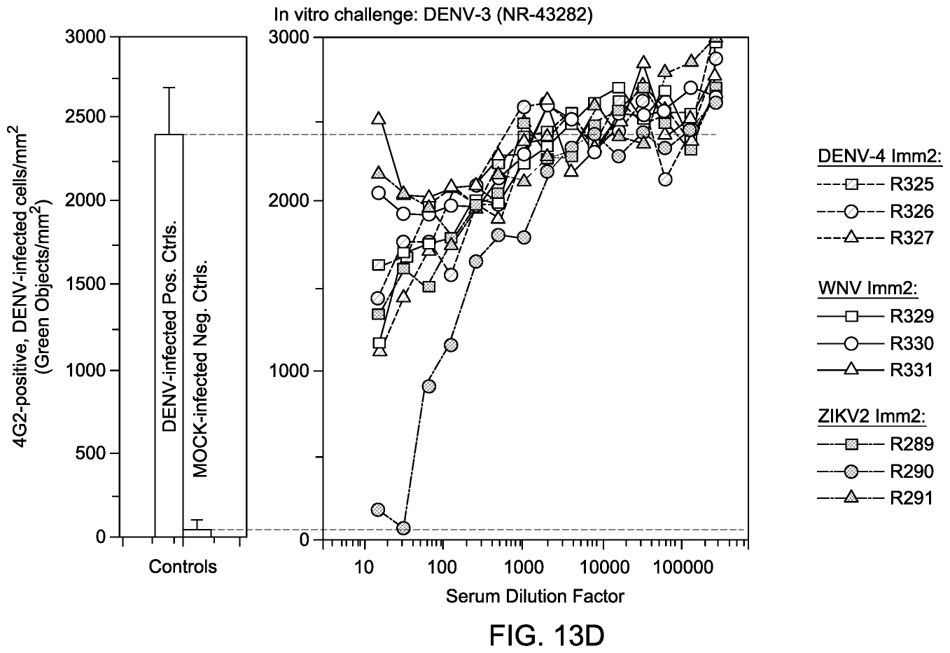

Sera from rabbits immunized with DENV-1, DENV-2, DENV-3, DENV-4 antigens exhibited sporadic low level inhibition of ZIKV replication (FIG. 11), and no enhancement was observed, ruling out ADE (for ZIKV). Antisera to DENV-3 neutralizes DENV-1 (FIG. 12), and DENV-4 (FIG. 13). In cases where some values appeared to above controls (FIG. 13C), no differences were detected between pre-immune and immune samples, and ADE was therefore ruled out.

### Example 5

This Example shows the Interaction of EY-4-143 with ZIKV, DENV-1, DENV-2, DENV-3 and DENV-4 antigens.

The calculated isoelectric point (pI) values for the ZIKV, DENV-1, DENV-2, DENV-3 and WNV antigens ranged between 6.2 and 6.4 and, consequently, significant Coloumbic interactions between the highly polyanionic hyaluronic acid backbone of EY-4-143 and the subunit immunogens were not expected.

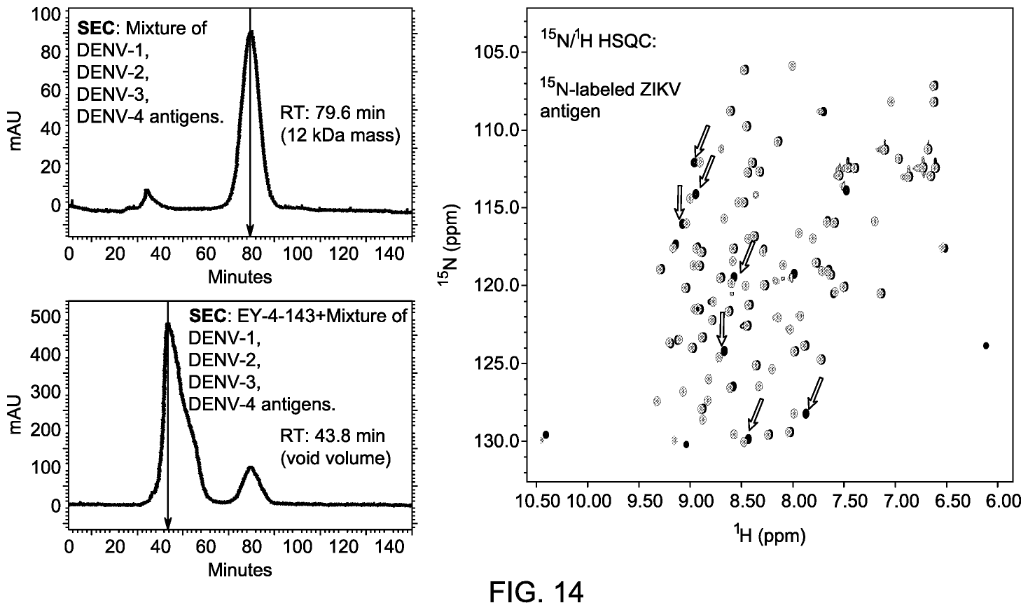

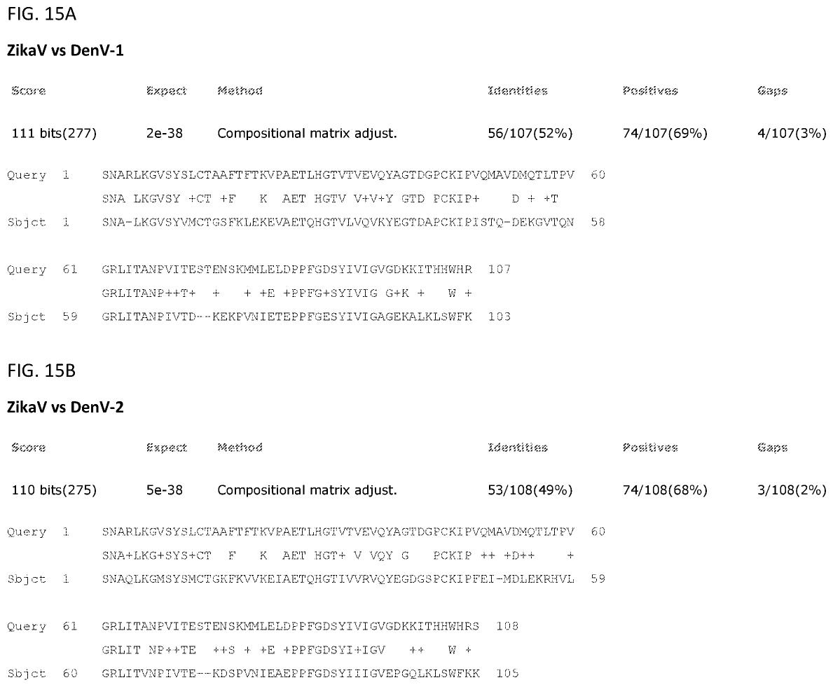

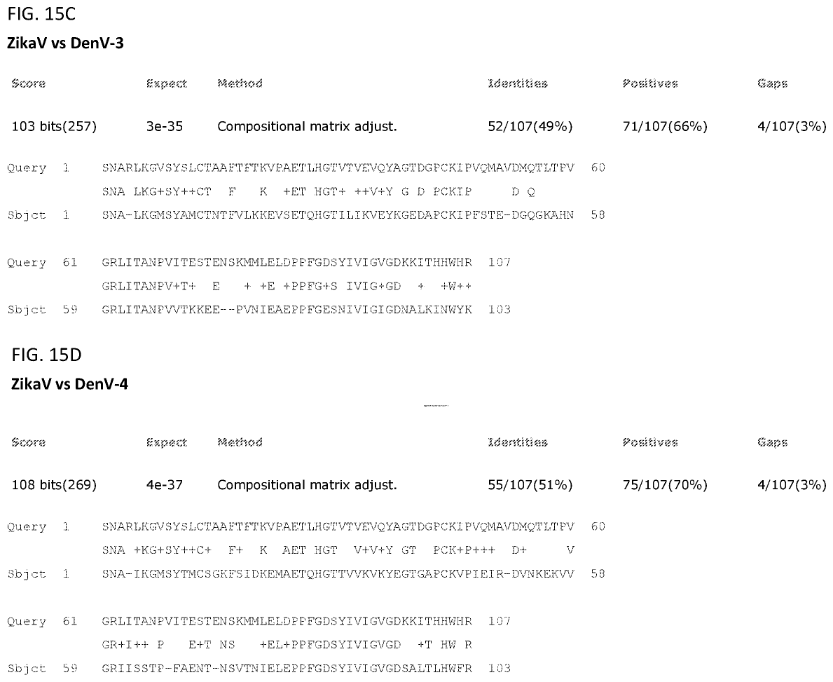

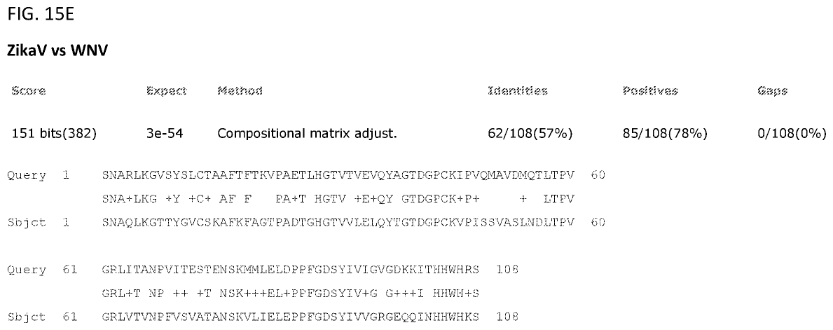

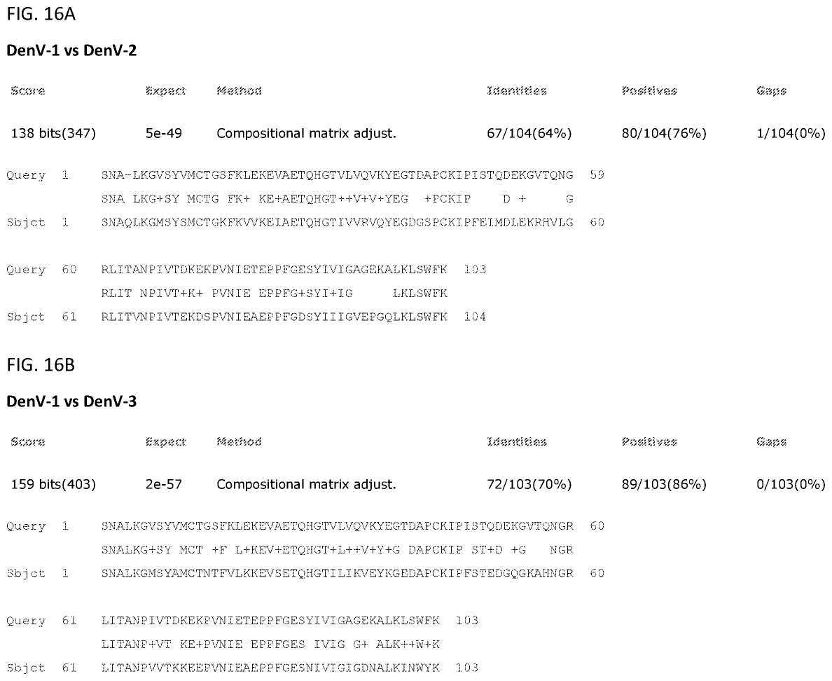

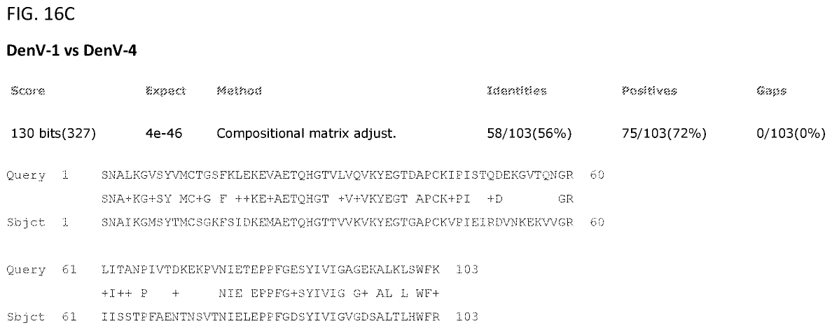

However, during size exclusion chromatographic (SEC) characterization of the tetravalent DENV-1/DENV-2/DENV-3/DENV-4 construct, a dramatic shift in the elution profiles of the antigens before and after formulation with the adjuvant EY-4-143 was observed. The tetravalent antigen mixture in the absence of EY-4-143 behaved as expected, with the elution volume of the mixture (79.6 min) corresponding to mass of 12 kDa, indicating that there was no significant aggregation (or intermolecular interactions) between the four subunit antigens. Upon addition of EY-4-143, the antigens largely eluted in the void volume (43.8 min), suggesting that the antigens were complexed with EY-4-143, and in dynamic equilibrium favoring the complexed state (43.8 min peak) over the free, uncomplexed form (79.6 min). Additional evidence for strong interactions between the antigen and adjuvant was obtained in ¹⁵N/¹H HSQC experiments, wherein the addition of EY-4-143 to ¹⁵N-labeled ZIKV antigen was found to specifically result in chemical shift perturbations of a few residues (FIG. 14).

In preliminary immunogenicity screens with the ZIKV antigen, several candidate adjuvants were examined and only EY-4-143 was found to consistently elicit neutralizing antibodies after the first boost.

### Example 6

This Example describes the immunogenicity, protective efficacy, and cross reactivity (in vitro) of MBP2-DENV-1, MBP2-DENV-2, MBP2-DENV-3, MBP2-DENV-4, MBP2-WNV, and MBP2-ZIKV antigens.

Rabbits (n=4/cohort) were immunized with 10 μg per dose MBP2-DENV-1, MBP2-DENV-2, MBP2-DENV-3, MBP2-DENV-4, MBP2-WNV, or MBP2-ZIKV antigens, as shown in Table 6, or with 10 μg each per dose of MBP2-DENV-1, MBP2-DENV-2, MBP2-DENV-3, and MBP2-DENV-4 (“Tetravalent MBP2-DENV-(1-4)”), or with 10 μg each per dose of MBP2-DENV-1, MBP2-DENV-2, MBP2-DENV-3, MBP2-DENV-4, MBP2-WNV, and MBP2-ZIKV (“Hexavalent MBP2-ZIKV/WNV/DENV-(1-4)”), each adjuvanted with EY-4-143 (100 μg/dose), as described in the Standardized Rabbit Model of Immunogenicity, to determine whether the antigens successfully induce neutralizing antibodies. Neutralizing antibodies were assessed using GFP-expressing recombinant viral particles.

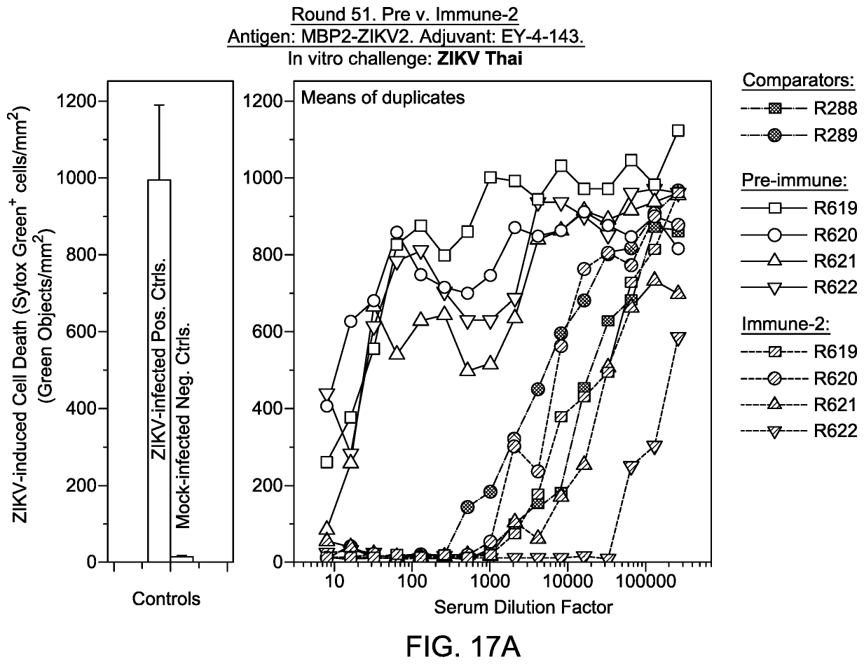

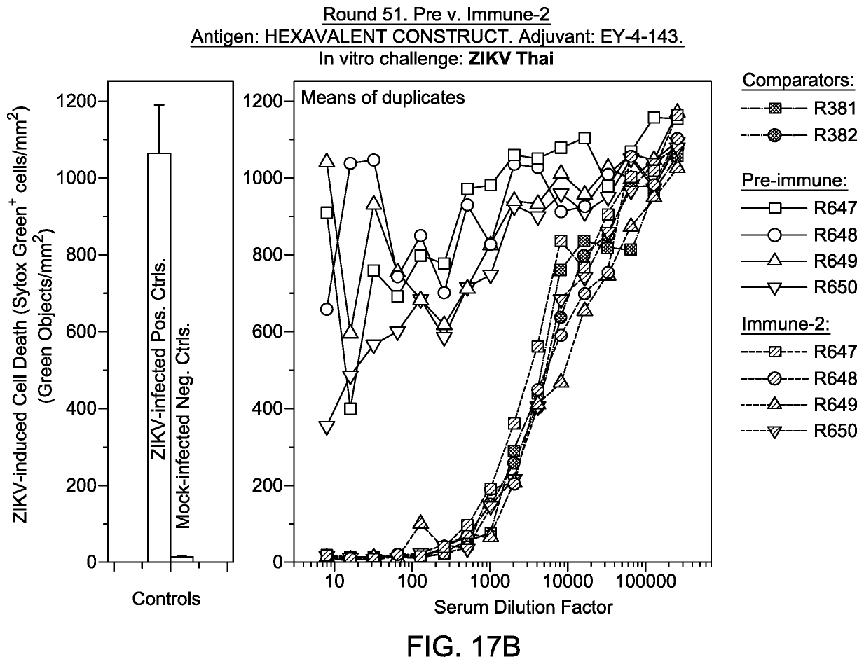

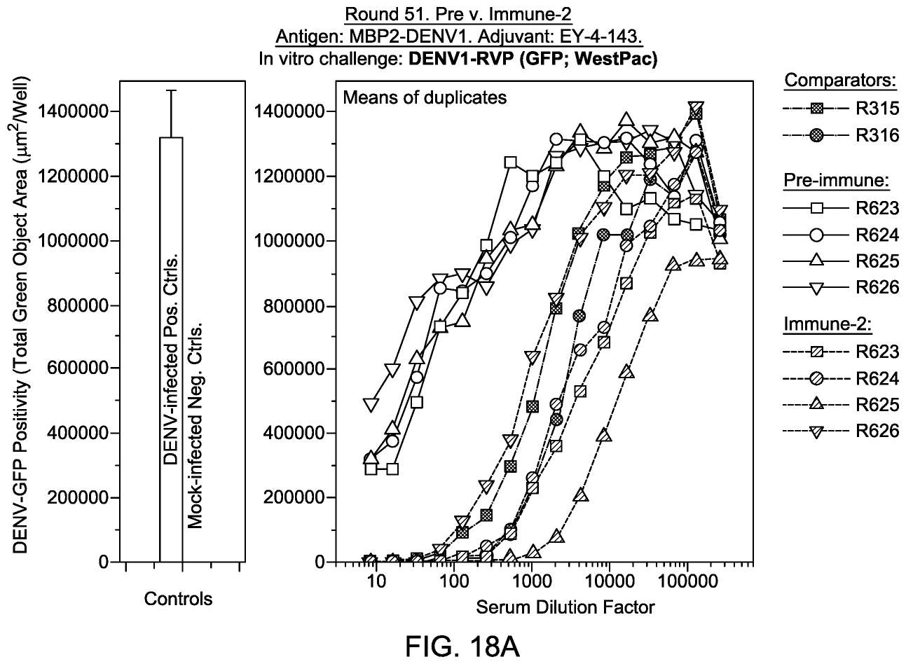

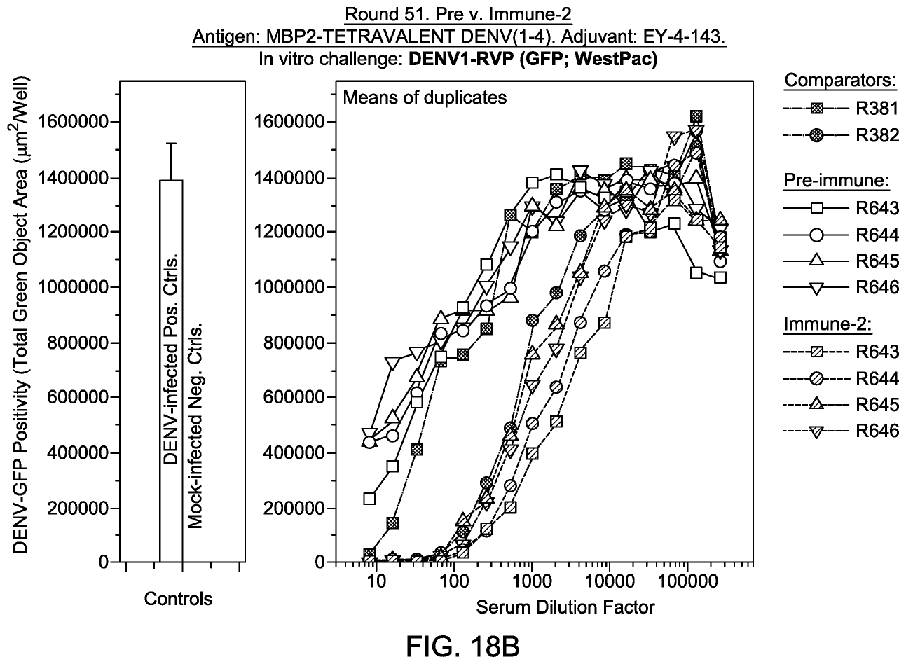

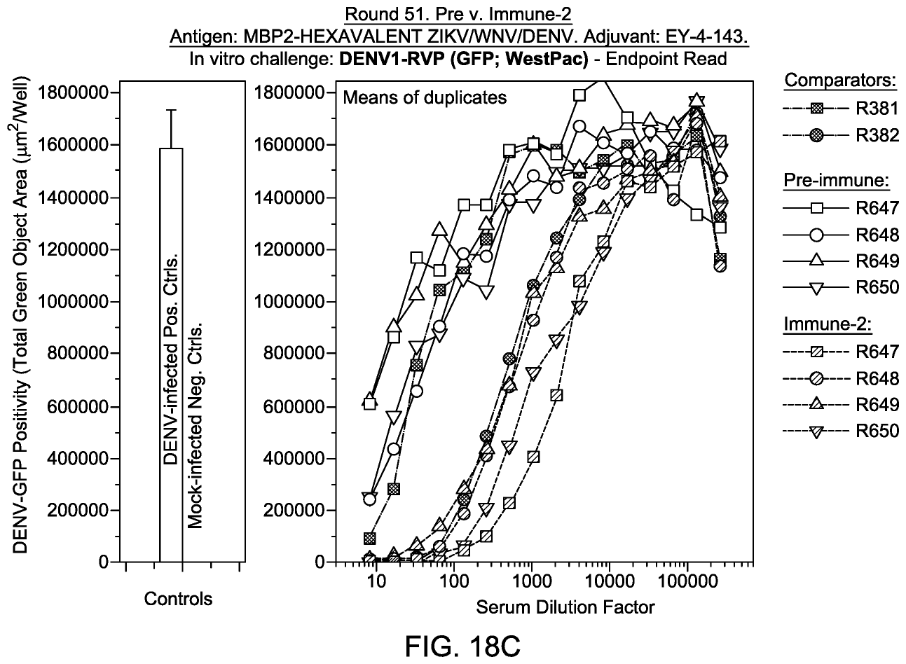

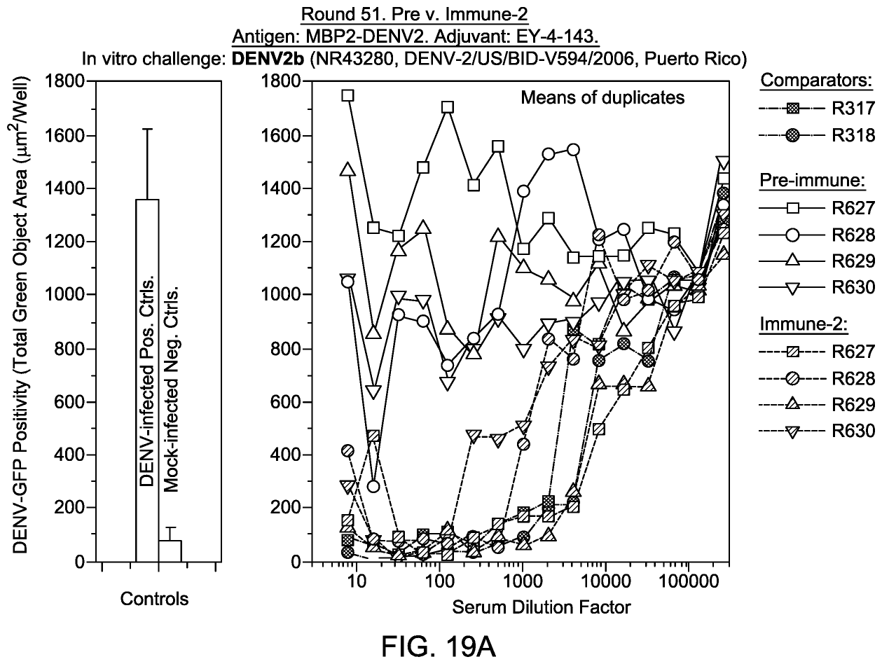

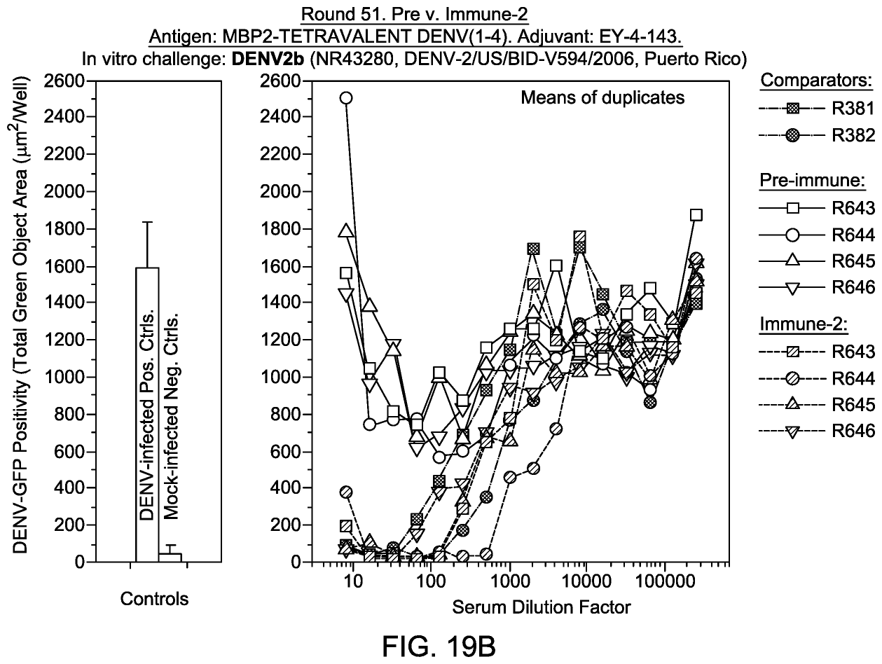

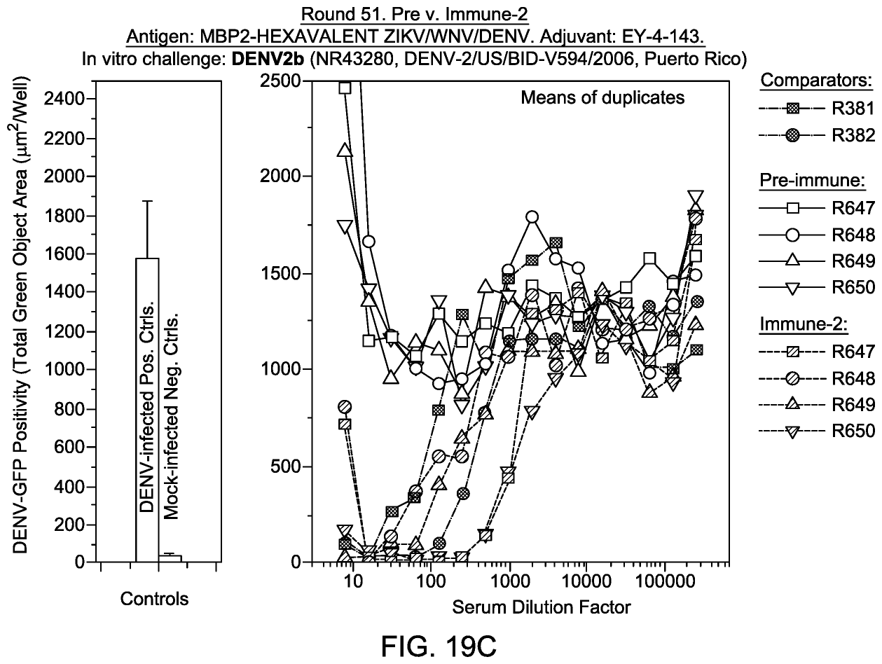

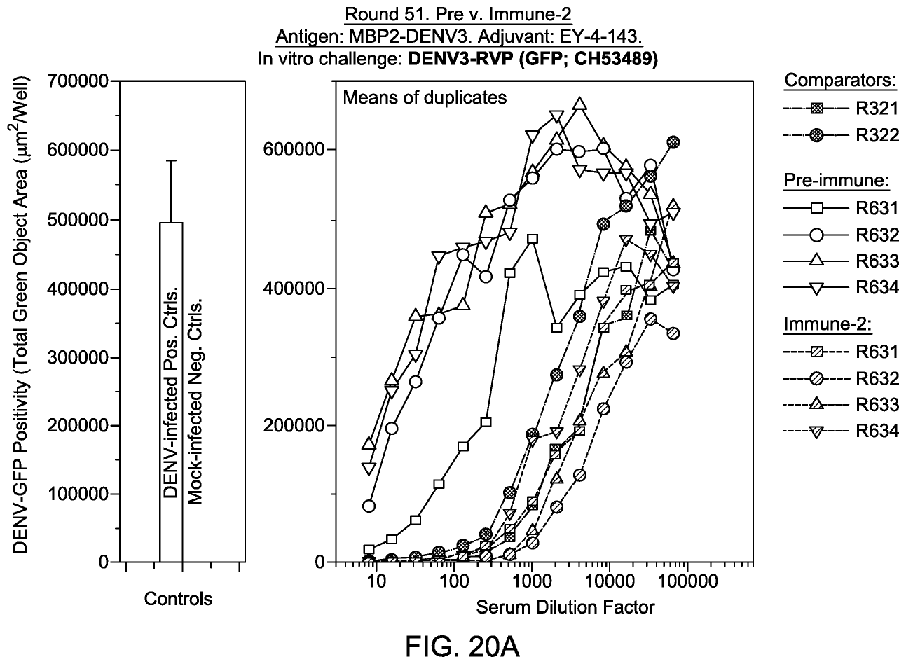

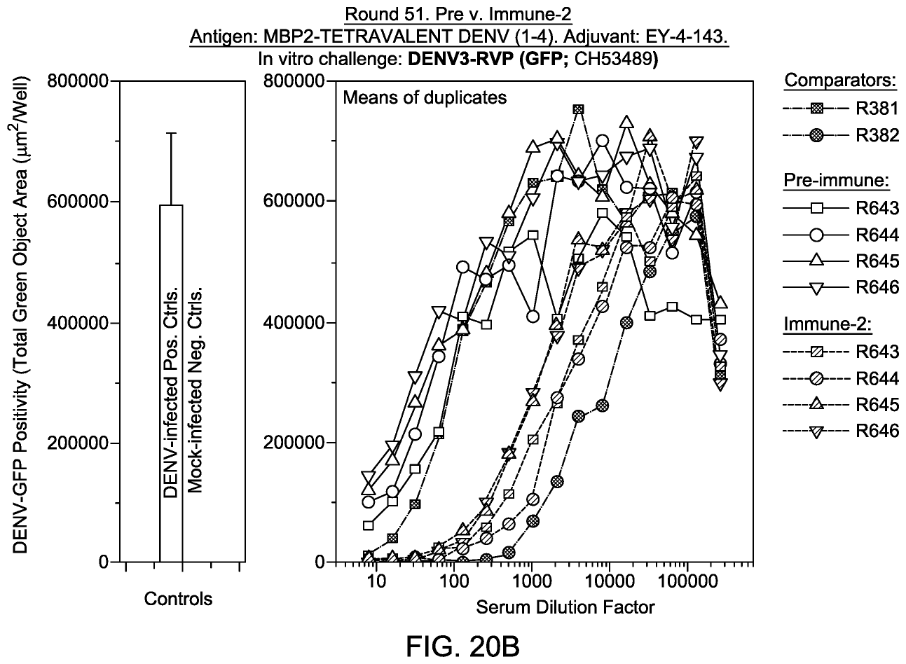

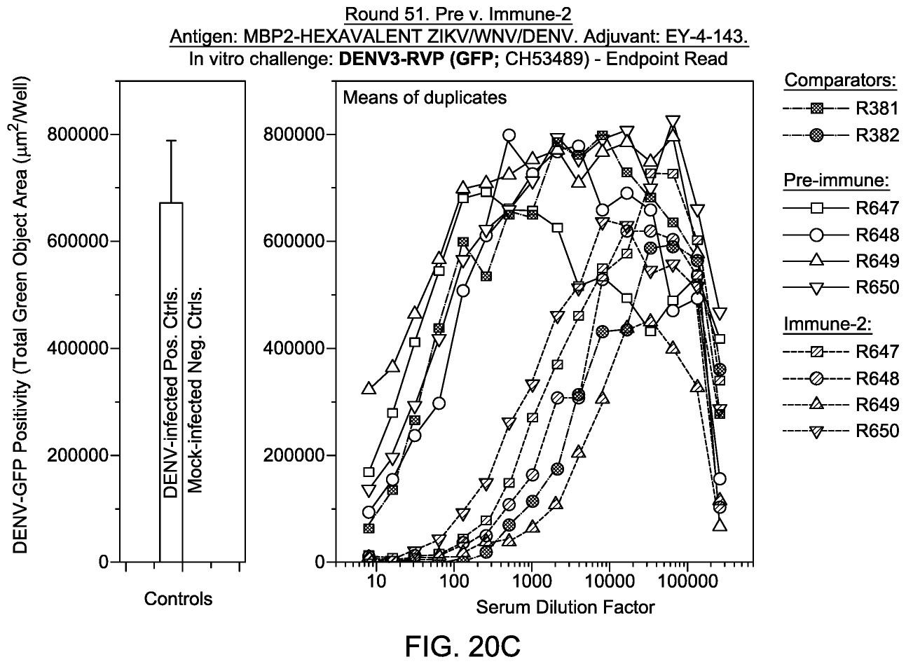

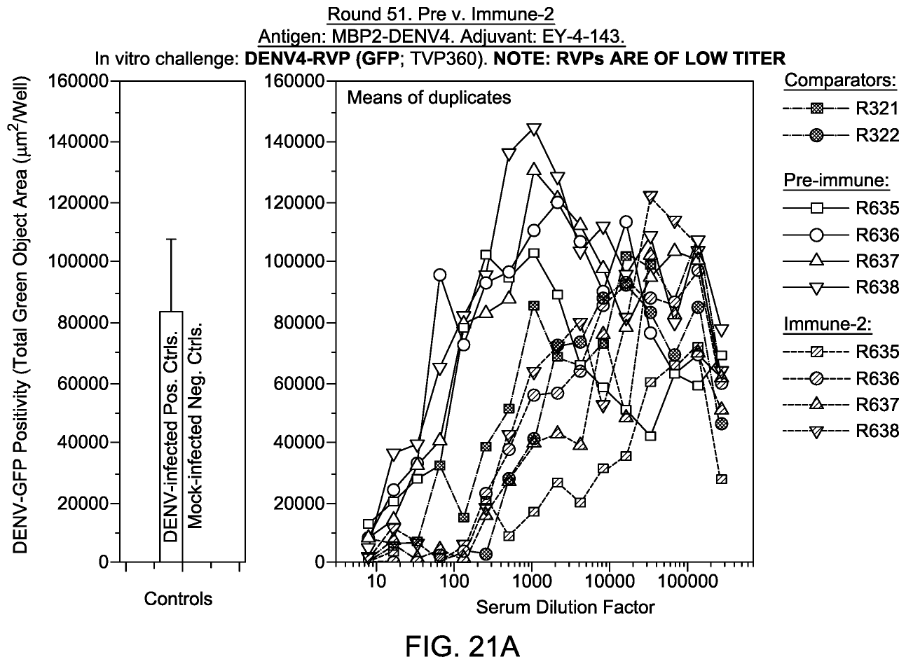

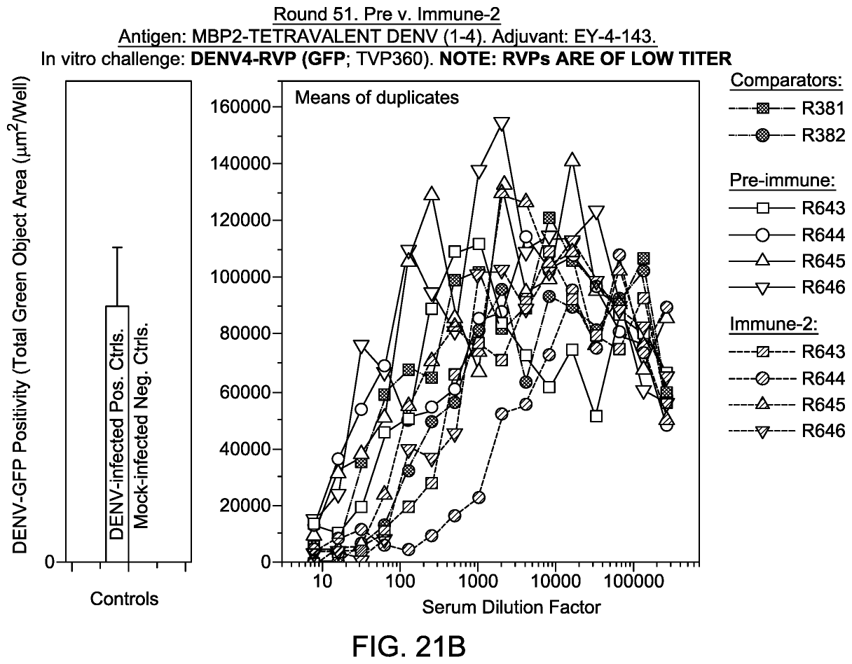

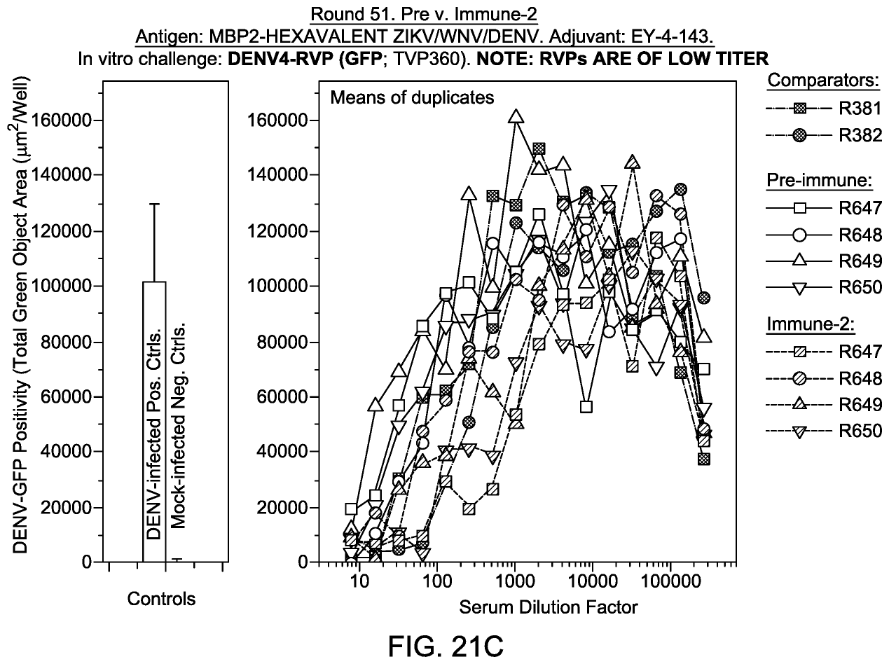

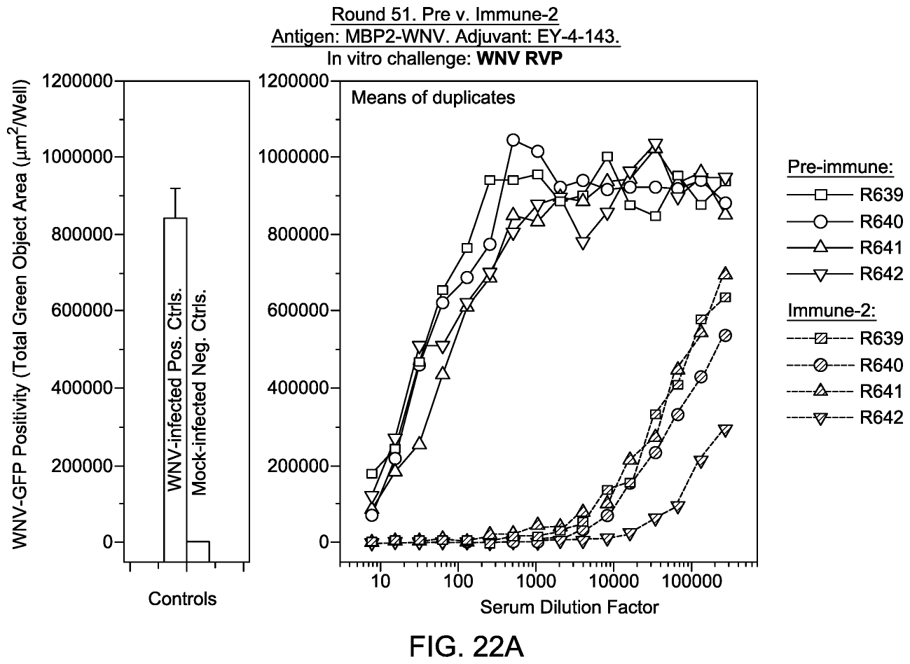

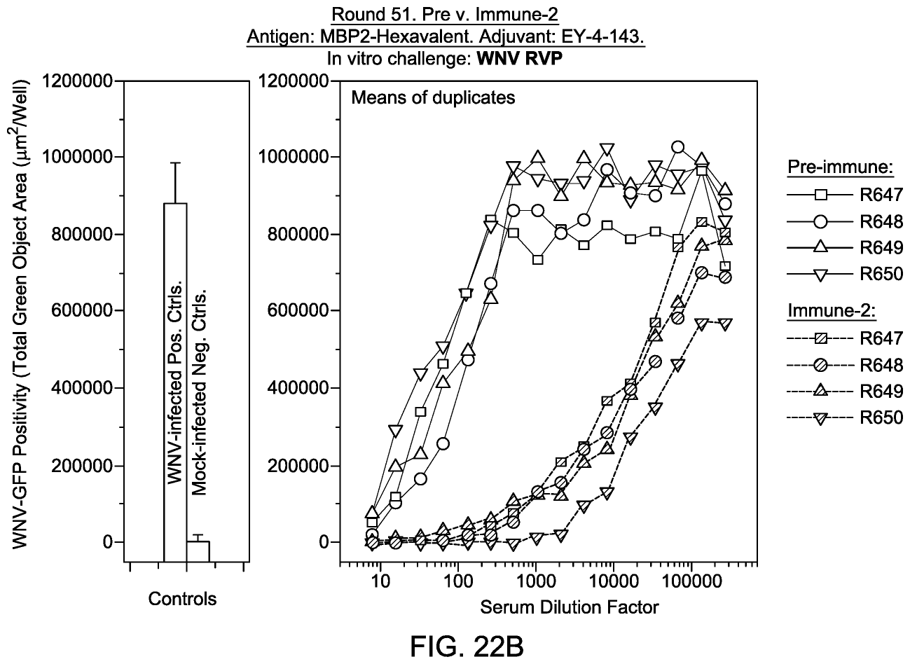

Results are shown in FIG. 17-FIG. 22. Good antibody titers were observed with each antigen, and no antigenic interference was observed in any of the vaccinated animals.

TABLE 5A

| Flavivirus | Antigen sequence | SEQ ID NO: |
|---|---|---|
| DENV-1 | QLKGMSYSMCTGKFKVVKEIAETQHGTIVVRVQYEGDGSPCKIPFEIMDLEKRHVLGRLITVNPI | 7 |
| | VTEKDSPVNIEAEPFGDSYIIIGVEPGQLKLSWFKK | |
| DENV-2 | QLKGMSYSMCTGKFKVVKEIAETQHGTIVVRVQYEGDGSPCKIPFEIMDLEKRHVLGRLITVNPI | 8 |
| | VTEKDSPVNIEAEPFGDSYIIIGVEPGQLKLSWFKK | |
| DENV-3 | LKGMSYAMCTNTFVLKKEVSETQHGTILIKVEYKGEDAPCKIPFSTEDGQGKAHNGRLITANPV | 9 |
| | VTKKEEPVNIEAEPPFGESNIVIGIGDNALKINWYKK | |
| DENV-4 | QLKGTTYGVCSKAFKFAGTPADTGHGTVVLELQYTGTDGPCKVPISSVASLNDLTPVGRLVTVN | 10 |
| | PFVSVATANSKVLIELEPPFGDSYIVVGRGEQQINHHWHKS | |
| WNV | QLKGTTYGVCSKAFKFAGTPADTGHGTVVLELQYTGTDGPCKVPISSVASLNDLTPVGRLVTVN | 11 |
| | PFVSVATANSKVLIELEPFGDSYIVVGRGEQQINHHWHKS | |
| ZIKV | RLKGVSYSLCTAAFTFTKVPAETLHGTVTVEVQYAGTDGPCKIPVQMAVDMQTLTPVGRLITAN | 12 |
| | PVITESTENSKMMLELDPPFGDSYIVIGVGDKKITHHWHRS | |

TABLE 5B

| Protein Tag | Sequence | SEQ ID NO: |
|---|---|---|
| MBP | KIEEGKLVIWINGDKGYNGLAEVGKKFEKDTGIKVTVEHPDKLEEKFPQVAATGDGPDIIFWAH | 13 |
| | DRFGGYAQSGLLAEITPDKAFQDKLYPFTWDAVRYNGKLIAYPIAVEALSLIYNKDLLPNPPKT | |
| | WEEIPALDKELKAKGKSALMFNLQEPYFTWPLIAADGGYAFKYENGKYDIKDVGVDNAGAKA | |
| | GLTFLVDLIKNKHMNADTDYSIAEAAFNKGETAMTINGPWAWSNIDTSKVNYGVTVLPTFKGQ | |
| | PSKPFVGVLSAGINAASPNKELAKEFLENYLLTDEGLEAVNKDKPLGAVALKSYEEELAKDPRIA | |
| | ATMENAQKGEIMPNIPQMASAFWYAVRTAVINAASGRQTVDEALKDAQT | |
| SGp | LAEAKVLANRELDKYGVSDYYKNLINNAKTVEGVKALIDEILAALPK | 14 |

TABLE 6
A 6-His leader sequence (SEQ ID NO: 15) is italicized. STS, shown in underlined, bold text, indicates a conjoining tripeptide used to
concatenate the poly-his tag with the rest of the sequence The MBP sequence is in plain text (not bold or underlined) The Flaviviral
antigen is shown in bold. GT, underlined, indicates a transition dipeptide

| | | |
|---|---|---|
| MBP2- | MHHHHHHSTSKIEEGKLVIWINGDKGYNGLAEVGKKFEKDTGIKVTVEHPDKLEEKFPQVAATGDGPDI | SEQ ID NO: 32 |
| DENV-1 | IFWAHDRFGGYAQSGLLAEITPDKAFQDKLYPFTWDAVRYNGKLIAYPIAVEALSLIYNKDLLPNPPKT | |
| | WEEIPALDKELKAKGKSALMFNLQEPYFRWPLIAADGGYAFKYENGKYDIKDVGVDNAGAKAGLTFL | |
| | VDLIKNKHMNADTDYSIAEAAFNKGETAMTINGPWAWSNIDTSKVNYGVTVLPTFKGQPSKPFVGVLS | |
| | AGINAASPNKELAKEFLENYLLTDEGLEAVNKDKPLGAVALKSYEEELAKDPRIAATMENAQKGEIMPN | |
| | IPQMSAFWYAVRTAVINAASGRQTVDEALKDAQTGTQLKGMSYSMCTGKFKVVKEIAETQHGTIVV | |
| | RVQYEGDGSPCKIPFEIMDLEKRHVLGRLITVNPIVTEKDSPVNIEAEPPFGDSYIIIGVEPGQLKLS | |
| | WFKK | |
| MBP2- | MHHHHHHSTSKIEEGKLVIWINGDKGYNGLAEVGKKFEKDTGIKVTVEHPDKLEEKFPQVAATGDGPDI | SEQ ID NO: 33 |
| DENV-2 | IFWAHDRFGGYAQSGLLAEITPDKAFQDKLYPFTWDAVRYNGKLIAYPIAVEALSLIYNKDLLPNPPKT | |
| | WEEIPALDKELKAKGKSALMFNLQEPYFRWPLIAADGGYAFKYENGKYDIKDVGVDNAGAKAGLTFL | |
| | VDLIKNKHMNADTDYSIAEAAFNKGETAMTINGPWAWSNIDTSKVNYGVTVLPTFKGQPSKPFVGVLS | |
| | AGINAASPNKELAKEFLENYLLTDEGLEAVNKDKPLGAVALKSYEEELAKDPRIAATMENAQKGEIMPN | |
| | IPQMSAFWYAVRTAVINAASGRQTVDEALKDAQTGTQLKGMSYSMCTGKFKVVKEIAETQHGTIVV | |
| | RVQYEGDGSPCKIPFEIMDLEKRHVLGRLITVNPIVTEKDSPVNIEAEPPFGDSYIIIGVEPGQLKLS | |
| | WFKK | |
| MBP2- | MHHHHHHSTSKIEEGKLVIWINGDKGYNGLAEVGKKFEKDTGIKVTVEHPDKLEEKFPQVAATGDGPDI | SEQ ID NO: 34 |
| DENV-3 | IFWAHDRFGGYAQSGLLAEITPDKAFQDKLYPFTWDAVRYNGKLIAYPIAVEALSLIYNKDLLPNPPKT | |
| | WEEIPALDKELKAKGKSALMFNLQEPYFRWPLIAADGGYAFKYENGKYDIKDVGVDNAGAKAGLTFL | |
| | VDLIKNKHMNADTDYSIAEAAFNKGETAMTINGPWAWSNIDTSKVNYGVTVLPTFKGQPSKPFVGVLS | |
| | AGINAASPNKELAKEFLENYLLTDEGLEAVNKDKPLGAVALKSYEEELAKDPRIAATMENAQKGEIMPN | |
| | IPQMSAFWYAVRTAVINAASGRQTVDEALKDAQTGTLKGMSYAMCTNTFVLKKEVSETQHGTILIK | |
| | VEYKGEDAPCKIPFSTEDGQGKAHNGRLITANPVVTKKEEPVNIEAEPPFGESNIVIGIGDNALKIN | |
| | WFKK | |
| MBP2- | MHHHHHHSTSKIEEGKLVIWINGDKGYNGLAEVGKKFEKDTGIKVTVEHPDKLEEKFPQVAATGDGPDI | SEQ ID NO: 35 |
| DENV-4 | IFWAHDRFGGYAQSGLLAEITPDKAFQDKLYPFTWDAVRYNGKLIAYPIAVEALSLIYNKDLLPNPPKT | |
| | WEEIPALDKELKAKGKSALMFNLQEPYFRWPLIAADGGYAFKYENGKYDIKDVGVDNAGAKAGLTFL | |
| | VDLIKNKHMNADTDYSIAEAAFNKGETAMTINGPWAWSNIDTSKVNYGVTVLPTFKGQPSKPFVGVLS | |
| | AGINAASPNKELAKEFLENYLLTDEGLEAVNKDKPLGAVALKSYEEELAKDPRIAATMENAQKGEIMPN | |
| | IPQMSAFWYAVRTAVINAASGRQTVDEALKDAQTGTQLKGTTYGVCSKAFKFAGTPADTGHGTVVL | |
| | ELQYTGTDGPCKVPISSVASLNDLTPVGRLVTVNPFVSVSTANSKVLIELEPPFGDSYIVVGRGEQQ | |
| | INHHWHKS | |
| MBP2- | MHHHHHHSTSKIEEGKLVIWINGDKGYNGLAEVGKKFEKDTGIKVTVEHPDKLEEKFPQVAATGDGPDI | SEQ ID NO: 36 |
| WNV | IFWAHDRFGGYAQSGLLAEITPDKAFQDKLYPFTWDAVRYNGKLIAYPIAVEALSLIYNKDLLPNPPKT | |
| | WEEIPALDKELKAKGKSALMFNLQEPYFRWPLIAADGGYAFKYENGKYDIKDVGVDNAGAKAGLTFL | |
| | VDLIKNKHMNADTDYSIAEAAFNKGETAMTINGPWAWSNIDTSKVNYGVTVLPTFKGQPSKPFVGVLS | |
| | AGINAASPNKELAKEFLENYLLTDEGLEAVNKDKPLGAVALKSYEEELAKDPRIAATMENAQKGEIMPN | |
| | IPQMSAFWYAVRTAVINAASGRQTVDEALKDAQTGTQLKGTTYGVCSKAFKFAGTPADTGHGTVVL | |
| | ELQYTGTDGPCKVPISSVASLNDLTPVGRLVTVNPFVSVSTANSKVLIELEPPFGDSYIVVGRGEQQ | |
| | INHHWHKS | |
| MBP2- | MHHHHHHSTSKIEEGKLVIWINGDKGYNGLAEVGKKFEKDTGIKVTVEHPDKLEEKFPQVAATGDGPDI | SEQ ID NO: 37 |
| ZIKV | IFWAHDRFGGYAQSGLLAEITPDKAFQDKLYPFTWDAVRYNGKLIAYPIAVEALSLIYNKDLLPNPPKT | |
| | WEEIPALDKELKAKGKSALMFNLQEPYFRWPLIAADGGYAFKYENGKYDIKDVGVDNAGAKAGLTFL | |
| | VDLIKNKHMNADTDYSIAEAAFNKGETAMTINGPWAWSNIDTSKVNYGVTVLPTFKGQPSKPFVGVLS | |
| | AGINAASPNKELAKEFLENYLLTDEGLEAVNKDKPLGAVALKSYEEELAKDPRIAATMENAQKGEIMPN | |
| | IPQMSAFWYAVRTAVINAASGRQTVDEALKDAQTGTRLKGVSYSLCTAAFTFTKVPAETLHGTVTVE | |
| | VQYAGTDGPCKIPVQMAVDMQTLTPVGRLITANPVITESTENSKMMLELDPPFGDSYIVIGVGDKK | |
| | ITHHWHRS | |

### Example 7

This Example describes the immunogenicity, protective efficacy, and cross reactivity (in vitro) of SGp(L)-DENV-1, SGp(L)-DENV-2, SGp(L)-DENV-3, SGp(L)-DENV-4, SGp(L)-WNV, and SGp(L)-ZIKV antigens.

Each antigen includes a portion of the Strepocococcal G protein (SGP) at the N-terminus. The SGP portion was selected for its good binding to albumin (human, non-human primate, pig, rabbit, rat, mouse), but not to immunoglobulins. Without wishing to be bound by theory, it is believed that the binding to albumin facilitates delivery of the antigen to the lymph nodes while the abrogation of binding to immunoglobulins obviates problems with affinity maturation of the resultant antibody response. (See, for example, Nilvebrant et al. Computational and Structural Biotech. J. 2013, 6(7):e201303009; Lejon et al., J. Biol. Chem. 2004, 279(41):42924-42928; Jonsson et al. Protein Engineering, Design and Selection 2008, 21(8):515-527.)

Rabbits (n=4/cohort) were immunized with 10 μg each antigen per dose as described in the Standardized Rabbit Model of Immunogenicity modified as shown in Table 8. Antigens are shown in Table 7; each dose was adjuvanted with EY-4-143 (100 μg/dose).

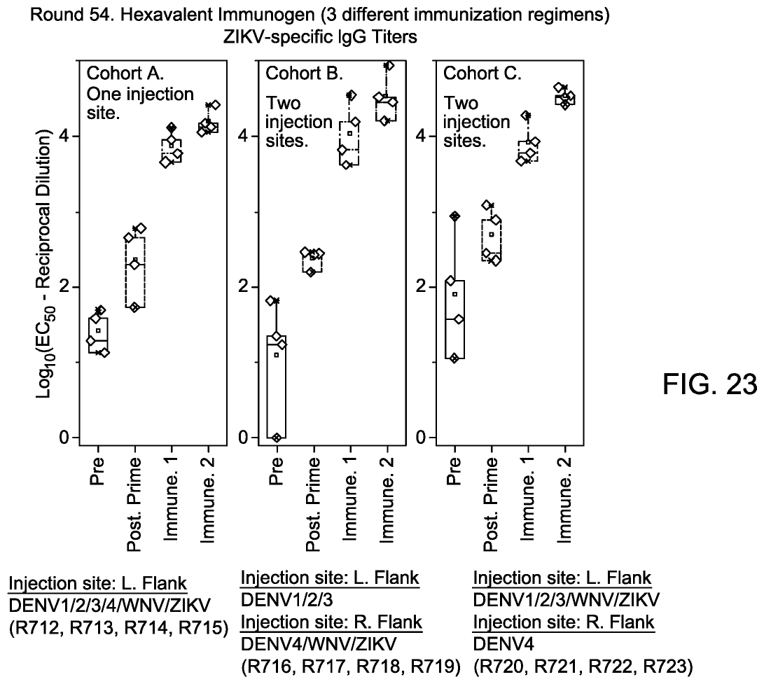

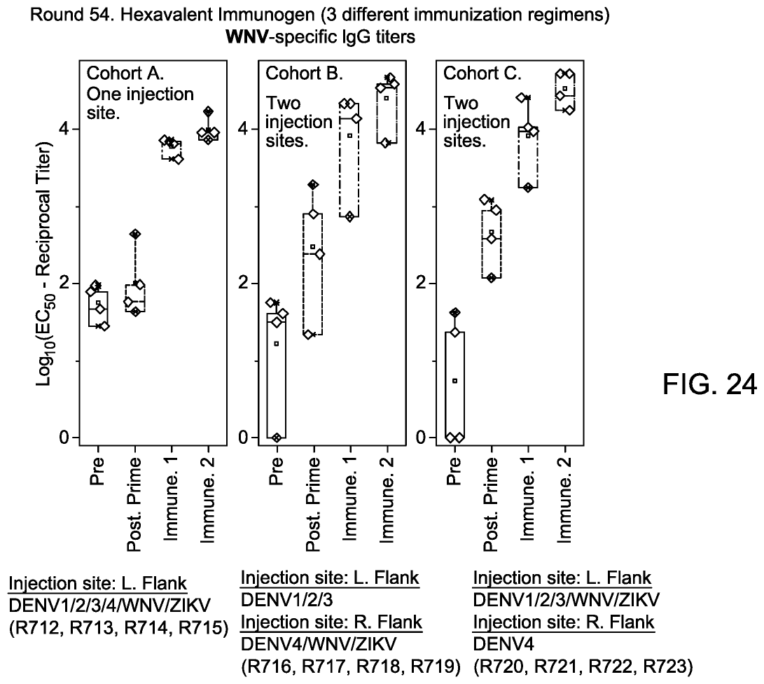

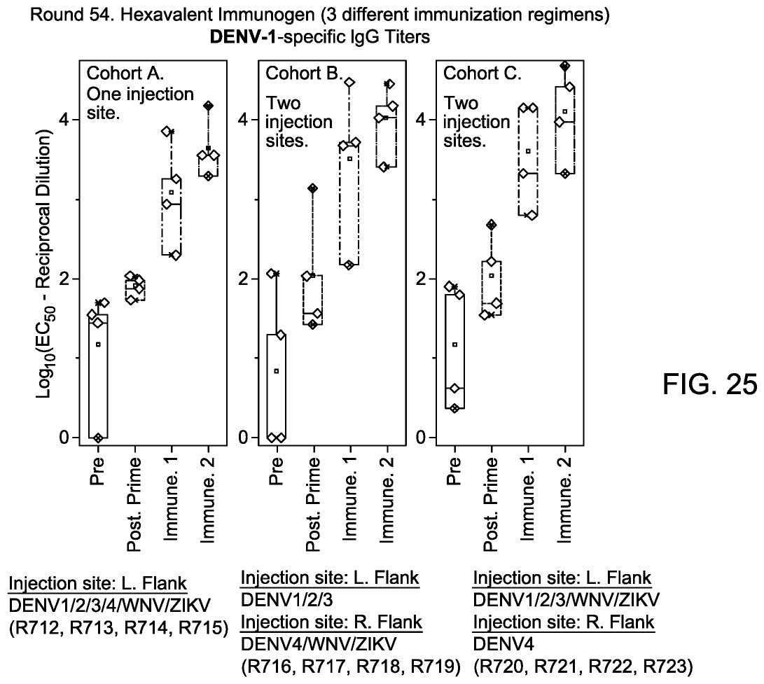

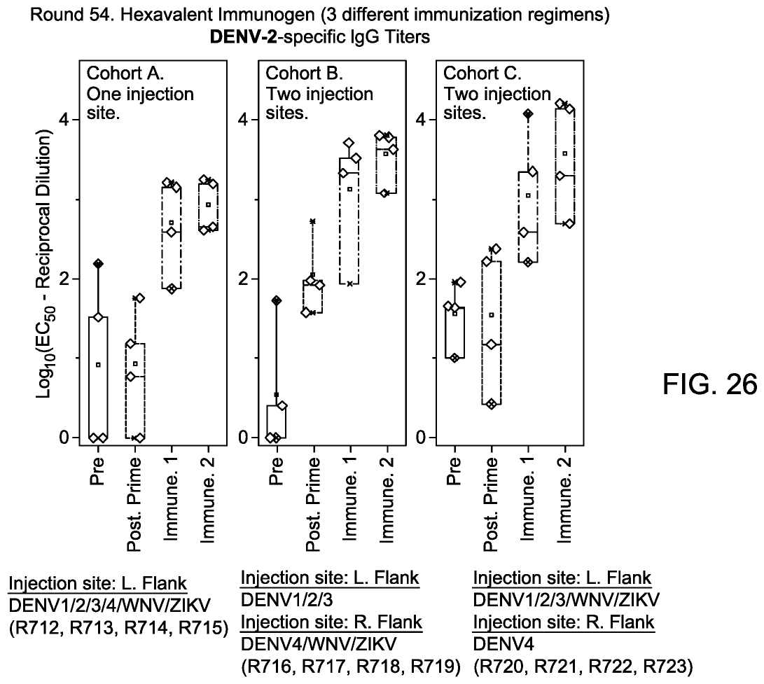

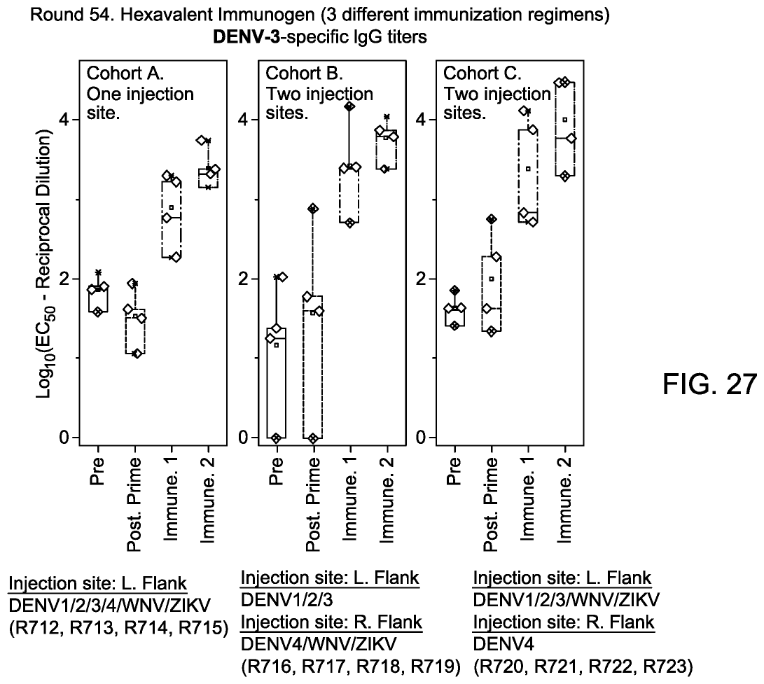

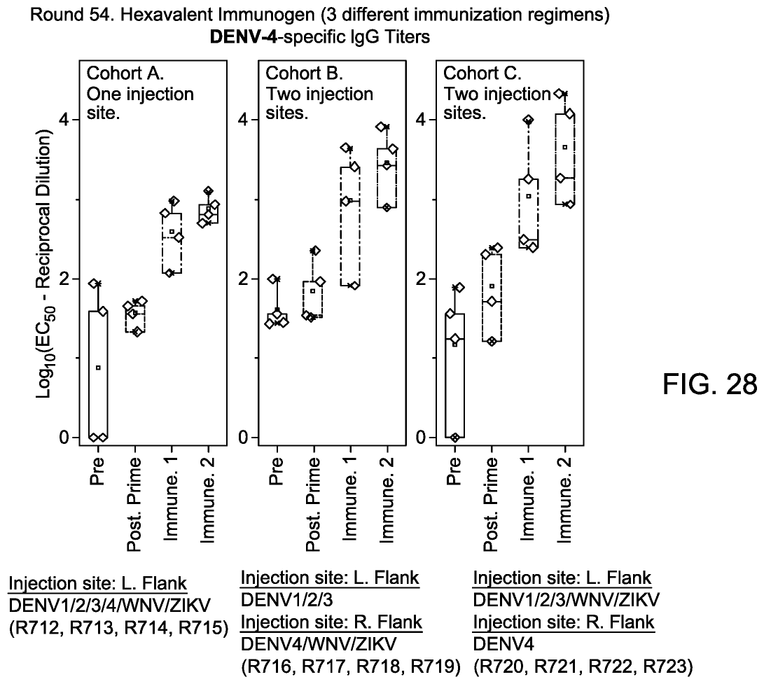

A rise in antibody titers, as measured by ELISA was comparable in cohorts A, B, and C (statistically insignificant differences; results are shown in FIG. 23-FIG. 28.

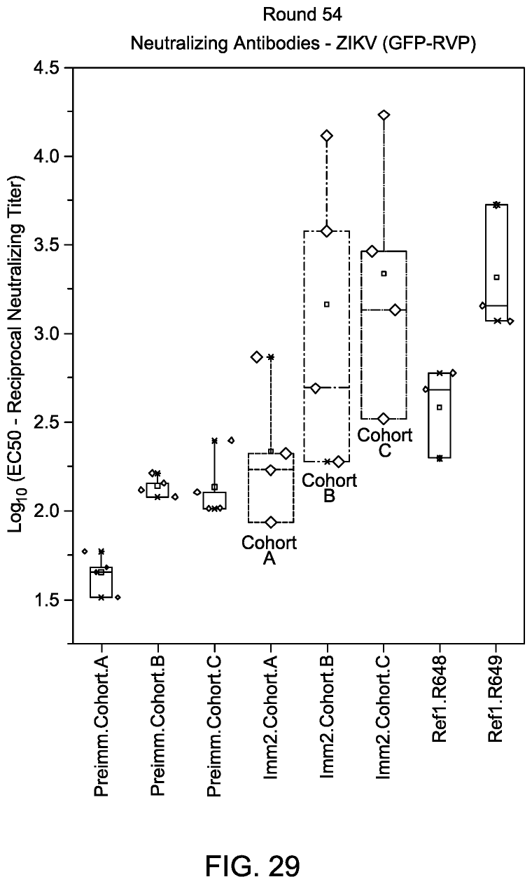

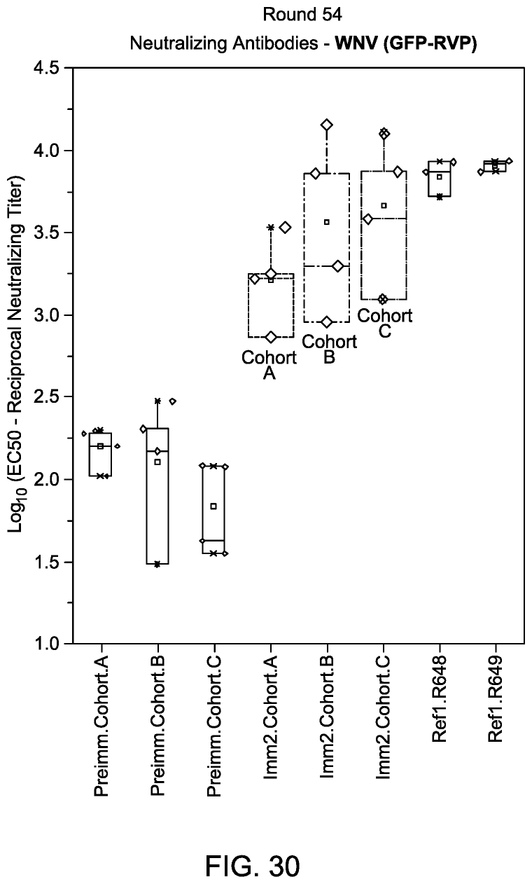

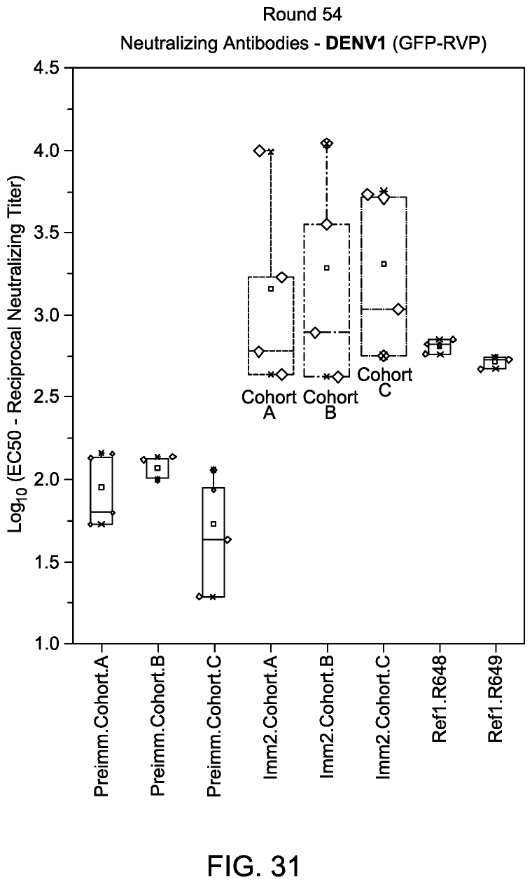

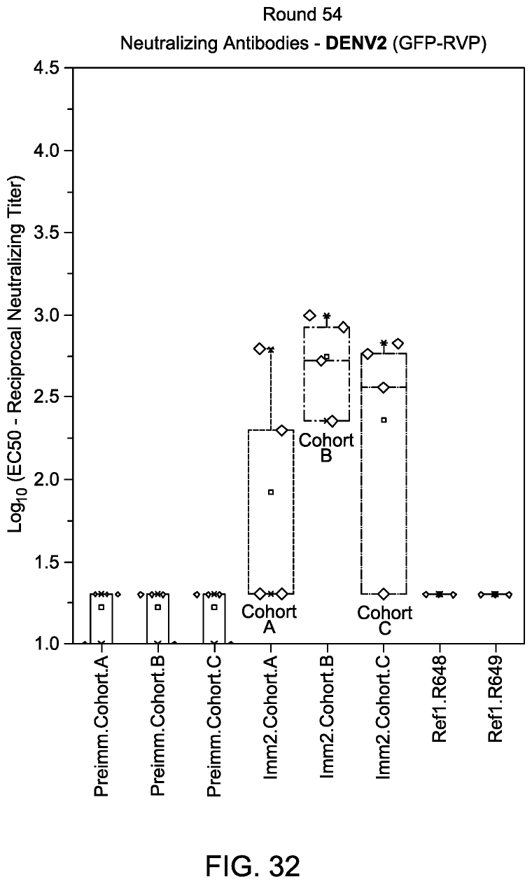

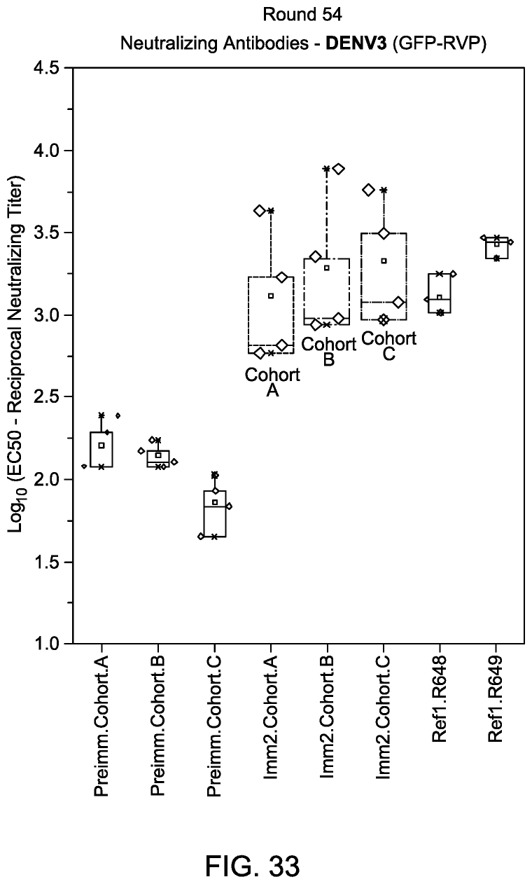

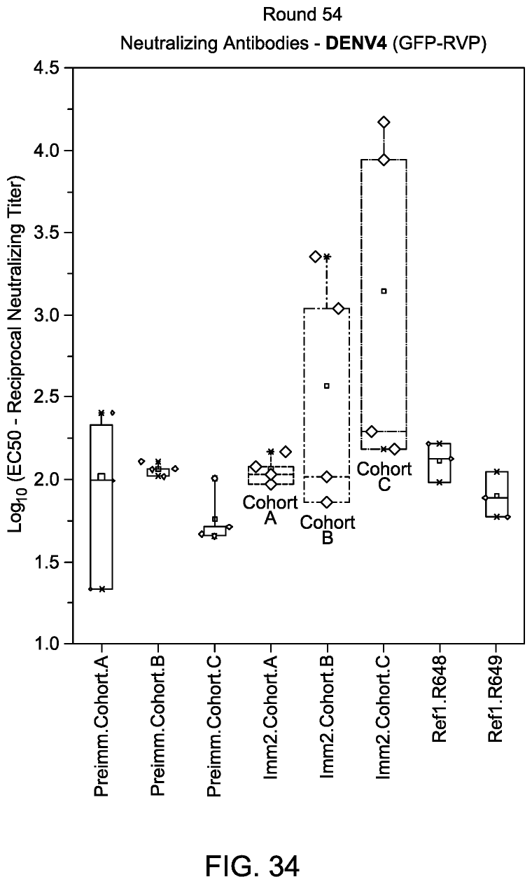

Neutralizing antibodies were assessed using GFP-expressing recombinant viral particles. Results are shown in FIG. 29-FIG. 34.

TABLE 7
A 6-His leader sequence (SEQ ID NO: 15) is italicized. STS, shown in underlined gray text,
indicates a conjoining tripeptide used to concatenate the poly-his tag with the rest of the sequence
The SGp(L) sequence is in black text (not bold or underlined) The Flaviviral antigen is shown in
bold. GT, underlined, indicates a transition dipeptide.

| | | |
|---|---|---|
| SGp(L)- | MHHHHHHSTSLAEAKVLANRELDKYGVSDYYKNLINNAKTVEGVKA | SEQ ID |
| DENV-1 | LIDEILAALPKGTLKGVSYVMCTGSFKLEKEVAETQHGTVLVQVKY | NO: 38 |
| | EGTDAPCKIPISTQDEKGVTQNGRLITANPIVTDKEKPVNIETEPPF | |
| | GESYIVIGAGEKALKLSWFK | |
| SGp(L)- | MHHHHHHSTSLAEAKVLANRELDKYGVSDYYKNLINNAKTVEGVKA | SEQ ID |
| DENV-2 | LIDEILAALPKGTQLKGMSYSMCTGKFKVVKEIAETQHGTIVVRVQ | NO: 39 |
| | YEGDGSPCKIPFEIMDLEKRHVLGRLITVNPIVTEKDSPVNIEAEPP | |
| | FGDSYIIIGVEPGQLKLSWFKK | |
| SGp(L)- | MHHHHHHSTSLAEAKVLANRELDKYGVSDYYKNLINNAKTVEGVKA | SEQ ID |
| DENV-3 | LIDEILAALPKGTLKGMSYAMCTNTFVLKKEVSETQHGTILIKVEYK | NO: 40 |
| | GEDAPCKIPFSTEDGQGKAHNGRLITANPVVTKKEEPVNIEAEPPF | |
| | GESNIVIGIGDNALKINWYKK | |
| SGp(L)- | MHHHHHHSTSLAEAKVLANRELDKYGVSDYYKNLINNAKTVEGVKA | SEQ ID |
| DENV-4 | LIDEILAALPKGTIKGMSYTMCSGKFSIDKEMAETQHGTTVVKVKY | NO: 41 |
| | EGTGAPCKVPIEIRDVNKEKVVGRIISSTPFAENTNSVTNIELEPPFG | |
| | DSYIVIGVGDSALTLHWFR | |
| SGp(L)- | MHHHHHHSTSLAEAKVLANRELDKYGVSDYYKNLINNAKTVEGVKA | SEQ ID |
| WNV | LIDEILAALPKGTQLKGTTYGVCSKAFKFAGTPADTGHGTVVLELQ | NO: 42 |
| | YTGTDGPCKVPISSVASLNDLTPVGRLVTVNPFVSVATANSKVLIEL | |
| | EPPFGDSYIVVGRGEQQINHHWHKS | |
| SGp(L)- | MHHHHHHSTSLAEAKVLANRELDKYGVSDYYKNLINNAKTVEGVKA | SEQ ID |
| ZIKV | LIDEILAALPKGTRLKGVSYSLCTAAFTFTKVPAETLHGTVTVEVQY | NO: 43 |
| | AGTDGPCKIPVQMAVDMQTLTPVGRLITANPVITESTENSKMMLE | |
| | LDPPFGDSYIVIGVGDKKITHHWHRS | |

TABLE 8

| | |
|---|---|
| Cohort A | Single injection (n = 4) |
| | All 6 antigens (of Table 7) - Left flank |
| Cohort B | Two separate injections (n = 4) |
| | Left flank: SGp(L)-DENV-l/SGp(L)-DENV-2/SGp(L)-DENV-3 |
| | Right flank: SGp(L)-DENV-4/SGp(L)-WNV/SGp(L)-ZIKV |
| Cohort C | Two separate injections (n = 4) |
| | Left flank: SGp(L)-DENV-l/SGp(L)-DENV-2/SGp(L)-DENV-3/ |
| | SGp(L)-WNV/SGp(L)-ZIKV |
| | Right flank: SGp(L)-DENV-4 |

### REFERENCES

1. Shiwani, H. A.; Pharithi, R. B.; Khan, B.; Egom, C. B.; Kruzliak, P.; Maher, V.; Egom, E. E. An update on the 2014 Ebola outbreak in Western Africa. Asian Pacific J. Trop. Med. 2017, 10, 6-10.

2. Coltart, C. E.; Lindsey, B.; Ghinai, I.; Johnson, A. M.; Heymann, D. L. The Ebola outbreak, 2013-2016: old lessons for new epidemics. Philos. Trans. Roy.Soc. London. Series B, Biological sciences 2017, 372.

3. Luquero, F. J.; Rondy, M.; Boncy, J.; Munger, A.; Mekaoui, H.; Rymshaw, E.; Page, A. L.; Toure, B.; Degail, M. A.; Nicolas, S.; Grandesso, F.; Ginsbourger, M.; Polonsky, J.; Alberti, K. P.; Terzian, M.; Olson, D.; Porten, K.; Ciglenecki, I. Mortality Rates during Cholera Epidemic, Haiti, 2010-2011. Emerg. Infect. Dis. 2016, 22, 410-416.

4. Farmer, P.; Almazor, C. P.; Bahnsen, E. T.; Barry, D.; Bazile, J.; Bloom, B. R.; Bose, N.; Brewer, T.; Calderwood, S. B.; Clemens, J. D.; Cravioto, A.; Eustache, E.; Jerome, G.; Gupta, N.; Harris, J. B.; Hiatt, H. H.; Holstein, C.; Hotez, P. J.; Ivers, L. C.; Kerry, V. B.; Koenig, S. P.; Larocque, R. C.; Leandre, F.; Lambert, W.; Lyon, E.; Mekalanos, J. J.; Mukherjee, J. S.; Oswald, C.; Pape, J. W.; Gretchko Prosper, A.; Rabinovich, R.; Raymonville, M.; Rejouit, J. R.; Ronan, L. J.; Rosenberg, M. L.; Ryan, E. T.; Sachs, J. D.; Sack, D. A.; Surena, C.; Suri, A. A.; Ternier, R.; Waldor, M. K.; Walton, D.; Weigel, J. L. Meeting cholera's challenge to Haiti and the world: a joint statement on cholera prevention and care. PLoS Neglect. Trop. Dis. 2011, 5, e1145.

5. Nam, H. S.; Park, J. W.; Ki, M.; Yeon, M. Y.; Kim, J.; Kim, S. W. High fatality rates and associated factors in two hospital outbreaks of MERS in Daejeon, the Republic of Korea. Int. J. Infect. Dis. 2017, 58, 37-42.

6. Kang, C. K.; Song, K. H.; Choe, P. G.; Park, W. B.; Bang, J. H.; Kim, E. S.; Park, S. W.; Kim, H. B.; Kim, N. J.; Cho, S. I.; Lee, J. K.; Oh, M. D. Clinical and Epidemiologic Characteristics of Spreaders of Middle East Respiratory Syndrome Coronavirus during the 2015 Outbreak in Korea. J. Korean Med. Sci. 2017, 32, 744-749.

7. Torre, J. A.; Benevides, G. N.; de Melo, A. M.; Ferreira, C. R. Pertussis: the resurgence of a public health threat. Autopsy & Case Reports 2015, 5, 9-16.

8. Tan, T.; Dalby, T.; Forsyth, K.; Halperin, S. A.; Heininger, U.; Hozbor, D.; Plotkin, S.; Ulloa-Gutierrez, R.; Wirsing von Konig, C. H. Pertussis Across the Globe: Recent Epidemiologic Trends From 2000 to 2013. Ped. Infect. Dis. 2015, 34, e222-232.

9. Sealey, K. L.; Belcher, T.; Preston, A. Bordetella pertussis epidemiology and evolution in the light of pertussis resurgence. Infection, genetics and evolution: J. Molec. Epidem. Evol. Genet. Infect. Dis. 2016, 40, 136-143.

10. Locht, C. Pertussis: Where did we go wrong and what can we do about it? J. Infect. 2016, 72 Suppl, S34-40.

11. Sabbe, M.; Vandermeulen, C. The resurgence of mumps and pertussis. Human Vacc. Immunother. 2016, 12, 955-959.

12. Donahue, M.; Schneider, A.; Ukegbu, U.; Shah, M.; Riley, J.; Weigel, A.; James, L.; Wittich, K.; Quinlisk, P.; Cardemil, C. Notes from the Field: Complications of Mumps During a University Outbreak Among Students Who Had Received 2 Doses of Measles-Mumps-Rubella Vaccine—Iowa, July 2015-May 2016. MMWR. 2017, 66, 390-391.

13. Younger, D. S. Epidemiology of Lyme Neuroborreliosis. Neurologic clinics 2016, 34, 875-886.

14. Schotthoefer, A. M.; Frost, H. M. Ecology and Epidemiology of Lyme Borreliosis. Clin. Lab. Med. 2015, 35, 723-743.

15. Mead, P. S. Epidemiology of Lyme disease. Infect. Dis. Clin. N. Amer. 2015, 29, 187-210.

16. Caulfield, A. J.; Pritt, B. S. Lyme Disease Coinfections in the United States. Clin. Lab. Med. 2015, 35, 827-846.

17. Dick, G. W.; Kitchen, S. F.; Haddow, A. J. Zika virus. I. Isolations and serological specificity. Trans. Roy. Soc. Trop. Med. Hyg. 1952, 46, 509-520.

18. Duffy, M. R.; Chen, T. H.; Hancock, W. T.; Powers, A. M.; Kool, J. L.; Lanciotti, R. S.; Pretrick, M.; Marfel, M.; Holzbauer, S.; Dubray, C.; Guillaumot, L.; Griggs, A.; Bel, M.; Lambert, A. J.; Laven, J.; Kosoy, O.; Panella, A.; Biggerstaff, B. J.; Fischer, M.; Hayes, E. B. Zika virus outbreak on Yap Island, Federated States of Micronesia. New Engl. J. Med. 2009, 360, 2536-2543.

19. Zanluca, C.; Melo, V. C.; Mosimann, A. L.; Santos, G. I.; Santos, C. N.; Luz, K. First report of autochthonous transmission of Zika virus in Brazil. Memorias do Instituto Oswaldo Cruz 2015, 110, 569-572.

20. Campos, G. S.; Bandeira, A. C.; Sardi, S. I. Zika Virus Outbreak, Bahia, Brazil. Emerg. Infect. Dis. 2015, 21, 1885-1886.

21. Hennessey, M.; Fischer, M.; Staples, J. E. Zika Virus Spreads to New Areas—Region of the Americas, May 2015-January 2016. MMWR. 2016, 65, 55-58.

22. Russo, F. B.; Jungmann, P.; Beltrao-Braga, P. C. Zika infection and the development of neurological defects. Cell. Microbiol. 2017.

23. Reynolds, M. R.; Jones, A. M.; Petersen, E. E.; Lee, E. H.; Rice, M. E.; Bingham, A.; Ellington, S. R.; Evert, N.; Reagan-Steiner, S.; Oduyebo, T.; Brown, C. M.; Martin, S.; Ahmad, N.; Bhatnagar, J.; Macdonald, J.; Gould, C.; Fine, A. D.; Polen, K. D.; Lake-Burger, H.; Hillard, C. L.; Hall, N.; Yazdy, M. M.; Slaughter, K.; Sommer, J. N.; Adamski, A.; Raycraft, M.; Fleck-Derderian, S.; Gupta, J.; Newsome, K.; Baez-Santiago, M.; Slavinski, S.; White, J. L.; Moore, C. A.; Shapiro-Mendoza, C. K.; Petersen, L.; Boyle, C.; Jamieson, D. J.; Meaney-Delman, D.; Honein, M. A. Vital Signs: Update on Zika Virus-Associated Birth Defects and Evaluation of All U.S. Infants with Congenital Zika Virus Exposure—U.S. Zika Pregnancy Registry, 2016. MMWR. 2017, 66, 366-373.

24. Cunha, A. J.; de Magalhaes-Barbosa, M. C.; Lima-Setta, F.; Medronho, R. A.; Prata-Barbosa, A. Microcephaly Case Fatality Rate Associated with Zika Virus Infection in Brazil: Current Estimates. Ped. Infect. Dis. 2017, 36, 528-530.

25. Muller, W. J.; Miller, E. S. Preliminary Results From the US Zika Pregnancy Registry: Untangling Risks for Congenital Anomalies. JAMA 2017, 317, 35-36.

26. Honein, M. A.; Dawson, A. L.; Petersen, E. E.; Jones, A. M.; Lee, E. H.; Yazdy, M. M.; Ahmad, N.; Macdonald, J.; Evert, N.; Bingham, A.; Ellington, S. R.; Shapiro-Mendoza, C. K.; Oduyebo, T.; Fine, A. D.; Brown, C. M.; Sommer, J. N.; Gupta, J.; Cavicchia, P.; Slavinski, S.; White, J. L.; Owen, S. M.; Petersen, L. R.; Boyle, C.; Meaney-Delman, D.; Jamieson, D. J. Birth Defects Among Fetuses and Infants of US Women With Evidence of Possible Zika Virus Infection During Pregnancy. JAMA 2017, 317, 59-68.

27. Cuevas, E. L.; Tong, V. T.; Rozo, N.; Valencia, D.; Pacheco, O.; Gilboa, S. M.; Mercado, M.; Renquist, C. M.; Gonzalez, M.; Ailes, E. C.; Duarte, C.; Godoshian, V.; Sancken, C. L.; Turca, A. M.; Calles, D. L.; Ayala, M.; Morgan, P.; Perez, E. N.; Bonilla, H. Q.; Gomez, R. C.; Estupinan, A. C.; Gunturiz, M. L.; Meaney-Delman, D.; Jamieson, D. J.; Honein, M. A.; Martinez, M. L. Preliminary Report of Microcephaly Potentially Associated with Zika Virus Infection During Pregnancy—Colombia, January-November 2016. MMWR. Morbidity and mortality weekly report 2016, 65, 1409-1413.

28. Collucci, C. Colombia sees fourfold increase in microcephaly cases in a year. Brit. Med. J. 2016, 355, i6716.

29. Schwartzmann, P. V.; Ramalho, L. N.; Neder, L.; Vilar, F. C.; Ayub-Ferreira, S. M.; Romeiro, M. F.; Takayanagui, O. M.; Dos Santos, A. C.; Schmidt, A.; Figueiredo, L. T.; Arena, R.; Simoes, M. V. Zika Virus Meningoencephalitis in an Immunocompromised Patient. Mayo Clin. Proc. 2017, 92, 460-466.

30. Duca, L. M.; Beckham, J. D.; Tyler, K. L.; Pastula, D. M. Zika Virus Disease and Associated Neurologic Complications. Curr. Infect. Dis. Rep. 2017, 19, 4.

31. Doughty, C. T.; Yawetz, S.; Lyons, J. Emerging Causes of Arbovirus Encephalitis in North America: Powassan, Chikungunya, and Zika Viruses. Curr. Neurol.Neurosci. Rep. 2017, 17, 12.

32. Bhatt, S.; Gething, P. W.; Brady, O. J.; Messina, J. P.; Farlow, A. W.; Moyes, C. L.; Drake, J. M.; Brownstein, J. S.; Hoen, A. G.; Sankoh, O.; Myers, M. F.; George, D. B.; Jaenisch, T.; Wint, G. R.; Simmons, C. P.; Scott, T. W.; Farrar, J. J.; Hay, S. I. The global distribution and burden of dengue. Nature 2013, 496, 504-507.

33. WHO Website (URL deleted). Global burden of dengue.

34. Succo, T.; Leparc-Goffart, I.; Ferre, J. B.; Roiz, D.; Broche, B.; Maquart, M.; Noel, H.; Catelinois, O.; Entezam, F.; Caire, D.; Jourdain, F.; Esteve-Moussion, I.; Cochet, A.; Paupy, C.; Rousseau, C.; Paty, M. C.; Golliot, F. Autochthonous dengue outbreak in Nimes, South of France, July to September 2015. Euro Surveill. 2016, 21.

35. Rogers, D. J.; Suk, J. E.; Semenza, J. C. Using global maps to predict the risk of dengue in Europe. Acta Trop. 2014, 129, 1-14.

36. Rezza, G. Dengue and other Aedes-borne viruses: a threat to Europe? Euro Surveill. 2016, 21.

37. Gupta, B.; Reddy, B. P. Fight against dengue in India: progresses and challenges. Parasitol. Res. 2013, 112, 1367-1378.

38. Chakravarti, A.; Arora, R.; Luxemburger, C. Fifty years of dengue in India. Trans. Roy. Soc. Trop. Med. Hyg. 2012, 106, 273-282.

39. Tomori, O. Yellow fever: the recurring plague. Crit. Rev. Clin. Lab. Sci. 2004, 41, 391-427.

40. Barnett, E. D. Yellow fever: epidemiology and prevention. Clin. Infect. Dis. 2007, 44, 850-856.

41. Robertson, S. E.; Hull, B. P.; Tomori, O.; Bele, O.; LeDuc, J. W.; Esteves, K. Yellow fever: a decade of reemergence. JAMA 1996, 276, 1157-1162.

42. Burke-Gaffney, H. J. Yellow fever. Trop. Dis. Bull. 1966, 63, 113-115.

43. Carey, D. E.; Kemp, G. E.; Troup, J. M.; White, H. A.; Smith, E. A.; Addy, R. F.; Fom, A. L.; Pifer, J.; Jones, E. M.; Bres, P.; Shope, R. E. Epidemiological aspects of the 1969 yellow fever epidemic in Nigeria. Bull.World Health Org. 1972, 46, 645-651.

44. Goldani, L. Z. Yellow fever outbreak in Brazil, 2017. Brazil. J. Infect. Dis. 2017, 21, 123-124.

45. Plotkin, S. A. Vaccines: the fourth century. Clin. Vaccine Immunol. 2009, 16, 1709-1719.

46. Norrby, E. Yellow fever and Max Theiler: the only Nobel Prize for a virus vaccine. J. Exp. Med. 2007, 204, 2779-2784.

47. Frierson, J. G. The yellow fever vaccine: a history. Yale J. Biol. Med. f biology and medicine 2010, 83, 77-85.

48. Cornet, M.; Robin, Y.; Hannoun, C.; Corniou, B.; Bres, P.; Causse, G. [An epidemic of yellow fever in Senegal in 1965. Epidemiological studies]. Bull.World Health Org. 1968, 39, 845-858.

49. Yellow fever, Senegal (update). Releve Epidemiol. Hebd. 2002, 77, 373-374.

50. Thomas, R. E.; Lorenzetti, D. L.; Spragins, W.; Jackson, D.; Williamson, T. Reporting rates of yellow fever vaccine 17D or 17DD-associated serious adverse events in pharmacovigilance data bases: systematic review. Curr. Drug. Safety 2011, 6, 145-154.

51. Seligman, S. J. Risk groups for yellow fever vaccine-associated viscerotropic disease (YEL-AVD). Vaccine 2014, 32, 5769-5775.

52. Munoz, J.; Vilella, A.; Domingo, C.; Nicolas, J. M.; de Ory, F.; Corachan, M.; Tenorio, A.; Gascon, J. Yellow fever-associated viscerotropic disease in Barcelona, Spain. J. Travel Med. 2008, 15, 202-205.

53. Hayes, E. B. Acute viscerotropic disease following vaccination against yellow fever. Trans. Roy. Soc. Trop. Med. Hyg. 2007, 101, 967-971.

54. Thomas, R. E.; Spragins, W.; Lorenzetti, D. L. How many published cases of serious adverse events after yellow fever vaccination meet Brighton Collaboration diagnostic criteria? Vaccine 2013, 31, 6201-6209.

55. Thomas, R. E.; Lorenzetti, D. L.; Spragins, W.; Jackson, D.; Williamson, T. Active and passive surveillance of yellow fever vaccine 17D or 17DD-associated serious adverse events: systematic review. Vaccine 2011, 29, 4544-4555.

56. Staples, J. E.; Gershman, M.; Fischer, M. Yellow fever vaccine: recommendations of the Advisory Committee on Immunization Practices (ACIP). MMWR. 2010, 59, 1-27.

57. Condit, R. C.; Williamson, A. L.; Sheets, R.; Seligman, S. J.; Monath, T. P.; Excler, J. L.; Gurwith, M.; Bok, K.; Robertson, J. S.; Kim, D.; Michael Hendry, R.; Singh, V.; Mac, L. M.; Chen, R. T. Unique safety issues associated with virus-vectored vaccines: Potential for and theoretical consequences of recombination with wild type virus strains. Vaccine 2016, 34, 6610-6616.

58. Seligman, S. J.; Gould, E. A. Live flavivirus vaccines: reasons for caution. Lancet 2004, 363, 2073-2075.

59. Francis, T., Jr. Influenza: the new acquayantance. Annals of internal medicine 1953, 39, 203-221.

60. Fazekas de St, G.; Webster, R. G. Disquisitions of Original Antigenic Sin. I. Evidence in man. The J. Exp. Med. 1966, 124, 331-345.

61. Jessie, K.; Fong, M. Y.; Devi, S.; Lam, S. K.; Wong, K. T. Localization of dengue virus in naturally infected human tissues, by immunohistochemistry and in situ hybridization. J. Infect. Dis. 2004, 189, 1411-1418.

62. Durbin, A. P.; Vargas, M. J.; Wanionek, K.; Hammond, S. N.; Gordon, A.; Rocha, C.; Balmaseda, A.; Harris, E. Phenotyping of peripheral blood mononuclear cells during acute dengue illness demonstrates infection and increased activation of monocytes in severe cases compared to classic dengue fever. Virol. 2008, 376, 429-435.

63. Zompi, S.; Harris, E. Original antigenic sin in dengue revisited. Proc. Natl. Acad. Sci. USA. 2013, 110, 8761-8762.

64. Rothman, A. L. Immunity to dengue virus: a tale of original antigenic sin and tropical cytokine storms. Nat. Rev. Immunol. 2011, 11, 532-543.

65. Kuno, G.; Gubler, D. J.; Oliver, A. Use of ‘original antigenic sin’ theory to determine the serotypes of previous dengue infections. Trans. Roy. Soc. Trop. Med. Hyg. 1993, 87, 103-105.

66. Halstead, S. B.; Rojanasuphot, S.; Sangkawibha, N. Original antigenic sin in dengue. Amer. J. Trop. Med. Hyg. 1983, 32, 154-156.

67. Morens, D. M.; Halstead, S. B. Measurement of antibody-dependent infection enhancement of four dengue virus serotypes by monoclonal and polyclonal antibodies. J. Gen. Virol. 1990, 71 ( Pt 12), 2909-2914.

68. Moi, M. L.; Takasaki, T.; Kurane, I. Human antibody response to dengue virus: implications for dengue vaccine design. Trop. Med. Health 2016, 44, 1.

69. Halstead, S. B.; O'Rourke, E. J. Dengue viruses and mononuclear phagocytes. I. Infection enhancement by non-neutralizing antibody. The J. Exp. Med. 1977, 146, 201-217.

70. Gollins, S. W.; Porterfield, J. S. Flavivirus infection enhancement in macrophages: radioactive and biological studies on the effect of antibody on viral fate. J. Gen. Virol. 1984, 65 (Pt 8), 1261-1272.

71. Balsitis, S. J.; Williams, K. L.; Lachica, R.; Flores, D.; Kyle, J. L.; Mehlhop, E.; Johnson, S.; Diamond, M. S.; Beatty, P. R.; Harris, E. Lethal antibody enhancement of dengue disease in mice is prevented by Fc modification. PLoS Path. 2010, 6, e1000790.

72. Ayala-Nunez, N. V.; Hoornweg, T. E.; van de Pol, D. P.; Sjollema, K. A.; Flipse, J.; van der Schaar, H. M.; Smit, J. M. How antibodies alter the cell entry pathway of dengue virus particles in macrophages. Sci. Rep. 2016, 6, 28768.

73. Gan, E. S.; Ting, D. H.; Chan, K. R. The mechanistic role of antibodies to dengue virus in protection and disease pathogenesis. Expert Rev. Anti-infect. Ther. 2017, 15, 111-119.

74. Hadinegoro, S. R.; Arredondo-Garcia, J. L.; Capeding, M. R.; Deseda, C.; Chotpitayasunondh, T.; Dietze, R.; Muhammad Ismail, H. I.; Reynales, H.; Limkittikul, K.; Rivera-Medina, D. M.; Tran, H. N.; Bouckenooghe, A.; Chansinghakul, D.; Cortes, M.; Fanouillere, K.; Forrat, R.; Frago, C.; Gailhardou, S.; Jackson, N.; Noriega, F.; Plennevaux, E.; Wartel, T. A.; Zambrano, B.; Saville, M. Efficacy and Long-Term Safety of a Dengue Vaccine in Regions of Endemic Disease. New Engl. J. Med. 2015, 373, 1195-1206.

75. Guy, B.; Barrere, B.; Malinowski, C.; Saville, M.; Teyssou, R.; Lang, J. From research to phase III: preclinical, industrial and clinical development of the Sanofi Pasteur tetravalent dengue vaccine. Vaccine 2011, 29, 7229-7241.

76. Simmons, C. P. A Candidate Dengue Vaccine Walks a Tightrope. New Engl. J. Med. 2015, 373, 1263-1264.

77. Paules, C. I.; Fauci, A. S. Yellow Fever—Once Again on the Radar Screen in the Americas. New Engl. J. Med. 2017, 376, 1397-1399.

78. Paul, L. M.; Carlin, E. R.; Jenkins, M. M.; Tan, A. L.; Barcellona, C. M.; Nicholson, C. O.; Michael, S. F.; Isern, S. Dengue virus antibodies enhance Zika virus infection. Clin. Transl. Immunol. 2016, 5, e117.

79. Dejnirattisai, W.; Supasa, P.; Wongwiwat, W.; Rouvinski, A.; Barba-Spaeth, G.; Duangchinda, T.; Sakuntabhai, A.; Cao-Lormeau, V. M.; Malasit, P.; Rey, F. A.; Mongkolsapaya, J.; Screaton, G. R. Dengue virus sero-cross-reactivity drives antibody-dependent enhancement of infection with zika virus. Nat. Immunol. 2016, 17, 1102-1108.

80. Castanha, P. M. S.; Nascimento, E. J. M.; Braga, C.; Cordeiro, M. T.; de Carvalho, O. V.; de Mendonca, L. R.; Azevedo, E. A. N.; Franca, R. F. O.; Dhalia, R.; Marques, E. T. A. Dengue Virus-Specific Antibodies Enhance Brazilian Zika Virus Infection. J. Infect. Dis. 2017, 215, 781-785.

81. Bardina, S. V.; Bunduc, P.; Tripathi, S.; Duehr, J.; Frere, J. J.; Brown, J. A.; Nachbagauer, R.; Foster, G. A.; Krysztof, D.; Tortorella, D.; Stramer, S. L.; Garcia-Sastre, A.; Krammer, F.; Lim, J. K. Enhancement of Zika virus pathogenesis by preexisting antiflavivirus immunity. Science (New York, N.Y.) 2017, 356, 175-180.

82. Kawiecki, A. B.; Christofferson, R. C. Zika Virus-Induced Antibody Response Enhances Dengue Virus Serotype 2 Replication In Vitro. J. Infect. Dis. 2016, 214, 1357-1360.

83. Shan, C.; Muruato, A. E.; Nunes, B. T. D.; Luo, H.; Xie, X.; Medeiros, D. B. A.; Wakamiya, M.; Tesh, R. B.; Barrett, A. D.; Wang, T.; Weaver, S. C.; Vasconcelos, P. F. C.; Rossi, S. L.; Shi, P. Y. A live-attenuated Zika virus vaccine candidate induces sterilizing immunity in mouse models. Nat. Med. 2017.

84. Larocca, R. A.; Abbink, P.; Peron, J. P.; Zanotto, P. M.; Iampietro, M. J.; Badamchi-Zadeh, A.; Boyd, M.; Ng'ang'a, D.; Kirilova, M.; Nityanandam, R.; Mercado, N. B.; Li, Z.; Moseley, E. T.; Bricault, C. A.; Borducchi, E. N.; Giglio, P. B.; Jetton, D.; Neubauer, G.; Nkolola, J. P.; Maxfield, L. F.; De La Barrera, R. A.; Jarman, R. G.; Eckels, K. H.; Michael, N. L.; Thomas, S. J.; Barouch, D. H. Vaccine protection against Zika virus from Brazil. Nature 2016, 536, 474-478.

85. Abbink, P.; Larocca, R. A.; De La Barrera, R. A.; Bricault, C. A.; Moseley, E. T.; Boyd, M.; Kirilova, M.; Li, Z.; Ng'ang'a, D.; Nanayakkara, O.; Nityanandam, R.; Mercado, N. B.; Borducchi, E. N.; Agarwal, A.; Brinkman, A. L.; Cabral, C.; Chandrashekar, A.; Giglio, P. B.; Jetton, D.; Jimenez, J.; Lee, B. C.; Mojta, S.; Molloy, K.; Shetty, M.; Neubauer, G. H.; Stephenson, K. E.; Peron, J. P.; Zanotto, P. M.; Misamore, J.; Finneyfrock, B.; Lewis, M. G.; Alter, G.; Modjarrad, K.; Jarman, R. G.; Eckels, K. H.; Michael, N. L.; Thomas, S. J.; Barouch, D. H. Protective efficacy of multiple vaccine platforms against Zika virus challenge in rhesus monkeys. Science (New York, N.Y.) 2016, 353, 1129-1132.

86. Srikiatkhachorn, A.; Yoon, I. K. Immune correlates for dengue vaccine development. Exp. Review Vaccines 2016, 15, 455-465.

87. Katzelnick, L. C.; Montoya, M.; Gresh, L.; Balmaseda, A.; Harris, E. Neutralizing antibody titers against dengue virus correlate with protection from symptomatic infection in a longitudinal cohort. Proc. Natl. Acad. Sci. USA. 2016, 113, 728-733.

88. Volz, A.; Lim, S.; Kaserer, M.; Lulf, A.; Marr, L.; Jany, S.; Deeg, C. A.; Pijlman, G. P.; Koraka, P.; Osterhaus, A. D.; Martina, B. E.; Sutter, G. Immunogenicity and protective efficacy of recombinant Modified Vaccinia virus Ankara candidate vaccines delivering West Nile virus envelope antigens. Vaccine 2016, 34, 1915-1926.

89. Tsioris, K.; Gupta, N. T.; Ogunniyi, A. O.; Zimnisky, R. M.; Qian, F.; Yao, Y.; Wang, X.; Stern, J. N.; Chari, R.; Briggs, A. W.; Clouser, C. R.; Vigneault, F.; Church, G. M.; Garcia, M. N.; Murray, K. O.; Montgomery, R. R.; Kleinstein, S. H.; Love, J. C. Neutralizing antibodies against West Nile virus identified directly from human B cells by single-cell analysis and next generation sequencing. Integr. Biol. 2015, 7, 1587-1597.

90. Di Gennaro, A.; Lorusso, A.; Casaccia, C.; Conte, A.; Monaco, F.; Savini, G. Serum neutralization assay can efficiently replace plaque reduction neutralization test for detection and quantitation of West Nile virus antibodies in human and animal serum samples. Clin. Vacc. Immunol. 2014, 21, 1460-1462.

91. Wieten, R. W.; Jonker, E. F.; van Leeuwen, E. M.; Remmerswaal, E. B.; Ten Berge, I. J.; de Visser, A. W.; van Genderen, P. J.; Goorhuis, A.; Visser, L. G.; Grobusch, M. P.; de Bree, G. J. A Single 17D Yellow Fever Vaccination Provides Lifelong Immunity; Characterization of Yellow-Fever-Specific Neutralizing Antibody and T-Cell Responses after Vaccination. PloS One 2016, 11, e0149871.

92. Julander, J. G.; Trent, D. W.; Monath, T. P. Immune correlates of protection against yellow fever determined by passive immunization and challenge in the hamster model. Vaccine 2011, 29, 6008-6016.

93. Wieten, R. W.; Goorhuis, A.; Jonker, E. F.; de Bree, G. J.; de Visser, A. W.; van Genderen, P. J.; Remmerswaal, E. B.; Ten Berge, I. J.; Visser, L. G.; Grobusch, M. P.; van Leeuwen, E. M. 17D yellow fever vaccine elicits comparable long-term immune responses in healthy individuals and immune-compromised patients. J. Infection 2016, 72, 713-722.

94. Robert Putnak, J.; Coller, B. A.; Voss, G.; Vaughn, D. W.; Clements, D.; Peters, I.; Bignami, G.; Houng, H. S.; Chen, R. C.; Barvir, D. A.; Seriwatana, J.; Cayphas, S.; Garcon, N.; Gheysen, D.; Kanesa-Thasan, N.; McDonell, M.; Humphreys, T.; Eckels, K. H.; Prieels, J. P.; Innis, B. L. An evaluation of dengue type-2 inactivated, recombinant subunit, and live-attenuated vaccine candidates in the rhesus macaque model. Vaccine 2005, 23, 4442-4452.

95. Clements, D. E.; Coller, B. A.; Lieberman, M. M.; Ogata, S.; Wang, G.; Harada, K. E.; Putnak, J. R.; Ivy, J. M.; McDonell, M.; Bignami, G. S.; Peters, I. D.; Leung, J.; Weeks-Levy, C.; Nakano, E. T.; Humphreys, T. Development of a recombinant tetravalent dengue virus vaccine: immunogenicity and efficacy studies in mice and monkeys. Vaccine 2010, 28, 2705-2715.

96. Manoff, S. B.; George, S. L.; Bett, A. J.; Yelmene, M. L.; Dhanasekaran, G.; Eggemeyer, L.; Sausser, M. L.; Dubey, S. A.; Casimiro, D. R.; Clements, D. E.; Martyak, T.; Pai, V.; Parks, D. E.; Coller, B. A. Preclinical and clinical development of a dengue recombinant subunit vaccine. Vaccine 2015, 33, 7126-7134.

97. Govindarajan, D.; Meschino, S.; Guan, L.; Clements, D. E.; ter Meulen, J. H.; Casimiro, D. R.; Coller, B. A.; Bett, A. J. Preclinical development of a dengue tetravalent recombinant subunit vaccine: Immunogenicity and protective efficacy in nonhuman primates. Vaccine 2015, 33, 4105-4116.

98. Modis, Y.; Ogata, S.; Clements, D.; Harrison, S. C. Structure of the dengue virus envelope protein after membrane fusion. Nature 2004, 427, 313-319.

99. Nayak, V.; Dessau, M.; Kucera, K.; Anthony, K.; Ledizet, M.; Modis, Y. Crystal structure of dengue virus type 1 envelope protein in the postfusion conformation and its implications for membrane fusion. J. Virol. 2009, 83, 4338-4344.

100. Zhao, H.; Fernandez, E.; Dowd, K. A.; Speer, S. D.; Platt, D. J.; Gorman, M. J.; Govero, J.; Nelson, C. A.; Pierson, T. C.; Diamond, M. S.; Fremont, D. H. Structural Basis of Zika Virus-Specific Antibody Protection. Cell 2016, 166, 1016-1027.

101. Peng, J.; Xu, J. RaptorX: exploiting structure information for protein alignment by statistical inference. Proteins 2011, 79 Suppl 10, 161-171.

102. Kallberg, M.; Wang, H.; Wang, S.; Peng, J.; Wang, Z.; Lu, H.; Xu, J. Template-based protein structure modeling using the RaptorX web server. Nat. Protocols 2012, 7, 1511-1522.

103. Kallberg, M.; Margaryan, G.; Wang, S.; Ma, J.; Xu, J. RaptorX server: a resource for template-based protein structure modeling. Meth. Molec. Biol. 2014, 1137, 17-27.

104. Qin, H.; Hu, J.; Hua, Y.; Challa, S. V.; Cross, T. A.; Gao, F. P. Construction of a series of vectors for high throughput cloning and expression screening of membrane proteins from Mycobacterium tuberculosis. BMC Biotechnol. 2008, 8, 51.

105. Nahori, M. A.; Fournie-Amazouz, E.; Que-Gewirth, N. S.; Balloy, V.; Chignard, M.; Raetz, C. R.; Saint Girons, I.; Werts, C. Differential TLR recognition of leptospiral lipid A and lipopolysaccharide in murine and human cells. J. Immunol. 2005, 175, 6022-6031.

106. Bryant, C. E.; Ouellette, A.; Lohmann, K.; Vandenplas, M.; Moore, J. N.; Maskell, D. J.; Farnfield, B. A. The cellular Toll-like receptor 4 antagonist E5531 can act as an agonist in horse whole blood. Vet Immunol. Immunopathol. 2007, 116, 182-189.

107. Byrd-Leifer, C. A.; Block, E. F.; Takeda, K.; Akira, S.; Ding, A. The role of MyD88 and TLR4 in the LPS-mimetic activity of Taxol. Eur. J. Immunol. 2001, 31, 2448-2457.

108. Kawasaki, K.; Akashi, S.; Shimazu, R.; Yoshida, T.; Miyake, K.; Nishijima, M. Involvement of TLR4/MD-2 complex in species-specific lipopolysaccharide-mimetic signal transduction by Taxol. J. Endotoxin. Res. 2001, 7, 232-236.

109. Lien, E.; Means, T. K.; Heine, H.; Yoshimura, A.; Kusumoto, S.; Fukase, K.; Fenton, M. J.; Oikawa, M.; Qureshi, N.; Monks, B.; Finberg, R. W.; Ingalls, R. R.; Golenbock, D. T. Toll-like receptor 4 imparts ligand-specific recognition of bacterial lipopolysaccharide. J. Clin. Invest. 2000, 105, 497-504.

110. Agnihotri, G.; Crall, B. M.; Lewis, T. C.; Day, T. P.; Balakrishna, R.; Warshakoon, H. J.; Malladi, S. S.; David, S. A. Structure-activity relationships in toll-like receptor 2-agonists leading to simplified monoacyl lipopeptides. J. Med. Chem. 2011, 54, 8148-8160.

111. Salunke, D. B.; Connelly, S. W.; Shukla, N. M.; Hermanson, A. R.; Fox, L. M.; David, S. A. Design and Development of Stable, Water-Soluble, Human Toll-like Receptor 2 Specific Monoacyl Lipopeptides as Candidate Vaccine Adjuvants. J. Med. Chem. 2013.

112. Heil, F.; Hemmi, H.; Hochrein, H.; Ampenberger, F.; Kirschning, C.; Akira, S.; Lipford, G.; Wagner, H.; Bauer, S. Species-specific recognition of single-stranded RNA via toll-like receptor 7 and 8. Science (New York, N.Y.) 2004, 303, 1526-1529.

113. Gorden, K. K.; Qiu, X. X.; Binsfeld, C. C.; Vasilakos, J. P.; Alkan, S. S. Cutting edge: activation of murine TLR8 by a combination of imidazoquinoline immune response modifiers and polyT oligodeoxynucleotides. J. Immunol. 2006, 177, 6584-6587.

114. Beesu, M.; Caruso, G.; Salyer, A. C.; Khetani, K. K.; Sil, D.; Weerasinghe, M.; Tanji, H.; Ohto, U.; Shimizu, T.; David, S. A. Structure-Based Design of Human TLR8-Specific Agonists with Augmented Potency and Adjuvanticity. J. Med. Chem. 2015, 58, 7833-7849.

115. Salyer, A. C.; Caruso, G.; Khetani, K. K.; Fox, L. M.; Malladi, S. S.; David, S. A. Identification of Adjuvantic Activity of Amphotericin B in a Novel, Multiplexed, Poly-TLR/NLR High-Throughput Screen. PloS One 2016, 11, e0149848.

116. Yoo, E.; Salunke, D. B.; Sil, D.; Guo, X.; Salyer, A. C.; Hermanson, A. R.; Kumar, M.; Malladi, S. S.; Balakrishna, R.; Thompson, W. H.; Tanji, H.; Ohto, U.; Shimizu, T.; David, S. A. Determinants of activity at human Toll-like receptors 7 and 8: quantitative structure-activity relationship (QSAR) of diverse heterocyclic scaffolds. J. Med. Chem. 2014, 57, 7955-7970.

117. Kokatla, H. P.; Yoo, E.; Salunke, D. B.; Sil, D.; Ng, C. F.; Balakrishna, R.; Malladi, S. S.; Fox, L. M.; David, S. A. Toll-like receptor-8 agonistic activities in C2, C4, and C8 modified thiazolo[4,5-c]quinolines. Org. Biomol. Chem. 2013, 11, 1179-1198.

118. Salunke, D. B.; Shukla, N. M.; Yoo, E.; Crall, B. M.; Balakrishna, R.; Malladi, S. S.; David, S. A. Structure-Activity Relationships in Human Toll-like Receptor 2-Specific Monoacyl Lipopeptides. J. Med. Chem. 2012, 55, 3353-3363.

119. Salunke, D. B.; Yoo, E.; Shukla, N. M.; Balakrishna, R.; Malladi, S. S.; Serafin, K. J.; Day, V. W.; Wang, X.; David, S. A. Structure-activity relationships in human Toll-like receptor 8-active 2,3-diamino-furo[2,3-c]pyridines. J. Med. Chem. 2012, 55, 8137-8151.

120. Shukla, N. M.; Salunke, D. B.; Balakrishna, R.; Mutz, C. A.; Malladi, S. S.; David, S. A. Potent adjuvanticity of a pure TLR7-agonistic imidazoquinoline dendrimer. PloS one 2012, 7, e43612.

121. Guidance for Industry: Considerations for Developmental Toxicity Studies for Preventive and Therapeutic Vaccines for Infectious Disease Indications. (FDA website. URL deleted).

122. Ukani, R.; Lewis, T. C.; Day, T. P.; Wu, W.; Malladi, S. S.; Warshakoon, H. J.; David, S. A. Potent adjuvantic activity of a CCR1-agonistic bis-quinoline. Bioorg. Med. Chem. Lett. 2012, 22, 293-295.

123. Daniels, J. B.; Ratner, J. J.; Brown, S. R. Plaque reduction, a sensitive test for eastern encephalitis antibody. Science (New York, N.Y.) 1961, 133, 640-641.

124. Chatchen, S.; Sabchareon, A.; Sirivichayakul, C. Serodiagnosis of asymptomatic dengue infection. Asian Pac. J. Trop. Med. 2017, 10, 11-14.

125. Shan, C.; Xie, X.; Ren, P.; Loeffelholz, M. J.; Yang, Y.; Furuya, A.; Dupuis, A. P., 2nd; Kramer, L. D.; Wong, S. J.; Shi, P. Y. A Rapid Zika Diagnostic Assay to Measure Neutralizing Antibodies in Patients. EBioMedicine 2017, 17, 157-162.

126. Pascoalino, B. S.; Courtemanche, G.; Cordeiro, M. T.; Gil, L. H.; Freitas-Junior, L. Zika antiviral chemotherapy: identification of drugs and promising starting points for drug discovery from an FDA-approved library. F1000Research 2016, 5, 2523.

127. Goebel, S.; Snyder, B.; Sellati, T.; Saeed, M.; Ptak, R.; Murray, M.; Bostwick, R.; Rayner, J.; Koide, F.; Kalkeri, R. A sensitive virus yield assay for evaluation of Antivirals against Zika Virus. J. Virol. Meth. 2016, 238, 13-20.

128. Adcock, R. S.; Chu, Y. K.; Golden, J. E.; Chung, D. H. Evaluation of anti-Zika virus activities of broad-spectrum antivirals and NIH clinical collection compounds using a cell-based, high-throughput screen assay. Antivir. Res. 2017, 138, 47-56.

129. Xu, M.; Lee, E. M.; Wen, Z.; Cheng, Y.; Huang, W. K.; Qian, X.; Tcw, J.; Kouznetsova, J.; Ogden, S. C.; Hammack, C.; Jacob, F.; Nguyen, H. N.; Itkin, M.; Hanna, C.; Shinn, P.; Allen, C.; Michael, S. G.; Simeonov, A.; Huang, W.; Christian, K. M.; Goate, A.; Brennand, K. J.; Huang, R.; Xia, M.; Ming, G. L.; Zheng, W.; Song, H.; Tang, H. Identification of small-molecule inhibitors of Zika virus infection and induced neural cell death via a drug repurposing screen. Nat. Med. 2016, 22, 1101-1107.

130. Stiasny, K.; Kiermayr, S.; Holzmann, H.; Heinz, F. X. Cryptic properties of a cluster of dominant flavivirus cross-reactive antigenic sites. J. Virol. 2006, 80, 9557-9568.

131. Crill, W. D.; Chang, G. J. Localization and characterization of flavivirus envelope glycoprotein cross-reactive epitopes. J. Virol. 2004, 78, 13975-13986.

132. Megret, F.; Hugnot, J. P.; Falconar, A.; Gentry, M. K.; Morens, D. M.; Murray, J. M.; Schlesinger, J. J.; Wright, P. J.; Young, P.; Van Regenmortel, M. H.; et al. Use of recombinant fusion proteins and monoclonal antibodies to define linear and discontinuous antigenic sites on the dengue virus envelope glycoprotein. Virology 1992, 187, 480-491.

133. Sun, P.; Tropea, J. E.; Waugh, D. S. Enhancing the solubility of recombinant proteins in Escherichia coli by using hexahistidine-tagged maltose-binding protein as a fusion partner. Methods Molec. Biol. (Clifton, N.J.) 2011, 705, 259-274.

134. Lin, Z.; Zhao, Q.; Xing, L.; Zhou, B.; Wang, X. Aggregating tags for column-free protein purification. Biotechnol. J. 2015, 10, 1877-1886.

135. Lebendiker, M.; Danieli, T. Purification of Proteins Fused to Maltose-Binding Protein. Methods Molec. Biol. 2017, 1485, 257-273.

136. Kim, Y.; Babnigg, G.; Jedrzejczak, R.; Eschenfeldt, W. H.; Li, H.; Maltseva, N.; Hatzos-Skintges, C.; Gu, M.; Makowska-Grzyska, M.; Wu, R.; An, H.; Chhor, G.; Joachimiak, A. High-throughput protein purification and quality assessment for crystallization. Methods) 2011, 55, 12-28.

137. Cesaratto, F.; Burrone, O. R.; Petris, G. Tobacco Etch Virus protease: A shortcut across biotechnologies. J. Biotechnol. 2016, 231, 239-249.

138. Bodenhausen, G.; Ruben, D. J. Natural abundance nitrogen-15 NMR by enhanced heteronuclear spectroscopy. Chem. Phys. Lett. 1980, 69, 185-189.

139. Nettleship, J. E.; Brown, J.; Groves, M. R.; Geerlof, A. Methods for protein characterization by mass spectrometry, thermal shift (ThermoFluor) assay, and multiangle or static light scattering. Methods Molec. Biol. 2008, 426, 299-318.

140. Huynh, K.; Partch, C. L. Analysis of protein stability and ligand interactions by thermal shift assay. Current Protocols Prot. Sci. 2015, 79, 28.29.21-14.

141. Osuna, C. E.; Lim, S. Y.; Deleage, C.; Griffin, B. D.; Stein, D.; Schroeder, L. T.; Omange, R.; Best, K.; Luo, M.; Hraber, P. T.; Andersen-Elyard, H.; Ojeda, E. F.; Huang, S.; Vanlandingham, D. L.; Higgs, S.; Perelson, A. S.; Estes, J. D.; Safronetz, D.; Lewis, M. G.; Whitney, J. B. Zika viral dynamics and shedding in rhesus and cynomolgus macaques. Nat. Med. 2016, 22, 1448-1455.

142. Meertens, L.; Labeau, A.; Dejarnac, 0.; Cipriani, S.; Sinigaglia, L.; Bonnet-Madin, L.; Le Charpentier, T.; Hafirassou, M. L.; Zamborlini, A.; Cao-Lormeau, V. M.; Coulpier, M.; Misse, D.; Jouvenet, N.; Tabibiazar, R.; Gressens, P.; Schwartz, O.; Amara, A. Axl Mediates ZIKA Virus Entry in Human Glial Cells and Modulates Innate Immune Responses. Cell Rep. 2017, 18, 324-333.

143. Noronha, L.; Zanluca, C.; Azevedo, M. L.; Luz, K. G.; Santos, C. N. Zika virus damages the human placental barrier and presents marked fetal neurotropism. Memorias do Instituto Oswaldo Cruz 2016, 111, 287-293.

144. Moreira, J.; Peixoto, T. M.; Siqueira, A. M.; Lamas, C. C. Sexually acquired Zika virus: a systematic review. Clin. Microbiol. Infect. 2017. 23:296-305

145. Hamer, D. H.; Wilson, M. E.; Jean, J.; Chen, L. H. Epidemiology, Prevention, and Potential Future Treatments of Sexually Transmitted Zika Virus Infection. Curr. Infect. Dis. Rep. 2017, 19, 16.

146. Mossenta, M.; Marchese, S.; Poggianella, M.; Slon Campos, J. L.; Burrone, O. R. Role of N-glycosylation on Zika virus E protein secretion, viral assembly and infectivity. Biochem. Biophys. Res. Commun. 2017. doi: 10.1016/j.bbrc.2017.01.022.

147. Zai, J.; Mei, L.; Wang, C.; Cao, S.; Fu, Z. F.; Chen, H.; Song, Y. N-glycosylation of the premembrane protein of Japanese encephalitis virus is critical for folding of the envelope protein and assembly of virus-like particles. Acta Virol. 2013, 57, 27-33.

148. Roby, J. A.; Setoh, Y. X.; Hall, R. A.; Khromykh, A. A. Post-translational regulation and modifications of flavivirus structural proteins. J. Gen. Virol. 2015, 96, 1551-1569.

149. Naik, N. G.; Wu, H. N. Mutation of Putative N-Glycosylation Sites on Dengue Virus NS4B Decreases RNA Replication. J. Virol. 2015, 89, 6746-6760.

150. Mondotte, J. A.; Lozach, P. Y.; Amara, A.; Gamarnik, A. V. Essential role of dengue virus envelope protein N glycosylation at asparagine-67 during viral propagation. J. Virol. 2007, 81, 7136-7148.

151. Hanna, S. L.; Pierson, T. C.; Sanchez, M. D.; Ahmed, A. A.; Murtadha, M. M.; Doms, R. W. N-linked glycosylation of west nile virus envelope proteins influences particle assembly and infectivity. J. Virol. 2005, 79, 13262-13274.

152. Goto, A.; Yoshii, K.; Obara, M.; Ueki, T.; Mizutani, T.; Kariwa, H.; Takashima, I. Role of the N-linked glycans of the prM and E envelope proteins in tick-borne encephalitis virus particle secretion. Vaccine 2005, 23, 3043-3052.

153. Davis, C. W.; Mattei, L. M.; Nguyen, H. Y.; Ansarah-Sobrinho, C.; Doms, R. W.; Pierson, T. C. The location of asparagine-linked glycans on West Nile virions controls their interactions with CD209 (dendritic cell-specific ICAM-3 grabbing nonintegrin). J. Biol. Chem. 2006, 281, 37183-37194.

154. Barba-Spaeth, G.; Dejnirattisai, W.; Rouvinski, A.; Vaney, M. C.; Medits, I.; Sharma, A.; Simon-Loriere, E.; Sakuntabhai, A.; Cao-Lormeau, V. M.; Haouz, A.; England, P.; Stiasny, K.; Mongkolsapaya, J.; Heinz, F. X.; Screaton, G. R.; Rey, F. A. Structural basis of potent Zika-dengue virus antibody cross-neutralization. Nature 2016, 536, 48-53.

155. Ganapathi, L.; Van Haren, S.; Dowling, D. J.; Bergelson, I.; Shukla, N. M.; Malladi, S. S.; Balakrishna, R.; Tanji, H.; Ohto, U.; Shimizu, T.; David, S. A.; Levy, O. The Imidazoquinoline Toll-Like Receptor-7/8 Agonist Hybrid-2 Potently Induces Cytokine Production by Human Newborn and Adult Leukocytes. PloS One 2015, 10, e0134640.

156. Shukla, N. M.; Malladi, S. S.; Mutz, C. A.; Balakrishna, R.; David, S. A. Structure-activity relationships in human toll-like receptor 7-active imidazoquinoline analogues. J. Med. Chem. 2010, 53, 4450-4465.

157. Liao, S.; von der Weid, P. Y. Lymphatic system: An active pathway for immune protection. Semin. Cell Develop. Biol. 2015. 38:83-89.

158. Swartz, M. A.; Hubbell, J. A.; Reddy, S. T. Lymphatic drainage function and its immunological implications: from dendritic cell homing to vaccine design. Semin. Immunol. 2008, 20, 147-156.

159. Waeckerle-Men, Y.; Bruffaerts, N.; Liang, Y.; Jurion, F.; Sander, P.; Kundig, T. M.; Huygen, K.; Johansen, P. Lymph node targeting of BCG vaccines amplifies CD4 and CD8 T-cell responses and protection against Mycobacterium tuberculosis. Vaccine 2013, 31, 1057-1064.

160. Pal, I.; Ramsey, J. D. The role of the lymphatic system in vaccine trafficking and immune response. Adv. Drug. Deliv. Res. 2011, 63, 909-922.

161. Jiang, D.; Liang, J.; Noble, P. W. Hyaluronan as an immune regulator in human diseases. Physiol. Rev. 2011, 91, 221.

162. Jackson, D. G. Immunological functions of hyaluronan and its receptors in the lymphatics. Immunol. Rev. 2009, 230, 216-231.

163. Schanté, C. E.; Zuber, G.; Herlin, C.; Vandamme, T. F. Chemical modifications of hyaluronic acid for the synthesis of derivatives for a broad range of biomedical applications. Carb. Polym. 2011, 85, 469-489.

164. Mero, A.; Campisi, M. Hyaluronic acid bioconjugates for the delivery of bioactive molecules. Polymers 2014, 6, 346-369.

165. Misra, S.; Heldin, P.; Hascall, V. C.; Karamanos, N. K.; Skandalis, S. S.; Markwald, R. R.; Ghatak, S. Hyaluronan-CD44 interactions as potential targets for cancer therapy. FEBS J. 2011, 278, 1429-1443.

166. Jackson, D. G. Biology of the lymphatic marker LYVE-1 and applications in research into lymphatic trafficking and lymphangiogenesis. APMIS 2004, 112, 526-538.

167. Wang, C.; Liu, P.; Zhuang, Y.; Li, P.; Jiang, B.; Pan, H.; Liu, L.; Cai, L.; Ma, Y. Lymphatic-targeted cationic liposomes: a robust vaccine adjuvant for promoting long-term immunological memory. Vaccine 2014, 32, 5475-5483.

168. Liu, H.; Moynihan, K. D.; Zheng, Y.; Szeto, G. L.; Li, A. V.; Huang, B.; Van Egeren, D. S.; Park, C.; Irvine, D. J. Structure-based programming of lymph-node targeting in molecular vaccines. Nature 2014, 507, 519-522.

169. Gupta, R. K. Aluminum compounds as vaccine adjuvants. Adv. Drug. Deliv. Rev. 1998, 32, 155-172.

170. Bachmann, M. F.; Jennings, G. T. Vaccine delivery: a matter of size, geometry, kinetics and molecular patterns. Nat. Rev. Immunol. 2010, 10, 787-796.

171. De Temmerman, M. L.; Rejman, J.; Demeester, J.; Irvine, D. J.; Gander, B.; De Smedt, S. C. Particulate vaccines: on the quest for optimal delivery and immune response. Drug Disc. Today 2011, 16, 569-582.

172. Reddy, S. T.; van der Vlies, A. J.; Simeoni, E.; Angeli, V.; Randolph, G. J.; O'Neil, C. P.; Lee, L. K.; Swartz, M. A.; Hubbell, J. A. Exploiting lymphatic transport and complement activation in nanoparticle vaccines. Nat. Biotechnol. 2007, 25, 1159-1164.

173. Oh, E. J.; Park, K.; Kim, K. S.; Kim, J.; Yang, J. A.; Kong, J. H.; Lee, M. Y.; Hoffman, A. S.; Hahn, S. K. Target specific and long-acting delivery of protein, peptide, and nucleotide therapeutics using hyaluronic acid derivatives. J. Control. Release 2010, 141, 2-12.

174. Yoo, E.; Crall, B. M.; Balakrishna, R.; Malladi, S. S.; Fox, L. M.; Hermanson, A. R.; David, S. A. Structure-activity relationships in Toll-like receptor 7 agonistic 1H-imidazo[4,5-c]pyridines. Org. Biomol. Chem. 2013, 11, 6526-6545.

175. Shukla, N. M.; Kimbrell, M. R.; Malladi, S. S.; David, S. A. Regioisomerism-dependent TLR7 agonism and antagonism in an imidazoquinoline. Bioorg. Med. Chem. Lett. 2009, 19, 2211-2214.

176. Beesu, M.; Salyer, A. C.; Trautman, K. L.; Hill, J. K.; David, S. A. Human Toll-like Receptor (TLR) 8-Specific Agonistic Activity in Substituted Pyrimidine-2,4-diamines. J. Med. Chem. 2016, 59, 8082-8093.

177. Beesu, M.; Malladi, S. S.; Fox, L. M.; Jones, C. D.; Dixit, A.; David, S. A. Human Toll-like receptor 8-selective agonistic activities in 1-alkyl-1H-benzimidazol-2-amines. J. Med. Chem. 2014, 57, 7325-7341.

178. Shukla, N. M.; Mutz, C. A.; Ukani, R.; Warshakoon, H. J.; Moore, D. S.; David, S. A. Syntheses of fluorescent imidazoquinoline conjugates as probes of Toll-like receptor 7. Bioorg. Med. Chem. Lett. 2010, 20, 6384-6386.

179. Shukla, N. M.; Lewis, T. C.; Day, T. P.; Mutz, C. A.; Ukani, R.; Hamilton, C. D.; Balakrishna, R.; David, S. A. Toward self-adjuvanting subunit vaccines: model peptide and protein antigens incorporating covalently bound toll-like receptor-7 agonistic imidazoquinolines. Bioorg. Med. Chem. Lett.2011, 21, 3232-3236.

180. Bergman, K.; Elvingson, C.; Hilborn, J.; Svensk, G.; Bowden, T. Hyaluronic acid derivatives prepared in aqueous media by triazine-activated amidation. Biomacromol. 2007, 8, 2190-2195.

181. Borke, T.; Winnik, F. M.; Tenhu, H.; Hietala, S. Optimized triazine-mediated amidation for efficient and controlled functionalization of hyaluronic acid. Carbohydr. Polym. 2015, 116, 42-50.

182. Hood, J. D.; Warshakoon, H. J.; Kimbrell, M. R.; Shukla, N. M.; Malladi, S. S.; Wang, X.; David, S. A. Immunoprofiling toll-like receptor ligands: Comparison of immunostimulatory and proinflammatory profiles in ex vivo human blood models. Hum. Vacc. 2010, 6, 322-335.

183. Warshakoon, H. J.; Hood, J. D.; Kimbrell, M. R.; Malladi, S.; Wu, W. Y.; Shukla, N. M.; Agnihotri, G.; Sil, D.; David, S. A. Potential adjuvantic properties of innate immune stimuli. Hum. Vacc. 2009, 5, 381-394.

184. Malito, E.; Bursulaya, B.; Chen, C.; Lo, S. P.; Picchianti, M.; Balducci, E.; Biancucci, M.; Brock, A.; Berti, F.; Bottomley, M. J.; Nissum, M.; Costantino, P.; Rappuoli, R.; Spraggon, G. Structural basis for lack of toxicity of the diphtheria toxin mutant CRM197. Proc.Natl.Acad.Sci.U.S.A 2012, 109, 5229-5234.

185. Pappenheimer, A. M. Studies on Diphtheria Toxin and Its Reaction with Antitoxin. J. Bacteriol. 1942, 43, 273-289.

186. Pappenheimer, A. M., Jr. The story of a toxic protein, 1888-1992. Prot. Sci. 1993, 2, 292-298.

187. Bachmann, M. F.; Kalinke, U.; Althage, A.; Freer, G.; Burkhart, C.; Roost, H.; Aguet, M.; Hengartner, H.; Zinkernagel, R. M. The role of antibody concentration and avidity in antiviral protection. Science 1997, 276, 2024-2027.

188. Pullen, G. R.; Fitzgerald, M. G.; Hosking, C. S. Antibody avidity determination by ELISA using thiocyanate elution. J. Immunol. Meth. 1986, 86, 83-87.

189. Harris, S. L.; Tsao, H.; Ashton, L.; Goldblatt, D.; Fernsten, P. Avidity of the immunoglobulin G response to a Neisseria meningitidis group C polysaccharide conjugate vaccine as measured by inhibition and chaotropic enzyme-linked immunosorbent assays. Clin. Vacc. Immunol.: CVI 2007, 14, 397-403.

190. Nossal, G. J. Kinetics of antibody formation and regulatory aspects of immunity. Acta Endocrin. Suppl. 1975, 194, 96-116.

191. Berek, C.; Milstein, C. Mutation drift and repertoire shift in the maturation of the immune response. Immunol. Rev. 1987, 96, 23-41.

192. Pockros, P. J.; Guyader, D.; Patton, H.; Tong, M. J.; Wright, T.; McHutchison, J. G.; Meng, T. C. Oral resiquimod in chronic HCV infection: safety and efficacy in 2 placebo-controlled, double-blind phase IIa studies. J. Hepatol. 2007, 47, 174-182.

193. Sauder, D. N.; Smith, M. H.; Senta-McMillian, T.; Soria, I.; Meng, T. C. Randomized, single-blind, placebo-controlled study of topical application of the immune response modulator resiquimod in healthy adults. Antimicrob. Agents Chemother. 2003, 47, 3846-3852.

194. Szeimies, R. M.; Bichel, J.; Ortonne, J. P.; Stockfleth, E.; Lee, J.; Meng, T. C. A phase II dose-ranging study of topical resiquimod to treat actinic keratosis. Brit. J. Dermatol. 2008, 159, 205-210.

195. Wu, T. Y.; Singh, M.; Miller, A. T.; De Gregorio, E.; Doro, F.; D'Oro, U.; Skibinski, D. A.; Mbow, M. L.; Bufali, S.; Herman, A. E.; Cortez, A.; Li, Y.; Nayak, B. P.; Tritto, E.; Filippi, C. M.; Otten, G. R.; Brito, L. A.; Monaci, E.; Li, C.; Aprea, S.; Valentini, S.; Calabromicron, S.; Laera, D.; Brunelli, B.; Caproni, E.; Malyala, P.; Panchal, R. G.; Warren, T. K.; Bavari, S.; O'Hagan, D. T.; Cooke, M. P.; Valiante, N. M. Rational design of small molecules as vaccine adjuvants. Sci. Transl. Med. 2014, 6, 263ra160.

196. Petrovsky, N.; Aguilar, J. C. Vaccine adjuvants: current state and future trends. Immunol. Cell Biol. 2004, 82, 488-496.

197. Lambrecht, B. N.; Kool, M.; Willart, M. A.; Hammad, H. Mechanism of action of clinically approved adjuvants. Curr. Opin. Immunol. 2009, 21, 23-29.

198. Kuroda, E.; Coban, C.; Ishii, K. J. Particulate adjuvant and innate immunity: past achievements, present findings, and future prospects. Int. Rev. Immunol.2013, 32, 209-220.

199. He, P.; Zou, Y.; Hu, Z. Advances in aluminum hydroxide-based adjuvant research and its mechanism. Hum. Vacc. Immunother. 2015, 11, 477-488.

200. Bishop, N. J.; Morley, R.; Day, J. P.; Lucas, A. Aluminum neurotoxicity in preterm infants receiving intravenous-feeding solutions. New Engl. J. Med. 1997, 336, 1557-1561.

201. Fanni, D.; Ambu, R.; Gerosa, C.; Nemolato, S.; lacovidou, N.; Van Eyken, P.; Fanos, V.; Zaffanello, M.; Faa, G. Aluminum exposure and toxicity in neonates: a practical guide to halt aluminum overload in the prenatal and perinatal periods. World J. Pediatr.: WJP 2014, 10, 101-107.

202. Bohrer, D.; Oliveira, S. M.; Garcia, S. C.; Nascimento, P. C.; Carvalho, L. M. Aluminum loading in preterm neonates revisited. J. Pediatr. Gastroent. Nutr. 2010, 51, 237-241.

203. Nuhn, L.; Vanparijs, N.; De Beuckelaer, A.; Lybaert, L.; Verstraete, G.; Deswarte, K.; Lienenklaus, S.; Shukla, N. M.; Salyer, A. C.; Lambrecht, B. N.; Grooten, J.; David, S. A.; De Koker, S.; De Geest, B. G. pH-degradable imidazoquinoline-ligated nanogels for lymph node-focused immune activation. Proc. Natl. Acad. Sci. USA. 2016, 113, 8098-8103.

204. Lammermann, T.; Sixt, M. The microanatomy of T-cell responses. lmmunol. Rev. 2008, 221, 26-43.

205. Rantakari, P.; Auvinen, K.; Jappinen, N.; Kapraali, M.; Valtonen, J.; Karikoski, M.; Gerke, H.; Iftakhar, E. K. I.; Keuschnigg, J.; Umemoto, E.; Tohya, K.; Miyasaka, M.; Elima, K.; Jalkanen, S.; Salmi, M. The endothelial protein PLVAP in lymphatics controls the entry of lymphocytes and antigens into lymph nodes. Nat. Immunol. 2015, 16, 386-396.

206. Hons, M.; Sixt, M. The lymph node filter revealed. Nat Immunol 2015, 16, 338-340.

207. Junt, T.; Moseman, E. A.; Iannacone, M.; Massberg, S.; Lang, P. A.; Boes, M.; Fink, K.; Henrickson, S. E.; Shayakhmetov, D. M.; Di Paolo, N. C.; van Rooijen, N.; Mempel, T. R.; Whelan, S. P.; von Andrian, U. H. Subcapsular sinus macrophages in lymph nodes clear lymph-borne viruses and present them to antiviral B cells. Nature 2007, 450, 110-114.

208. Phan, T. G.; Grigorova, I.; Okada, T.; Cyster, J. G. Subcapsular encounter and complement-dependent transport of immune complexes by lymph node B cells. Nat. Immunol. 2007, 8, 992-1000.

209. Cantor, J. O.; Cerreta, J. M.; Armand, G.; Osman, M.; Turino, G. M. The pulmonary matrix, glycosaminoglycans and pulmonary emphysema. Connect. Tissue Res. 1999, 40, 97-104.

210. Lienenklaus, S.; Cornitescu, M.; Zietara, N.; Lyszkiewicz, M.; Gekara, N.; Jablonska, J.; Edenhofer, F.; Rajewsky, K.; Bruder, D.; Hafner, M.; Staeheli, P.; Weiss, S. Novel reporter mouse reveals constitutive and inflammatory expression of IFN-beta in vivo. J. Immunol. 2009, 183, 3229-3236.

211. Kantner, T.; Watts, A. G. Characterization of Reactions between Water-Soluble Trialkylphosphines and Thiol Alkylating Reagents: Implications for Protein-Conjugation Reactions. Bioconj. Chem. 2016, 27, 2400-2406.

212. Michaelis, L. Weitere Untersuchungen uber Eiweissprazipitine. Deutsch. Med. Wochenschr. 1904, 30, 1240.

213. Michaelis, L. Untersuchungen uber Eiweissprazipitine. Deutsch. Med. Wochenschr. 1902, 28, 733.

214. Weigle, W. O.; High, G. J. The effect of antigenic competition on antibody production to heterologous proteins, termination of immunologic unresponsiveness and induction of autoimmunity. J. Immunol. 1967, 99, 392-398.

215. Brody, N. I.; Siskind, G. W. Studies on antigenic competition. J. Exp. Med. 1969, 130, 821-832.

216. Schechter, I. Competition of antigenic determinants. Biochim. Biophys. Acta 1965, 104, 303-305.

217. Adler, F. L. Antibody formation after injection of heterologous immune globulin. II. Competition of antigens. J. Immunol. 1957, 78, 201-210.

218. McGuire, A. T.; Dreyer, A. M.; Carbonetti, S.; Lippy, A.; Glenn, J.; Scheid, J. F.; Mouquet, H.; Stamatatos, L. HIV antibodies. Antigen modification regulates competition of broad and narrow neutralizing HIV antibodies. Science 2014, 346, 1380-1383.

219. Garcia, Z.; Pradelli, E.; Celli, S.; Beuneu, H.; Simon, A.; Bousso, P. Competition for antigen determines the stability of T cell-dendritic cell interactions during clonal expansion. Proc. Natl. Acad. Sci. USA. 2007, 104, 4553-4558.

220. Farrington, L. A.; Smith, T. A.; Grey, F.; Hill, A. B.; Snyder, C. M. Competition for antigen at the level of the APC is a major determinant of immunodominance during memory inflation in murine cytomegalovirus infection. J. Immunol. 2013, 190, 3410-3416.

221. Blair, D. A.; Lefrancois, L. Increased competition for antigen during priming negatively impacts the generation of memory CD4 T cells. Proc. Natl. Acad. Sci. USA. 2007, 104, 15045-15050.

222. Wang, P.; Shih, C. M.; Qi, H.; Lan, Y. H. A Stochastic Model of the Germinal Center Integrating Local Antigen Competition, Individualistic T-B Interactions, and B Cell Receptor Signaling. J. Immunol. 2016, 197, 1169-1182.

223. Thierry-Carstensen, B.; Dalby, T.; Stevner, M. A.; Robbins, J. B.; Schneerson, R.; Trollfors, B. Experience with monocomponent acellular pertussis combination vaccines for infants, children, adolescents and adults—a review of safety, immunogenicity, efficacy and effectiveness studies and 15 years of field experience. Vaccine 2013, 31, 5178-5191.

224. Yuen, C. T.; Asokanathan, C.; Cook, S.; Lin, N.; Xing, D. Effect of different detoxification procedures on the residual pertussis toxin activities in vaccines. Vaccine 2016, 34, 2129-2134.

225. Habeeb, A. J.; Hiramoto, R. Reaction of proteins with glutaraldehyde. Arch. Biochem. Biophys. 1968, 126, 16-26.

226. Migneault, I.; Dartiguenave, C.; Bertrand, M. J.; Waldron, K. C. Glutaraldehyde: behavior in aqueous solution, reaction with proteins, and application to enzyme crosslinking. Bio Techniques 2004, 37, 790-796, 798-802.

227. Adediran, S. A.; Day, T. P.; Sil, D.; Kimbrell, M. R.; Warshakoon, H. J.; Malladi, S. S.; David, S. A. Synthesis of a highly water-soluble derivative of amphotericin B with attenuated proinflammatory activity. Mol. Pharm. 2009, 6, 1582-1590.

228. Day, T. P.; Sil, D.; Shukla, N. M.; Anbanandam, A.; Day, V. W.; David, S. A. Imbuing aqueous solubility to amphotericin B and nystatin with a vitamin. Mol. Pharm. 2011, 8, 297-301.

229. Clapp, T.; Siebert, P.; Chen, D.; Jones Braun, L. Vaccines with aluminum-containing adjuvants: optimizing vaccine efficacy and thermal stability. J. Pharm. Sci. 2011, 100, 388-401.

230. Brandau, D. T.; Jones, L. S.; Wiethoff, C. M.; Rexroad, J.; Middaugh, C. R. Thermal stability of vaccines. J. Pharm. Sci. 2003, 92, 218-231.

231. Barrow, P. Developmental and reproductive toxicity testing of vaccines. J. Pharm. Toxicol. Meth. 2012, 65, 58-63.

232. Verdier, F.; Barrow, P. C.; Burge, J. Reproductive toxicity testing of vaccines. Toxicol. 2003, 185, 213-219.

233. Barrow, P. C.; Allais, L. Developmental toxicity testing of vaccines. Methods Molec. Biol. 2013, 947, 81-89.

234. Barrow, P. C. Reproductive toxicology studies and immunotherapeutics. Toxicol. 2003, 185, 205-212.

The complete disclosure of all patents, patent applications, and publications, and electronically available material (including, for instance, nucleotide sequence submissions in, for example, GenBank and RefSeq, and amino acid sequence submissions in, for example, SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A subunit vaccine for a flavivirus, the vaccine comprising an antigen, the antigen comprising a sequence having at least 80% sequence identity to one of SEQ ID NO:1 to SEQ ID NO:12.

2. The subunit vaccine of claim 1, wherein the antigen comprises a sequence having at least 90% sequence identity to one of SEQ ID NO:1 to SEQ ID NO:12.

3-6. (canceled)

7. The subunit vaccine of claim 1, wherein the flavivirus comprises at least one of Zika virus (ZIKV), dengue virus (DENV), Yellow Fever (YF) virus, and West Nile Virus (WNV).

8-18. (canceled)

19. A pharmaceutical composition comprising:
the subunit vaccine of claim 1, and
a pharmaceutically acceptable carrier.

20. The composition of claim 19, the composition further comprising an adjuvant.

21. The composition of claim 20, the adjuvant comprising hyaluronic acid.

22. The composition of claim 20, the adjuvant comprising a TLR agonist.

23. The composition of claim 22, the TLR agonist comprising at least one of a TLR 7 and a TLR 8 agonist.

24. The composition of claim 22, the TLR agonist comprising [5-(3-(aminomethyl)benzyl)-3-pentylquinolin-2-amine].

25. The composition of claim 20, the adjuvant comprising a covalent conjugate of hyaluronic acid and a TLR agonist.

26. The composition of claim 20, the adjuvant comprising

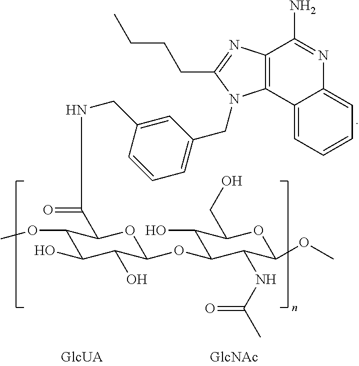

27-34. (canceled)

35. A method of making the subunit vaccine of claim 1.

36. The method of claim 35, wherein the method comprises expressing a construct comprising a sequence encoding the antigen, wherein the resulting antigen is operably linked to a tag.

37. The method of claim 35, wherein the the method comprises expressing a construct comprising the antigen and a sequence encoding a tag, wherein the construct further comprises a protease cleavage site between the sequence encoding the antigen and the sequence encoding the tag.

38. The method of claim 37, wherein the protease cleavage site comprises a TEV protease cleavage site.

39. The method of claim 36, wherein the tag comprises a maltose binding protein (MBP), a small ubiquitin-like modifier (SUMO), a Glutathione S-transferase (GST), a Streptococcal G protein (SGp), or combinations and/or portions thereof.

40. The method of claim 36, wherein the tag comprises SEQ ID NO:13 or SEQ ID NO:14.

41. (canceled)

42. The method of claim 36, the method further comprising cleaving the tag from the antigen.

43. The method of claim 42, wherein the tag is cleaved from the antigen by TEV protease.

44. A method comprising administering the subunit vaccine of claim 1.

45-60. (canceled)
